SEQUENCE LISTING (5 sequences)

SEQ ID NO: 1 - protein, 37 aa, Homo sapiens
LLGDFFRKSKEKIGKEFKRIVQRIKDFLRNLVPRTES

SEQ ID NO: 2 - DNA, 22 bp, Artificial Sequence
  RNase L forward primer
ggaggagaagctttacaaggtg

SEQ ID NO: 3 - DNA, 20 bp, Artificial Sequence
  RNase L reverse primer
gcattgaggaccatggagac

SEQ ID NO: 4 - DNA, 21 bp, Artificial Sequence
  IL36R forward primer
gccgctacacaccacaaccag

SEQ ID NO: 5 - DNA, 23 bp, Artificial Sequence
  IL36R reverse primer
agttcagtagtccactgccactc

(19) United States
(12) Patent Application Publication
(10) Pub. No.: US 2023/0218705 A1
(43) Pub. Date: Jul. 13, 2023

(54) CASPASE INHIBITORS TO ENHANCE INJURY REPAIR AND TO TREAT BACTERIAL AND VIRAL INFECTIONS

(71) Applicant: The Johns Hopkins University, Baltimore, MD (US)

(72) Inventors: Luis Garza, Baltimore, MD (US); Lloyd Miller, Ellicott City, MD (US); Martin Alphonse, Baltimore, MD (US); Mohameed Islam, Baltimore, MD (US)

(21) Appl. No.: 17/916,170

(22) PCT Filed: Mar. 30, 2021
(86) PCT No.: PCT/US2021/024889

§ 371 Date: Sep. 30, 2022

Related U.S. Application Data
(60) Provisional application No. 63/001,674, filed on Mar. 30, 2020.

(51) Int. Cl. A61K 38/05; A61P 31/04; A61P 7/00; A61P 1/00; A61P 17/02; A61P 31/12

(52) U.S. Cl. CPC A61K 38/05; A61P 31/04; A61P 7/00; A61P 1/00; A61P 17/02; A61P 31/12

(57) ABSTRACT

The present invention relates to the field of caspase inhibition. More specifically, the present invention provides compositions and methods utilizing caspase inhibitors to enhance injury repair and to treat bacterial and viral infections. In a specific embodiment, a method for treating a bacterial infection and skin lesions in a patient comprises the step of administering to the patient an effective amount of a caspase inhibitor.

### CROSS-REFERENCE TO RELATED APPLICATIONS

This application claims the benefit of U.S. Provisional Application No. 63/001,674, filed Mar. 30, 2020, which is incorporated herein by reference in its entirety.

### GOVERNMENT SUPPORT CLAUSE

This invention was made with government support under grant no. AR074846, grant no. AR073665, and grant no. AR069502, awarded by the National Institutes of Health. The government has certain rights in the invention.

### FIELD OF THE INVENTION

The present invention relates to the field of caspase inhibition. More specifically, the present invention provides compositions and methods utilizing caspase inhibitors to enhance injury repair and to treat bacterial and viral infections.

### INCORPORATION-BY-REFERENCE OF MATERIAL SUBMITTED ELECTRONICALLY

This application contains a sequence listing. It has been submitted electronically via EFS-Web as an ASCII text file entitled “P16005-02_ST25.txt.” The sequence listing is 1,490 bytes in size, and was created on Mar. 30, 2021. It is hereby incorporated by reference in its entirety.

### BACKGROUND OF THE INVENTION

Antibiotic-resistant bacterial infections have emerged as a major global health crisis, leading to difficult-to-treat infections with high morbidity, mortality and substantial economic burden (1). In an era of a declining antimicrobial pipeline, there is an unmet clinical need to develop host-directed therapies that engage host immune responses as an alternative approach to combat these bacterial infections (2). In particular, community-acquired methicillin-resistant Staphylococcus aureus (CA-MRSA) causes severe skin and soft tissue infections (SSTI) as well as invasive infections (e.g., cellulitis, pneumonia, endocarditis, osteomyelitis and sepsis) in otherwise healthy individuals (3). SSTI are primarily caused by S. aureus (including CA-MRSA) (as well as other bacteria, including Streptococcus pyogenes and Pseudomonas aeruginosa) and result in over 14 million outpatient and emergency room visits and greater than 750,000 hospital admissions annually in the United States (4). This corresponds to an incidence of 48.5 SSTI per 1000 patient-years, which is greater than the incidence of urinary tract infections and pneumonia (4).

To therapeutically target CA-MRSA SSTI, the neutrophilic abscess comprised of myeloid cells (particularly neutrophils as well as monocytes and macrophages) is a key host defense response that walls off the pathogen, prevents the invasive spread and promotes bacterial clearance (5). The important role of neutrophils, monocytes and macrophages in host defense is further supported by the existence of S. aureus pore-forming toxins (i.e., α-toxin, Panton-Valentine Leukocidin [PVL], LukED, γ-hemolysin and LukAB) that mediate cell death of myeloid cells to evade their host defense functions of phagocytosis and bacterial killing (5). Although there are efforts to directly target the neutralization of S. aureus toxins through vaccines and small peptides and molecules (5), we postulated that a counteractive approach that promoted myeloid cell survival would preserve their immune function and provide a therapeutic benefit. Three types of cell death have been implicated to occur during S. aureus infections, including: (1) pyroptosis, an inflammasome dependent inflammatory cell death typically triggered by the nucleotide-binding domain, leucine-rich repeat, family pyrin domain containing 3 [NLRP3]/apoptosis-associated speck-like protein containing a caspase recruitment domain [ASC] inflammasome, which activates caspase-1 or -11 processing of pro-IL-1β to mature IL-1β and Gasdermin D-induced cell membrane pores that result in cell death (as seen during S. aureus challenge in vitro (6-8) and in vivo (9-11)); (2) apoptosis, a non-inflammatory type of programmed cell death mediated by caspases 8 or 9 activation of executioner caspases 3 and 7 (as seen during S. aureus challenge in vitro and in vivo (12-14)); and (3) necroptosis, an inflammatory cell death mediated by death receptor activation (i.e., following binding of TNF, Fas/CD95 and TNF-related apoptosis-inducing ligand [TRAIL]), which triggers receptor-interacting protein (RIP) kinase 1 (RIPK1)/RIPK3/mixed lineage kinase domain-like (MLKL), (as seen during S. aureus challenge in vitro and in vivo (15, 16)).

Quinoline-val-asp-difluorophenoxymethyl ketone (Q-VD-OPH) is a pan-caspase inhibitor that covalently binds and irreversibly blocks multiples caspases (caspases 1, 3 and 7-12) and is cell permeable and non-toxic in vivo (17, 18). Q-VD-OPH inhibits apoptosis in multiple preclinical models of non-infectious injury and viral infection (19-22) and can inhibit or induce necroptosis associated with cerebral ischemia or hepatitis C viral infection, respectively (21, 22). Q-VD-OPH also blocks caspases 1 and 11 (23, 24), and thus might have an effect on pyroptosis. Therefore, we set out to determine whether Q-VD-OPH had therapeutic efficacy via differential mechanistic effects on pyroptosis, apoptosis and necroptosis in a preclinical mouse model of CA-MRSA skin infection.

### SUMMARY OF THE INVENTION

The present invention is based, at least in part, on the discovery that caspase inhibitors can be used to enhance regeneration and repair after skin or gut injury, treat bacterial infections and skin lesions, and to treat viral infections.

In one aspect, the present invention can be used to treat bacterial infections and skin lesions. Indeed, the present invention provides caspase inhibitors as an alternative to antibiotics by engaging the host immune response to promote clearance of bacterial infections. In the U.S., there are over 14 million outpatient/ER visits and 750,000 hospital admissions per year. Also, there are 2 million people who suffer from invasive antibiotic-resistant infections with 23,000 deaths per year in the U.S. alone. The use of caspase inhibitors in the present invention is ground-breaking as it could combat the bacterial infection as an alternative to antibiotic therapy or used in conjunction with antibiotic therapy. This will improve patient outcomes and prevent the spread of antibiotic resistance. As described in Example 2 and the Figures, a caspase inhibitor was used to decrease bacterial burden and skin lesion sizes against a Staphylococcus aureus skin infection, a group A Streptococcus (Streptococcus pyogenes) skin infection and a Pseudomonas aeruginosa skin infection.

Based on the embodiments and experiments described herein, in another aspect, the present invention can be useful to treat viral infections. In a specific embodiment, the compositions and methods of the present invention can be used to treat COVID19. The clinical features of COVID19 include organ damage (LDH, LFTs, troponin), acute respiratory distress syndrome (ARDS) and sepsis. In severe cases, the immunologic features include high C-Reactive Protein (CRP), high cytokines (IL-2R, IL-6, IL-10, TNF), low lymphocytes (CD4 and CD8) and low INF-γ (Chen et al. JCI 2020). The goal of therapy in severe cases includes decrease viral load, decrease bacterial load, decrease organ damage, enhance organ recovery and coagulation control. Immunological goals include increased antiviral/antimicrobial immunity, decrease apoptosis, increase tissue regeneration and modulate/decrease hyperactive immune response. The compositions and methods of the present invention that utilize caspase inhibitors can be used to treat viral infections including COVID19. Thus, in one embodiment, a method for treating COVID19 in a patient comprises the step of administering a caspase inhibitor. In a specific embodiment, the caspase inhibitor comprises Q-VD-OPh. In another embodiment, the method further comprises administering a TLR3 agonist.

In another aspect, the caspase inhibitors can be used to enhance regeneration and repair after skin or gut injury. In specific embodiments, the compositions and methods of the present invention can be used to treat scarring, wounds or any type of temporary injury to skin or gut. More specifically, caspase inhibitors can be used after any injury to the skin or gut—because of surgery or from disease—including major surgery of the gut, skin surgery or burns. Other examples include, but are not limited to, healing after spontaneous or iatrogenic bowel perforation, especially in the context of sepsis. Also included are the cosmetic uses of caspase inhibition in combination with dsRNA to promote antiaging and regeneration. In particular embodiments, caspase inhibitors are used in combination with TLR3 agonists. The embodiments disclosed in Garza et al., U.S. Pat. No. 10,105,305, issued Oct. 13, 2018, is hereby incorporated by reference in its entirety.

In several embodiments, the present invention provides methods and compositions useful for stimulating hair follicle neogenesis. In particular embodiments, the methods and compositions of the present invention are useful to stimulating wound-induced hair neogenesis (WIHN). In one embodiment, a method for stimulating hair follicle neogenesis in a subject comprises the step of administering to the subject an effective amount of a caspase inhibitor, optionally in combination with a TLR3 agonist. In certain embodiments, the TLR3 agonist is a double stranded RNA (dsRNA). In a specific embodiment, the subject has alopecia. In another embodiment, the subject is bald. In a further embodiment, the subject has a wound. The present invention also provides a method for treating a scar in a subject comprising the step of administering to the subject an effective amount of a caspase inhibitor, optionally in combination with a TLR3 agonist.

In particular embodiments, the caspase inhibitor and/or TLR3 agonist is administered directly to a site on the subject that requires hair follicle neogenesis. In a specific embodiment, the caspase inhibitor and/or TLR3 agonist is administered topically. In another embodiment, the caspase inhibitor and/or TLR3 agonist is administered by injection. In certain embodiments, the TLR3 agonist is Polyinosinic:polycytidylic acid (Poly I:C). The TLR3 agonist can also be Hiltonol® or Ampligen®. In another embodiment, the TLR3 agonist comprises IPH3102.

The present invention also provides for the use of caspase inhibitor and/or TLR3 agonists (e.g., dsRNA) as a direct means of stimulating hair neogenesis topically, either as a superficial injection, topical cream or similar method. The compositions of the present invention can also be used in a method to treat removed cells to enhance their ability for regeneration and hair follicle neogenesis, and then implant such cells into a subject. The compositions of the present invention can also be used to activate keratinocytes for use in drug screens to identify compounds that enhance or inhibit the ability for hair neogenesis, and specifically, WNT pathway activation.

In yet another aspect, the present invention provides methods and compositions useful for treating common male pattern hair loss. In one embodiment, a method for treating common male pattern hair loss in a subject comprises the step of administering to the subject an effective amount of a caspase inhibitor, optionally in combination with a TLR3 agonist. In certain embodiments, the TLR3 agonist is a double stranded RNA (dsRNA). In particular embodiments, the caspase inhibitor and/or TLR3 agonist is administered directly to the site of hair loss on the subject. In specific embodiments, the caspase inhibitor and/or TLR3 agonist is administered topically. In an alternative embodiment, the caspase inhibitor and/or TLR3 agonist is administered by injection. The caspase inhibitor and/or TLR3 agonists can be applied locally or also to cultured cells (autologous or allogeneic) ex vivo that are then administered to the patient. In particular embodiments, the TLR3 agonist is Polyinosinic:polycytidylic acid (Poly I:C). In other embodiments, hair follicle neogenesis can be stimulated using LL37 alone or in combination with a TLR3 agonist. In addition, common male pattern hair loss can be treated using LL37 alone or in combination with a TLR3 agonist.

The present invention also provides compositions for carrying out the methods described herein. In particular, the present invention provides a composition comprising a TLR3 agonist and a pharmaceutical carrier. In certain embodiments, the TLR3 agonist is a double stranded RNA (dsRNA). In certain embodiments, wherein the TLR3 agonist is Polyinosinic:polycytidylic acid (Poly I:C). In yet another embodiment, a composition comprises LL-37. In particular embodiments, a composition comprises a dsRNA and LL-37.

### BRIEF DESCRIPTION OF THE FIGURES

FIG. 1A-1J. Q-VD-OPH has a therapeutic effect against a S. aureus skin infection in mice. Mice were either untreated, treated with a vehicle or a single dose of the pan-caspase inhibitor Q-VD-OPH (20 mg/kg i.p.) (n=8-10 mice/group) at 4 hours following S. aureus i.d. skin inoculation. FIG. 1A: Representative digital photographic images. FIG. 1B: Representative in vivo bioluminescence imaging (BLI). FIG. 1C: Mean lesion size (mm) SEM. FIG. 1D: Mean in vivo BLI signals (photons/s)±SEM (log scale). FIG. 1E: Ex vivo CFU (median±ICR) on day 3. FIG. 1F: Representative H&E stained histological sections obtained from untreated and Q-VD-OPH treated mice obtained on day 3. Scale bars=560 μm. Horizontal bracket s=width of dermonecrosis. FIG. 1G: Representative H&E stained histological sections obtained from untreated and Q-VD-OPH treated mice obtained on day 3. Scale bars=240 μm. Black arrowheads denote peripheral edges of the bacterial band length. FIG. 1H: Dermonecrosis width (mm) (median and ICR). FIG. 1I: Abscess area (cm²) (median and ICR). FIG. 1J: Bacterial band length (mm) (median and ICR). *P<0.05, Q-VD-OPH treated mice versus untreated or vehicle-treated mice, as calculated by a 2-way ANOVA multiple comparisons test with adjusted with the Bonferroni correction (FIG. 1C, 1D) or a 2-tailed unpaired Student's t test (FIG. 1E, 1H, 1I, 1J). Data are a compilation of at least 2 independent experiments.

FIG. 2A-2H. The therapeutic mechanism of Q-VD-OPH involves increased monocytes and macrophages and occurs independently of IL-113. Mice were untreated or treated with a single dose of Q-VD-OPH (20 mg/kg i.p.) (n=10 mice/group) at 4 hours following S. aureus i.d. skin inoculation. FIG. 2A: Representative flow cytometry plots for monocytes (CD45⁺CD11b⁺CD115⁺ cells), macrophages (CD45⁺CD11b⁺F4/80⁺ cells), neutrophils (CD45⁺CD115⁻CD11b⁺ and LyG^{hi}LyC^{low/int}) isolated from the infected skin on day 1. FIG. 2B: Percentage and absolute numbers of monocytes, macrophages and neutrophils isolated from the infected skin on day 1 (median±ICR). FIG. 2C: Representative flow cytometry histograms of intracellular pro-IL-1β⁺ cellular expression versus isotype control (Ctrl) on day 1. FIG. 2D: Mean fluorescence intensity (MFI) of pro-IL-1β⁺ cells (median and ICR) on day 1. FIG. 2E: Percentage and absolute numbers of pro-IL-1β⁺ neutrophils, monocytes and macrophages untreated and with Q-VD-OPH treatment±SEM on day 1. FIG. 2F: Serum IL-1β (pg/mL)±SEM on days 1 and 3. FIG. 2G: Mean lesion size (mm)±SEM. FIG. 2H: Mean in vivo BLI signals (photons/s)±SEM (log scale). Data for untreated WT mice performed at the same time are shown as a reference (without statistical comparison) (FIG. 2G, 2H). *P<0.05, Q-VD-OPH treated versus untreated WT mice or IL-1β^{-/-} mice, as calculated by a 2-tailed unpaired Student's t test (FIG. 2B, 2D, 2E, 2F) or 2-way ANOVA (FIG. 2G, 2H). ns=not significant. Data are a compilation of at least 2 independent experiments.

FIG. 3A-3J. Q-VD-OPH inhibits ASC speck formation in vivo. ASC-Citrine mice were untreated or treated with a single dose of Q-VD-OPH (20 mg/kg i.p.) (n=5-10 mice/group) at 4 hours following S. aureus i.d. skin inoculation. FIG. 3A: Representative in vivo bioluminescence imaging (BLI) at 6 hours following Q-VD-OPH treatment. FIG. 3B: In vivo BLI signals (photons/s) (median and ICR) (log scale). FIG. 3C: Representative in vivo fluorescence imaging (FLI) of ASC specks. FIG. 3D: Mean in vivo FLI of ASC specks (median and ICR). FIG. 3E: Representative viSNE plots from flow cytometry analysis showing ASC Citrine expression and total ASC specks (PuLSA assay) formation at days 0 and 1±Q-VD-OPH treatment. FIG. 3F: Percentage and absolute numbers of ASC Citrine/live cells (median and ICR). FIG. 3G: Representative total ASC expression and total ASC speck formation, respectively, at days 0 and 1±Q-VD-OPH treatment. FIG. 3H: Percentage and absolute numbers of ASC Specks/ASC Citrine expression (median and ICR). FIG. 3I: Mean lesion size (mm)±SEM. FIG. 3J: Mean in vivo BLI signals (photons/s) SEM (log scale). Data for untreated WT mice performed at the same time are shown as a reference (without statistical comparison) (FIG. 3I, 3J). *P<0.05, Q-VD-OPH treated versus untreated WT mice or ASC^{-/-} mice, as calculated by a 2-tailed unpaired Student's t test (FIG. 3B, 3D, 3F, 3H) or 2-way ANOVA (FIG. 3I, 3J). ns=not significant. Data are a compilation of at least 2 independent experiments.

FIG. 4A-4H. The therapeutic mechanism of Q-VD-OPH does not involve caspases 1 and 11 or Gasdermin-D. Caspase-1^{-/-}, Caspase-11^{-/-}, Caspase-1/11^{-/-} or Gasdermin D^{-/-} mice were untreated or treated with a single dose of Q-VD-OPH (20 mg/kg i.p.) (n=5-10 mice/group) at 4 hours following S. aureus i.d. skin inoculation. FIG. 4A, 4C, 4E, 4G: Mean lesion size (mm)±SEM. FIG. 4B, 4D, 4F, 4H: Mean in vivo BLI signals (photons/s)±SEM (log scale). Data for untreated WT mice performed at the same time are shown as a reference (without statistical comparison) (FIG. 4A-4G). *P<0.05, Q-VD-OPH treated versus untreated Caspase-1^{-/-}, Caspase-11^{-/-}, Caspase-1/11^{-/-} or Gasdermin D^{-/-} mice, as calculated by a 2-way ANOVA. Data are representative of 2 independent experiments.

FIG. 5A-5G. Q-VD-OPH decreases apoptotic neutrophils/monocytes and increases necroptotic macrophages. WT mice were untreated or treated with a single dose of Q-VD-OPH (20 mg/kg i.p.) (n=5 mice/group) at 4 hours following S. aureus i.d. skin inoculation and singe cell suspensions from infected skin on day 1 were evaluated. FIG. 5A: Representative flow cytometry plots of Annexin V⁺ cells (early apoptotic cells) from CD45⁺ cells isolated from the infected skin on day 1. FIG. 5B: Total percentage and absolute numbers of Annexin V⁺ cells (median and ICR). FIG. 5C: Percentage and absolute numbers of Annexin V⁺ neutrophils, monocytes and macrophages (median and ICR). FIG. 5D: Representative flow cytometry histogram of intracellular pMLKL⁺ cellular expression (a marker of necroptosis) versus isotype control (Ctrl). FIG. 5E: Mean fluorescence intensity (MFI) of pMLKL⁺ cells (median and ICR). FIG. 5F: Percentage and absolute numbers of active pMLKL⁺ total cells (median and ICR). FIG. 5G: Percentage of active pMLKL⁺ neutrophils, monocytes and macrophages±SEM. *P<0.05, Q-VD-OPH treated mice versus untreated mice, as calculated by a 2-tailed unpaired Student's t test. ns=not significant. Data are representative of 2 independent experiments.

FIG. 6A-6K. Q-VD-OPH efficacy is dependent upon increased levels and activity of TNF on reducing the bacterial burden in vivo. WT mice, TNF^{-/-}, WT mice±a blocking mAb against TNF or IL-1R/TNF^{-/-} were untreated or treated with a single dose of Q-VD-OPH (20 mg/kg i.p.) (n=5-10 mice/group) at 4 hours following S. aureus i.d. skin inoculation. FIG. 6A: Representative flow cytometry histogram of intracellular TNF⁺ cellular expression versus isotype control (Ctrl) on day 1. FIG. 6B: Mean fluorescence intensity (MFI) of TNF⁺ cells (median and ICR) on day 1. FIG. 6C: Percentage and absolute numbers of TNF⁺ neutrophils, monocytes and macrophages±SEM on day 1. FIG. 6D: Mean serum TNF levels (pg/mL)±SEM on days 1 and 3. FIG. 6E, 6G, 6J: Mean lesion size (mm)±SEM. FIG. 6F, 6H,6K: Mean in vivo BLI signals (photons/s)±SEM (log scale). FIG. 6I: Timeline of administration of anti-TNF blocking mAb (or IgG isotype control mAb [IgG Ctrl] [FIG. 12])±Q-VD-OPH treatment. *P<0.05, Q-VD-OPH treated mice versus untreated mice, as calculated by a 2-tailed unpaired Student's t test (FIG. 6B, 6C, 6D) or 2-way ANOVA (FIG. 6F-6K). ns=not significant. Data are a compilation (FIG. 6A-6D) or representative (FIG. 6E-6K) of 2 independent experiments.

FIG. 7A-7C. Combined caspase 3, 8 and 9 inhibition reduced apoptosis and combined caspase 1, 8 and 11 inhibition induced TNF production, similar to Q-VD-OPH in vitro. BMDMs were incubated with caspase 1, 3, 8, 9 or 11 inhibitors (Z-WEHD-FMK [100 μM], Z-DQMD-FMK [10 μM], Z-IETD-FMK [100 μM], Z-LEHD-FMK [100 μM], wadelolactone [20 μM], respectively), caspase 3, 8 and 9 inhibitors in combination, caspase 1, 8 and 11 inhibitors in combination, Q-VD-OPH (10 μg/mL), Emricasan (9 μg/mL) or no treatment (None) and live S. aureus for 6 hours with gentamicin added after the first hour of culture. Early apoptosis marker (Annexin-V⁺) and intracellular levels of TNF were assayed. FIG. 7A: Representative flow histograms of Annexin-V⁺ CD11b⁺ BMDMs. FIG. 7B: Percentage of Annexin-V⁺CD11b⁺ BMDMs±SEM. FIG. 7C: Representative flow histograms of TNF expression verses isotype control (Ctrl) of CD11b⁺ BMDMs. (D) Percentage of TNF⁺ cells CD11b⁺ BMDMs±SEM. *P<0.05, each caspase inhibitor or Q-VD-OPH versus no treatment (None), as calculated by a 2-way ANOVA multiple comparisons test adjusted with Bonferroni correction. ns=not significant. Data are a combination of 3 independent in vitro experiments.

FIG. 8A-8I. Q-VD-OPH has efficacy against a S. pyogenes or P. aeruginosa skin infection in mice and mortality following bacterial dissemination in mice. Mice were either untreated or treated a single dose of the pan-caspase inhibitor Q-VD-OPH (20 mg/kg i.p.) (n=10 mice/group) at 4 hours following S. pyogenes or P. aeruginosa i.d. skin inoculation. FIG. 8A: Representative digital photographic images of S. pyogenes. FIG. 8B: Representative in vivo bioluminescence imaging (BLI) of S. pyogenes. FIG. 8C: Mean lesion size (mm)±SEM of S. pyogenes. FIG. 8D: Mean in vivo BLI signals (photons/s)±SEM (log scale) of S. pyogenes. FIG. 8E: Representative digital photographic images of P. aeruginosa. FIG. 8F: Representative in vivo bioluminescence imaging (BLI) of P. aeruginosa. FIG. 8G: Mean lesion size (mm)±SEM of P. aeruginosa. FIG. 8H: Mean in vivo BLI signals (photons/s)±SEM (log scale) of P. aeruginosa. FIG. 8I: Kaplan-Meier survival curves for untreated and Q-VD-OPH treated mice following P. aeruginosa i.d. inoculation. *P<0.05, Q-VD-OPH treated mice versus untreated mice, as calculated by a 2-way ANOVA (FIG. 8C, 8D, 8G, 8H) or a Log-rank Mantel-Cox test (FIG. 8I). Data are a compilation of 2 independent experiments.

FIG. 9A-9C. Q-VD-OPH does not have direct in vitro antibacterial activity against S. aureus. Bacterial broth cultures were incubated with vehicle (Veh) or various concentrations of Q-VD-OPH (10 μg/mL, 100 μg/mL, and 1,000 μg/mL). The bacterial growth (OD₆₀₀) and metabolic activity as measured by bioluminescence (Lum) was measured (n=3) in triplicate for 10 hours with measurements recorded at 20-minute intervals. Bacterial growth (OD₆₀₀) (FIG. 9A), luminescence (Lum) (FIG. 9B) and CFU (median±ICR) at 10 hours (at the end of the experiment) (FIG. 9C) in S. aureus broth cultures incubated with Q-VD-OPH or Veh. The curves are represented as smooth lines±SEM. ns=non-significant, as calculated by 2-way ANOVA multiple comparisons test adjusted with the Bonferroni correction. Data are representative of 3 independent experiments, with all of the Q-VD-OPH experimental and Veh groups performed in triplicate.

FIG. 10A-10D. Gating strategy for cell populations. FIG. 10A: Representative flow plots for the CD45⁺ population. FIG. 10B-10D: Representative flow plots showing the gating strategy for monocytes (CD45⁺CD11b⁺CD115⁺), macrophages (CD45⁺CD11b⁺F4/80⁺) and neutrophils (CD45⁺CD11b⁺Ly6G^{hi} Ly6C^{int/low}), respectively.

FIG. 11A-11E. viSNE analysis of population cluster in ASC Citrine expression. ASC Citrine mice were treated with or without a single dose of Q-VD-OPH 20 mg/kg (i.p) during the inoculation with 3×10⁷ CFU/100 μL of CA-MRSA (i.d.). FIG. 11A: Representative flow plots and viSNE plot showing the live gate and clusters (C1, C3-05) of cell populations. FIG. 11B-11E: Representative viSNE plots showing labeling of neutrophils (CD45⁺CD11b⁺Ly6G^{hi} Ly6C^{int/low}), Langerhans cells (CD45⁺CD207⁺CD103⁻), monocyte-derived dendritic cells (MDDCs) (CD45⁺CD11c⁺CD115⁺), and monocytes (CD45⁺CD11b⁺CD115⁺), respectively.

FIG. 12A-12B. Isotype control treatment for FIG. 6I-K with an anti-TNF blocking mAb in a mouse model of CA-MRSA treated with pan-caspase inhibitor Q-VD-OPH. WT mice (n=5 mice/group) were treated with an IgG isotype control mAb (mAb isotype control for the anti-TNF blocking mAb in FIG. 6I-K)±a single dose of Q-VD-OPH 20 mg/kg (i.p) 4 hours after inoculation with 3×10⁷ CFU/100 μL of CA-MRSA. FIG. 12A-12B: Graphical representation of the lesion size and BLI of mice with IgG Isotype Ctrl (α-IgG Ctrl) with or without Q-VD-OPH treatment overtime, respectively. *P<0.05, Q-VD-OPH treated mice versus untreated or vehicle-treated mice, as calculated by 2-way ANOVA.

FIG. 13A-13C. Q-VD-OPH treatment in TNF^{-/-} reduces the infiltration of neutrophils. TNF^{-/-} mice were untreated (n=7) or treated with a single dose of Q-VD-OPH (20 mg/kg i.p.) (n=8) at 4 hours following S. aureus i.d. skin inoculation. FIG. 13A: Representative flow plots for the CD45⁺ population and viSNE plot showing monocytes, macrophages and neutrophils. FIG. 13B: Representative viSNE plots for monocytes (CD45⁺CD11b⁺CD115⁺ cells), macrophages (CD45⁺CD11b⁺F4/80⁺ cells), neutrophils (CD45⁺CD115⁻CD11b⁺ and LyG^{hi}LyC^{low/int}) isolated from the infected skin on day 1. FIG. 13C: Percentage and absolute numbers of monocytes, macrophages and neutrophils isolated from the infected skin on day 1 (median±ICR). *P<0.05, Q-VD-OPH treated mice versus untreated mice, as calculated by a 2-tailed unpaired Student's t test. ns=not significant. Data are representative of 2 independent experiments.

FIG. 14A-14G. Q-VD-OPH decreases apoptotic neutrophils and increases necroptotic neutrophils and macrophages in TNF^{-/-} mice. TNF^{-/-} mice were untreated (n=7) or treated with a single dose of Q-VD-OPH (20 mg/kg i.p.) (n=8) mice at 4 hours following S. aureus i.d. skin inoculation and singe cell suspensions from infected skin on day 1 were evaluated. FIG. 14A: Representative viSNE plots of Annexin V⁺ cells (early apoptotic cells) from CD45⁺ cells isolated from the infected skin on day 1. FIG. 14B: Total percentage and absolute numbers of Annexin V⁺ cells (median and ICR). FIG. 14C: Percentage and absolute numbers of Annexin V⁺ neutrophils, monocytes and macrophages (median and ICR). FIG. 14D: Representative viSNE plots of intracellular pMLKL⁺ cellular expression. FIG. 14E: Mean fluorescence intensity (MFI) of pMLKL⁺ cells (median and ICR). FIG. 14F: Percentage and absolute numbers of active pMLKL⁺ total cells (median and ICR). FIG. 14G: Percentage of active pMLKL⁺ neutrophils, monocytes and macrophages±SEM. *P<0.05, Q-VD-OPH treated mice versus untreated mice, as calculated by a 2-tailed unpaired Student's t test. ns=not significant. Data are representative of 2 independent experiments.

FIG. 15. Q-VD-OPH increases necroptosis and decreases apoptosis in S. aureus-infected skin from WT but not TNF^{-/-} mice. WT untreated and TNF^{-/-} mice were untreated or treated with a single dose of Q-VD-OPH (20 mg/kg i.p.) (n=3 mice/group) at 4 hours following S. aureus i.d. skin inoculation and infected skin on day 1 was evaluated for expression of pMLKL, MLKL, ICAD, and β-actin by western blot analysis.

FIG. 16A-16D. Combined caspase 3, 8 and 9 inhibition reduced apoptosis and combined caspase 1, 8 and 11 inhibition induced TNF production, similar to Q-VD-OPH in vitro. Neutrophils were incubated with caspase 1, 3, 8, 9 or 11 inhibitors (Z-WEHD-FMK [100 μM], Z-DQMD-FMK [10 μM], Z-IETD-FMK [100 μM], Z-LEHD-FMK [100 μM], wadelolactone [20 μM], respectively), caspase 3, 8 and 9 inhibitors in combination, caspase 1, 8 and 11 inhibitors in combination, Q-VD-OPH (10 μg/mL), Emricasan (9 μg/mL) or no treatment (None) and live S. aureus for 6 hours with gentamicin added after the first hour of culture. Early apoptosis marker (Annexin-V⁺) and intracellular levels of TNF were assayed. FIG. 16A: Representative flow histograms of Annexin-V⁺CD11b⁺Ly6G^{hi} neutrophils. FIG. 16B: Percentage of Annexin-V⁺CD11b⁺Ly6G^{hi} neutrophils±SEM. FIG. 16C: Representative flow histograms of TNF expression of Annexin-V⁺CD11b⁺Ly6G^{hi} neutrophils. FIG. 16D: Percentage of TNF⁺ cells Annexin-V⁺CD11b⁺Ly6G^{hi}±SEM. *P<0.05, each caspase inhibitor group or Q-VD-OPH versus no treatment (None), as calculated by a 2-way ANOVA multiple comparisons test adjusted with Bonferroni correction. ns=not significant. Data are a combination of 3 independent in vitro experiments.

FIG. 17A-17H. Caspases 3 and 9 inhibition reduced apoptosis and caspase 1, 8 and 11 inhibition independently does induce TNF production in vitro. BMDMs or Neutrophils were incubated with caspase 1, 3, 8, 9 or 11 inhibitors (Z-WEHD-FMK [100 μM], Z-DQMD-FMK [10 μM], Z-IETD-FMK [100 μM], Z-LEHD-FMK [100 μM], wadelolactone [20 μM], respectively), or no treatment (None) and live S. aureus for 6 hours with gentamicin added after the first hour of culture. Early apoptosis marker (Annexin-V⁺) and intracellular levels of TNF were assayed. FIG. 17A: Representative flow histograms of Annexin-V⁺ CD11b⁺ BMDMs. FIG. 17B: Percentage of Annexin-V⁺CD11b⁺ BMDMs±SEM. FIG. 17C: Representative flow histograms of TNF expression verses isotype control (Ctrl) of CD11b⁺ BMDMs. FIG. 17D: Percentage of TNF⁺ cells CD11b⁺ BMDMs±SEM. FIG. 17E: Representative flow histograms of Annexin-V⁺CD11b⁺Ly6G^{hi} neutrophils. FIG. 17F: Percentage of Annexin-V⁺CD11b⁺Ly6G^{hi} neutrophils±SEM. FIG. 17G: Representative flow histograms of TNF expression of Annexin-V⁺CD11b⁺Ly6G^{hi} neutrophils. FIG. 17H: Percentage of TNF⁺ cells Annexin-V⁺CD11b⁺Ly6G^{hi}±SEM. *P<0.05, each caspase inhibitor group or Q-VD-OPH versus no treatment (None), as calculated by a 2-way ANOVA multiple comparisons test adjusted with Bonferroni correction. ns=not significant. Data are a combination of 3 independent in vitro experiments.

FIG. 18A-18C. Gating strategy for cell populations for BMDMs and neutrophils. FIG. 18A, 18B: Representative flow plots showing the gating strategy for CD11b⁺ BMDMs and Annexin V⁺ gating, respectively. FIG. 18C: Representative flow plots for the mouse bone marrow CD11b⁺Ly6G^{hi} neutrophils (PMN).

FIG. 19A-19B. Viability of BMDMs and neutrophils in the in vitro experiments in FIG. 7 and FIGS. 16 and 17. BMDMs or neutrophils (PMN) were incubated with caspase 1, 3, 8, 9 and 11 inhibitors (Z-WEHD-FMK [100 μM], Z-DQMD-FMK [10 μM], Z-IETD-FMK [100 μM], Z-LEHD-FMK [100 μM], wadelolactone [20 μM], respectively), Q-VD-OPH (10 μg/mL), Emricasan (9 μg/ml) or no treatment (none) and live S. aureus for 6 hours with gentamicin added after the first hour of culture. Viability of cells are measured as Zombie UV negative cells by flow cytometry. FIG. 19A: Mean live cells±SEM of BMDMs. FIG. 19B: Mean live cells±SEM of PMNs. ns=not significant. Each caspase inhibitor or Q-VD-OPH versus no treatment (None), as calculated by a 2-tailed unpaired Student's t test. Data are a combination of 3 independent stimulation experiments.

FIG. 20A-20B. Q-VD-OPH increases necroptosis in vitro in BMDMs and PMNs from WT mice. BMDMs or neutrophils from WT mice were treated with Q-VD-OPH (10 μg/mL), or positive control for necroptosis [SMAC mimetic (100 nm)+TNF (20 ng/ml)+zVAD (20 μM) for 8 hours] or no treatment (None) and live S. aureus for 6 hours with gentamicin added after the first hour of culture. Protein expression for pMLKL, MLKL, and β-actin were performed from 3 wells combined together from cultured BMDMs (FIG. 20A) and PMNs (FIG. 20B), respectively, by western blot analysis.

FIG. 21A-21D. Q-VD-OPH does not have direct in vitro antibacterial activity against S. pyogenes and P. aeruginosa. Bacterial broth cultures were incubated with vehicle (Veh) or various concentrations of Q-VD-OPH (10 μg/mL, 100 μg/mL, and 1,000 μg/mL). The bacterial growth (OD₆₀₀) and metabolic activity as measured by bioluminescence (Lum) was measured (n=3) in triplicate for 10 hours with measurements recorded at 20 minute intervals. Bacterial growth (OD₆₀₀) (FIG. 21A) and luminescence (Lum) (FIG. 21B) in S. pyogenes broth cultures incubated with Q-VD-OPH or Veh. Bacterial growth (OD₆₀₀) (FIG. 21C) and luminescence (Lum) (FIG. 21D) in P. aeruginosa broth cultures incubated with Q-VD-OPH or Veh. The curves are represented as smooth lines±SEM. ns=non-significant, as calculated by 2-way ANOVA multiple comparisons test adjusted with the Bonferroni correction. Data are representative of 3 independent experiments, with all of the Q-VD-OPH experimental and Veh groups performed in triplicate.

FIG. 22A-22E. Q-VD-OPH does not inhibit the activity of S. aureus and S. pyogenes bacterial cysteine proteases. Staphopain A (sspP), Staphopain B (sspB) or Streptopain B (speB) at 1 ng, 10 ng or 100 ng were incubated with or without 10 μg/mL of Q-VD-OPH for 20 hours along with Gelatin or Elastin. FIG. 22A: Protease activity against gelatin±SEM measured in sspP, sspB, and speB (1, 10 and 100 ng) either separately or in combination with Q-VD-OPH (10 μg/mL) for 20 hours. Q-VD-OPH alone (10, 100, 1000 μg/mL), Collagenase (0.4 U/mL) and Collagenase with inhibitor 1,10-Phenanthroline (1 mM) were used for positive and inhibitory controls respectively. FIG. 22B: Protease activity against elastin±SEM measured in sspP, sspB, and speB (1, 10 and 100 ng) either separately or in combination with Q-VD-OPH (10 μg/mL) for 20 hours. Q-VD-OPH alone (10, 100, 1000 μg/mL), Elastase (0.25 U/mL) and Elastase with inhibitor N-methoxysuccinyl-Ala-Ala-Pro-Val-chloromethyl ketone (0.1 mM) were used for positive and inhibitory controls respectively. ns=non-significant, as calculated by a 2-tailed unpaired Student's t test. Data are a combination of 3 independent experiments. Protein blots for (FIG. 22C) sspP, (FIG. 22D) sspB, and (FIG. 22E) speB either separately or in combination with Q-VD-OPH (10 μg/mL) for 2 hours were resolved in PVDF membrane and stained for protein degradation determination by colorimetric protein stain.

FIG. 23A-23G. Trans-species dsRNA sensing signature during skin regeneration; RNase L represses regeneration markers. FIG. 23A: Three-way Venn diagram shows 14 gene overlap present in all of the top 200 genes in microarrays of in vivo wound induced hair neogenesis (WIHN) comparing C57BL/6×FVB×SJL (high regeneration strain) vs. C57BL/6 versus (low regeneration strain), in vivo human clinical trial of subjects treated with a rejuvenation laser, and in vitro human keratinocytes treated with dsRNA/Poly (I:C) as a positive control. The in vitro and in vivo human microarrays contain a total of 49395 annotated transcripts each, and the in vivo murine microarray contains 53145 transcripts. FIG. 23B: Gene ontology analysis of each of the individual top 200 gene lists, highlighting the predominance of OAS family members in each data set. FIG. 23C: Gene ontology terms enriched in the 14 overlapping genes from all three datasets include the upregulation of OAS family genes. Inset graphs show the gene fold expression changes from the original microarray for genes present in that category; green and blue indicate mouse and human respectively. FIG. 23D: In an analysis of the ribonuclease RNase L (downstream and activated by OAS), Venn diagram shows the top 200 overlapping genes in Rnasel^{-/-} mice after wounding (at scab detachment) and human keratinocytes treated with siRNA targeting RNase L. GO categories include multiple developmental pathways. The in vivo microarray data contain 22206 transcripts and the in vitro RNA-seq data contain 33264 transcripts. FIG. 23E-23G Poly (I:C) treatment (10 μg/ml) of RNase L siRNA transfected human keratinocytes induces multiple morphogenesis markers (FIG. 23E; n=3, p<0.05), including WNT7B (green) shown by immunostaining (FIG. 23F) but inhibits differentiation markers (FIG. 23G; n=3, p<0.05).

FIG. 24A-24G. RNase L loss enhances hair follicle regeneration (WIHN). FIG. 24A: Rnasel^{-/-} mice exhibit increased wound induced hair neogenesis (WIHN) with an intact super-increase in the presence of poly (I:C) (confocal scanning laser microscopy, CSLM, images; n=10 each, p<0.0001). In each image, the dash red box indicates the area of hair follicle regeneration. FIG. 24B: Rnasel^{-/-} mice display normal wound closure speed (n=10 vs 4). FIG. 24C: Transepidermal water loss (TEWL) was measured in the center and periphery of healed skin at scab detachment day (WD10) for both wild-type and Rnasel^{-/-} mice. Rnasel^{-/-} mice exhibit dramatically lower TEWL measurements, consistent with a post-wounding improved barrier compared to wild type mice (n=3, p<0.005, p=0001). FIG. 24D: Rnasel^{-/-} mice have greater morphogenesis marker gene expression of Tlr3 (n=3, P<0.01), 116 (n=3, p<0.0001), Wnt7b (n=3, P<0.01), and Edar (n=3, p<0.01) on day of re-epithelialization as measure by qRT-PCR. FIG. 24E: Unwounded skin of Rnasel^{-/-} mice show increased protein expression of stem cell markers Krt5 (green), Krt15 (red) and morphogenesis marker Wnt7b (green) shown by immunofluorescence. FIG. 24F: Gene ontology analysis of Rnasel^{-/-} mice during re-epithelialization (˜10 days post-wounding) show enrichment of IL-1 response, neutrophils, and wound healing pathways. Individual genes corresponding to each category are shown in green. FIG. 24G: At 3 days post-wounding, Rnasel^{-/-} mice recruit significantly more neutrophils in the wound bed. Ly6G (green) is a neutrophil marker and H4Cit (red) is a marker for citrullinated histones released from neutrophils during NETosis. Nuclear staining was performed using DAPI (blue). The white dashed line signifies the dorsal edge of the wound bed. White scale bar=100 μm.

FIG. 25A-25K. RNase L suppresses IL36 expression, which is required for and promotes WIHN. FIG. 25A: Gene ontology analysis of the top 100 genes in a microarray of high regenerating outbred wild-type strain mice (C57BL/6×FVB×SJL) compared to the lower regenerating wild type C57BL/6 and the top 100 proteins found in the center (high regenerating) versus the edge (low regenerating) areas of the wound show a common signature for IL-1 family member Il36α (red) and neutrophil granule proteins (orange). FIG. 25B: Heat map analyses from (a) show Il-1 family members are enriched in the High regeneration mice and Center of the wound, particularly Il36 family members (red). Neutrophil granule proteins (orange), known to proteolytically cleave and activate Il36 proteins, are also enriched. FIG. 25C: Wounded tissue from Rnasel^{-/-} mice reveal elevated IL36α protein as shown by western blot. FIG. 25D: Both unwounded and wounded skin show increased expression of IL36α (green) in Rnasel^{-/-} mice. IL36α expression peaks in both wild-type and Rnasel^{-/-} mice at 3 days post-wounding. FIG. 25E: Keratinocytes harvested and cultured from Rnasel^{-/-} mice actively secrete more IL36α compared to wild-type controls, as shown by western blot. FIG. 25F: Injection of 50 ng of recombinant IL36α protein underneath the scab at WD7 promotes WIHN (n=3, P<0.01). FIG. 25G: Histology of (E) comparing vehicle or rmIL36α treated mice skin sections. The neogenic hair follicles (purple) are shown aggregated at the center of the scar. FIG. 25H: Il36r^{-/-} mice fail to regenerate hair follicles and are not responsive to poly (I:C) (n=3, p<0.01). FIG. 25I: Rnasel^{-/-}/Il36r^{-/-} mice lose the ability to regenerate hair follicles compared to Rnasel^{-/-} mice (n=7, P<0.0001). FIG. 25J: siRNA Knockdown of IL36α and RNase L in human keratinocytes abrogates the increases of both WNT7B and IL6 morphogenesis markers, compared to RNase L siRNA alone (n=3 each, P<0.05). FIG. 25K: Treatment of human keratinocytes with recombinant IL36α increases WNT7B mRNA expression (n=3, P<0.05) as quantified by qRT-PCR.

FIG. 26A-26L. Caspases, known downstream mediators of RNase L, restrain IL-36 release and regeneration. FIG. 26A: Bioinformatics analysis was performed on the top 200 genes unregulated in vivo in Rnasel^{-/-} mice and the top 200 genes in vitro in human keratinocytes treated with siRNA targeting RNase L. Gene ontology analysis was performed on the top 50 shared genes and revealed biological processes for vesicle trafficking, proteases, and an apoptosis signature. FIG. 26B: Using the gene list from (FIG. 26A), we performed a siRNA screen in mouse keratinocytes and identified caspase-1 as a consistent target whose loss induced IL-36α protein expression. FIG. 26C: siRNA knockdown of caspase-1 in mouse keratinocytes leads to elevated secretion of IL36α. FIG. 26D: Schematic of the pan-caspase inhibitor Q-VD-OPh intraperitoneal injection (1.33 mM) of mice wounded to measure WIHN. IP injections were done one day before and ten days after wounding C57BL/J6 mice with 1.25 cm×1.25 cm square wounds. Mice were then sacrificed at wound day 21 to measure WIHN. FIG. 26E: QV-D-OPh induces elevation of neutrophils in unwounded skin shown by t-SNE analysis and quantification (n=3, P<0.05). FIG. 26F: QV-D-OPh for 48 hrs induces IL36α mRNA in cultured mouse keratinocytes (n=6, ****P<0.0001, **P=0.0012). FIG. 26G: QV-D-OPh induces IL-36α protein without inhibition of the receptor antagonist IL-36rn in whole-cell lysates of mouse epithelial keratinocytes (P=0.0251 by two-way ANOVA, n=3) FIG. 26H. QV-D-OPh (as in FIG. 26D) promotes WIHN compared to vehicle as shown by CSLM (n=3 versus 4, P<0.001). FIG. 26I: Histology of (FIG. 26H) comparing vehicle to Q-VD-OPh treated mouse skin sections. The neogenic hair follicles (purple) are shown aggregated at the center of the scar. Black scale bar=100 μm. FIG. 26J: Immunostaining of re-epithelialized wounded tissue shows elevated IL36α in Q-VD-OPh treated mice. FIG. 26K: Il36r^{-/-} mice do not respond to Q-VD-OPh treatment and are unable to regenerate hair follicles after wounding (n=4, n.s.=not significant). FIG. 26L: Histology of WIHN scars from (FIG. 26K). Black scale bar=200 μm.

FIG. 27A-27H. Caspase inhibition promotes gut regeneration in DSS-treated mice. FIG. 27A: Schematic of Q-VD-OPh intraperitoneal injection (1.33 mM) in 4% DSS treated C57BL/J6 mice. FIG. 27B: Q-VD-OPh treatment is able to rescue weight loss in a DSS-induced colitis model. After 6 days of DSS treatment, mice begin exhibiting drastic changes in QVD versus vehicle treated mice (n=4, P<0.05). FIG. 27C: Q-VD-OPh-treated mice have longer colon lengths compared to vehicle after DSS-induced damage (n=4, P=0.0203). Colon lengths were normalized to their starting body weight. FIG. 27D: Q-VD-OPh induces IL36α expression (green) in the colon as it does in skin FIG. 27E. Q-VD-OPh does not rescue weight loss in Il36r^{-/-} mice (n=3 versus 4). FIG. 27F: Q-VD-OPh does not rescue gut shortening after DSS treatment in in Il36r^{-/-} mice (n=3 versus 4). FIG. 27G: Histology of colon sections in (FIG. 26C) and (FIG. 27F) showing gross improvement of tissue after pan-caspase inhibition in wild-type, but not Il36r^{-/-} mice. FIG. 27H: Model of regeneration highlighting RNase L as a regeneration suppressor that acts through caspase stimulation and IL-36 suppression.

FIG. 28A-28B. Proteomics analysis of WT versus Rnasel^{-/-} keratinocytes. FIG. 28A: The top 100 proteins elevated in Rnasel^{-/-} keratinocytes compared to wild type keratinocytes. FIG. 28B: The genes in (FIG. 28A) were analyzed using gene ontology and show significant upregulation of biological processes for developmental and morphogenesis pathways.

FIG. 29A-29D. circRNA analysis of WT versus Rnasel^{-/-} mice. FIG. 29A-30B: Bioinformatic analysis of circRNA abundance by type (exonic, intragenic, intronic) in unwounded skin extracted from wild type (FIG. 29A) and Rnasel^{-/-} (FIG. 29B) mice shows a majority of exonic regions in both stains. FIG. 29C: Gene ontology analysis of top 100 circRNAs (GFOLD<0.5) in Rnasel^{-/-} versus wild-type reveals unregulated biological processes for development, morphogenesis and Wnt signaling. P-values calculated using a non-corrected Fisher's exact test. FIG. 29D: Preferential upregulation of circRNA abundance (E-FOLD>0.5) rather than downregulation in Rnasel^{-/-} versus wild-type mice via circRNA-seq from a total of 4300 transcripts.

FIG. 30A-30C. RNase L loss in mouse epithelial keratinocytes induces morphogenesis markers without affecting interferon levels. siRNA-mediated RNase L loss in mouse epithelial keratinocytes induced multiple morphogenesis markers (FIG. 30A), while not affecting levels of interferons (FIG. 30B), with and without poly (LC) treatment (10 μg/ml, 48 hrs) (n=3, P<0.05). FIG. 30C: Il36α and Casp1 levels were elevated after siRNA-mediated RNase L loss, and boosted with poly (I:C) treatment (10 μg/ml, 48 hrs).

FIG. 31. Rnasel^{-/-} mice have normal wound closure kinetics. Grossly, Rnasel^{-/-} mice display normal wound closure speed. Images from wound days 0 and 8 (n=10 vs 4).

FIG. 32. Rnasel^{-/-} mice have a thinner hypodermal layer. Hematoxylin and eosin staining of unwounded tissue from wild type and Rnasel^{-/-} mice show that Rnasel^{-/-} mice have a thinner hypodermis compared to wild-type mice (n=3, P<0.05). Black scale bar=100 μm.

FIG. 33A-33C. Rnasel^{-/-} mice have elevated levels of Retinoic Acid in skin. FIG. 33A: Quantitation and analysis of retinoic acid (RA) levels demonstrate more RA is present in Rnasel^{-/-} compared to wild-type mice in unwounded skin (n=4, P<0.05). Measurements were acquired via LC-MS. FIG. 33B: Quantitation and analysis of retinol (ROL) levels in unwounded skin of wild type and Rnasel^{-/-} mice does not show a similar change (n=4, P=n.s.). FIG. 33C: Quantitation and analysis of retinyl ester (RE) levels in unwounded skin of wild-type and Rnasel^{-/-} mice is not significant (n=4, p<n.s.).

FIG. 34. Rnasel^{-/-} mice have elevated levels of endogenous U1 snRNA. U1 snRNA was measured in both unwounded and healed wounds of wild type and Rnasel^{-/-} mice via qRT-PCR using custom TaqMan probes. Compared to wild type mice, Rnasel^{-/-} mice express significantly higher U1 snRNA than wild-type mice (n=3, p<0.05). U6 snRNA was used as a housekeeping control.

FIG. 35A-35B. Nlrp3^{-/-} mice have enhanced WIHN. FIG. 35A: Nlrp3^{-/-} mice exhibit increased wound induced hair neogenesis (WIHN) when compared to wild type age-matched control mice. (CSLM images; n=14 versus 18 each, P=0.0046). FIG. 35B: Chemical inhibition of NLRP3 by MCC950 (100 μM) in human keratinocytes treated with poly (I:C) increased expression of morphogenesis markers.

FIG. 36A-36C. Caspase inhibition enhances IL36α levels in mouse epithelial keratinocytes. FIG. 36A: IL36rn and IL36r expression was not affected by caspase inhibition in mouse epithelia keratinocytes treated with QV-D-OPh for 48 hrs. Tlr3 and inflammatory caspases 1 and 4 were elevated after QV-D-OPh treatment (n=3). FIG. 36B: IL-36α protein levels, but not the receptor antagonist IL-36rn, were increased in whole-cell lysates of mouse epithelial keratinocytes treated with Group 1 caspase inhibitor Z-WEHD-FMK. IL36rn and IL36r expression was not affected by caspase inhibition in mouse epithelial keratinocytes treated with Z-WEHD-FMK for 48 hrs. Tlr3 and inflammatory caspases 1 and 4 are elevated after Z-WEHD-FMK (n=3). FIG. 36C: IL-36α protein levels were increased in whole-cell lysates of mouse epithelial keratinocytes treated with pan caspase inhibitor Emericasan. Results are representative of three independent experiments.

FIG. 37A-37C. Q-VD-OPh treatment in DSS treated mice does not significantly affect inflammation in blood and colon. FIG. 37A: Circulating neutrophils (CD11b+, Ly6G+, Ly6c+) are elevated, while monocytes (CD11b+, Ly6G−, Ly6c++) are reduced in the blood of on day 6 of a 7-day regimen of 4% DSS to induce colitis, indicating inflammation. (n=5 each, P<0.0001 for neutrophils and monocytes). FIG. 37B: One day after removing the mice from DSS treatment (D8), neutrophil (CD11b+, Ly6G+, Ly6c+) monocyte (CD11b+, Ly6G−, Ly6c++), and T cell (CD11b+, CD3+) levels in the blood were unchanged between Q-VD-OPh and vehicle treated mice, suggesting inflammation decreases after removal of DSS, regardless of the presence of Q-VD-OPh. (n=3 versus 4 each, n.s.=not significant). FIG. 37C: Neutrophil (CD11b+, Ly6G+, Ly6c+) and monocyte (CD11b+, Ly6G−, Ly6c++) levels within the colon post 4% DSS treatment (D10) are consistent between vehicle and Q-VDOPh treatment, suggesting Q-VD-OPh treatment is promoting regeneration rather than reducing inflammation.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

Where “about” is used in connection with a number, this can mean the number+/−15%, the number plus 5%, or the number itself without “about.” For example, “about 100” would stand for “from and including 85 to and including 115”. Where “about” is used in connection with numeric ranges, for example “about 1 to about 3”, or “between about one and about three”, preferably the definition of “about” given for a number in the last sentence is applied to each number defining the start and the end of a range separately. In certain embodiments, where “about” is used in connection with any numerical values, the “about” can be deleted.

As used herein, the terms “patient”, “subject” and “subjects” refer to an animal, preferably a mammal including, but not limited to, a non-primate (e.g., a cow, pig, horse, cat, dog, rat, and mouse) and a non-human primates (e.g., a monkey such as a cynomolgous monkey), and more preferably a human. In a specific embodiment, the subject or patient is a human.

As used herein, the term “effective amount” refers to the amount of an agent (e.g., a caspase inhibitor or other agent) which is sufficient to cause the desired effect in the particular context; prevent, reduce or ameliorate the severity, duration and/or progression of a disease or condition or one or more symptoms thereof; ameliorate one or more symptoms of a disease or condition; prevent the advancement of a disease or condition; cause regression of a disease or condition; prevent the recurrence, development, or onset of a disease or condition or one or more symptoms thereof; or enhance or improve the prophylactic or therapeutic effect (s) of another therapy (e.g., prophylactic or therapeutic agent).

### II. Caspase Inhibitors

The compositions and methods of the present invention utilize one or more caspase inhibitors. Specific examples of caspase inhibitors include, but are not limited to, IDN-6556 (Pfizer), IDN-6734 (Pfizer), VX-740 (Pralnacasn, Vertex/Aventis) and VX-765 (Vertex/Aventis). Pralnascan and VX-765 are reversible inhibitors, while IDN-6556 is irreversible. VX-765 is a prodrug that yields the drug VRT-043198. Other inhibitors include NCGC00185682, NCGC00183434 and NCGC00183681.

In other embodiments, the caspase inhibitors comprise the structures described in WO2017/079566, specifically, paragraph [0020] including IDN6556 (emricasan), IDN7314, IDN6734, CTS5814, IDN7568, CTS2891, CTS2357, CTS5674, CTS7186, CTS8931, QVD-OPh, VX166, IDN8126, IDN9103 and VX765 (belnacasn). Each of the chemical structures of the foregoing are hereby incorporated herein.

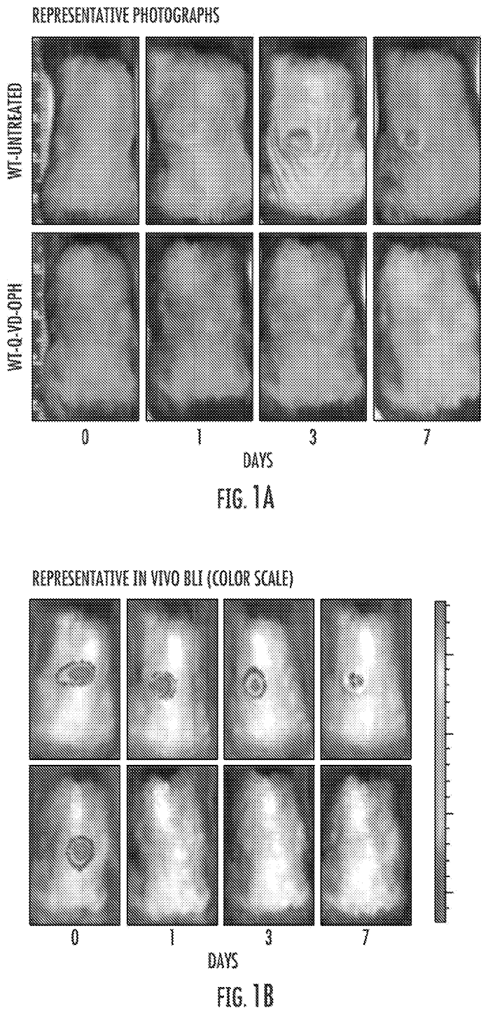

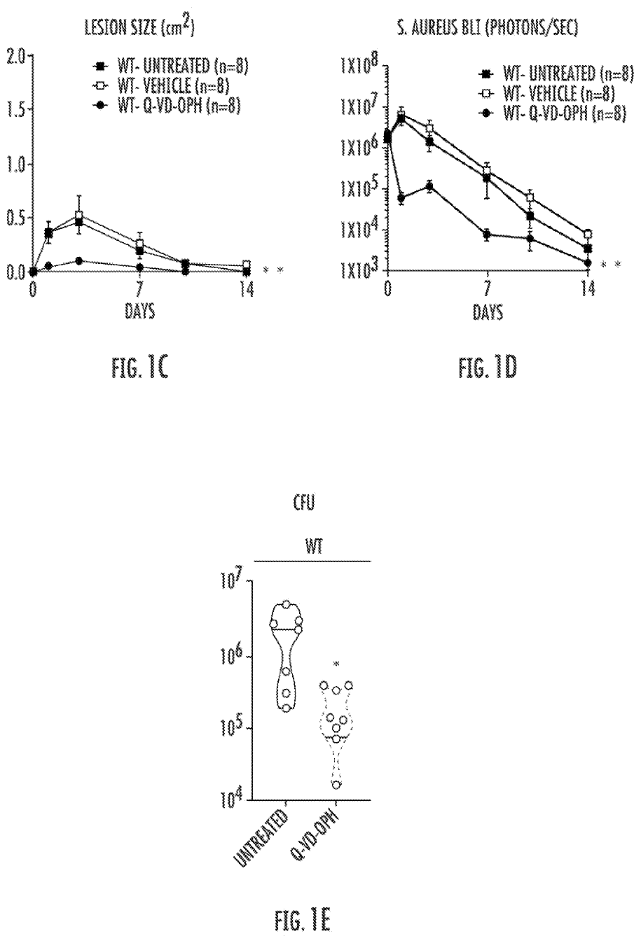

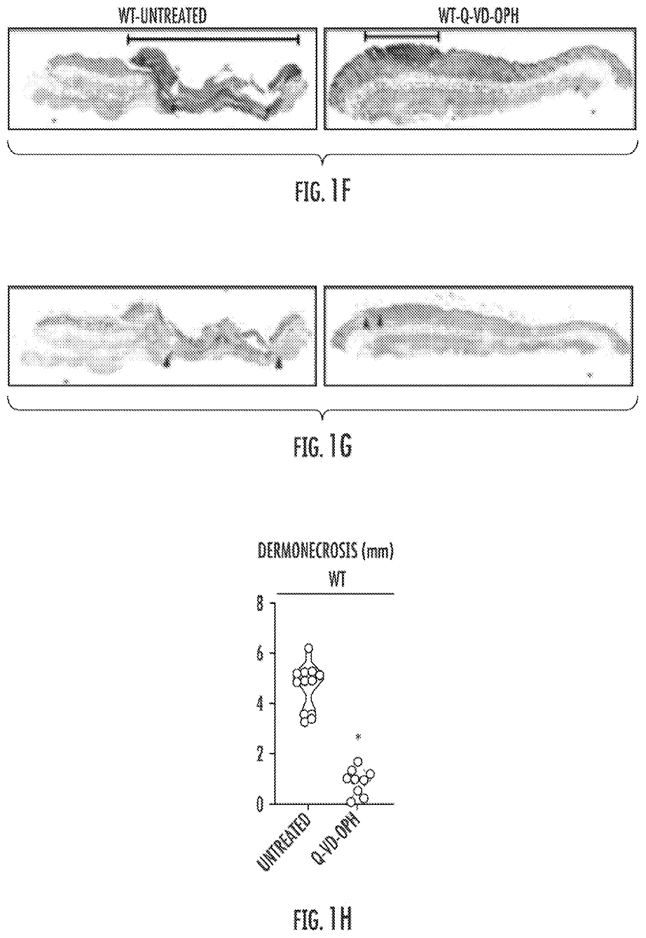

Representative compounds suitable for use with the present invention are disclosed in WO2012/134822. In particular, the compounds are disclosed in claims 1-11 and specific embodiments include NSC321205, NSC277584, NSC321206, and NSC310547 (see compounds A-D, respectively, in FIG. 1 and page 30, line 4). Each of the foregoing structures are hereby incorporated by reference herein.

Other caspase inhibitors include, but are not limited to, AC-YVAD-FMK; the caspase 3 and caspase 7 inhibitor AC-DEVD-CHO (N-acetyl-L-α-aspartyl-L-α-glutamyl-N-(2-carboxyl-1-formylethyl)-L-valinamide, 2,2,2-trifluoroacetate); the caspase-9 inhibitor Z-LEHD-FMK; the caspase-8 inhibitors Z-IETD-FMK (FMK007) and Emricasan (IDN-6556); the caspase-6 inhibitor Z-VEID-FMK; the caspase-3-like inhibitors Z-DEVD-CMK, MX1122 and M867; the caspase-3/7 selective inhibitor MMPSI and Isatin sulfonamides. Further examples include the broad-spectrum tripeptide inhibitors Boc-Asp-FMK, VX-166, Z-VAD-FMK (benzyloxycarbonyl-Val-Ala-Asp-fluoromethylketone), M867, Z-DEVD-CMK (benzyloxycarbonyl-Asp-Glu-Val-Asp-chloromethylketone), Ac-YVAD-CMK (acetyl-Tyr-Val-Ala-Asp-chloromethylketone), Z-LEHD-FMK (benzyloxycarbonyl-Leu-Glu-His-Asp-fluoromethylketone). Other inhibitors include VX-166, MX1122, YVAD-CHO, DEVD-CHO, MMPSI, M826, and Ac-DMQD-CHO.

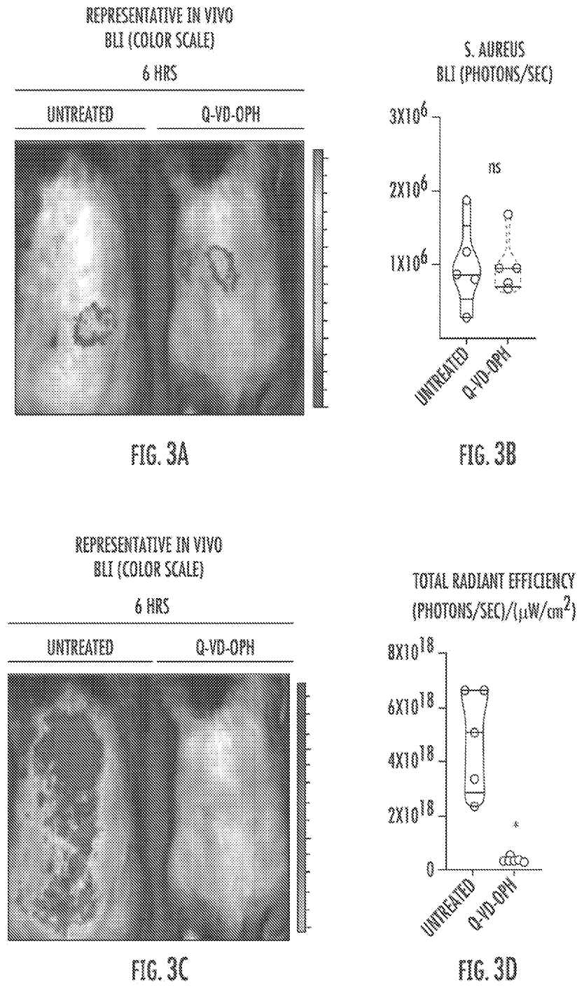

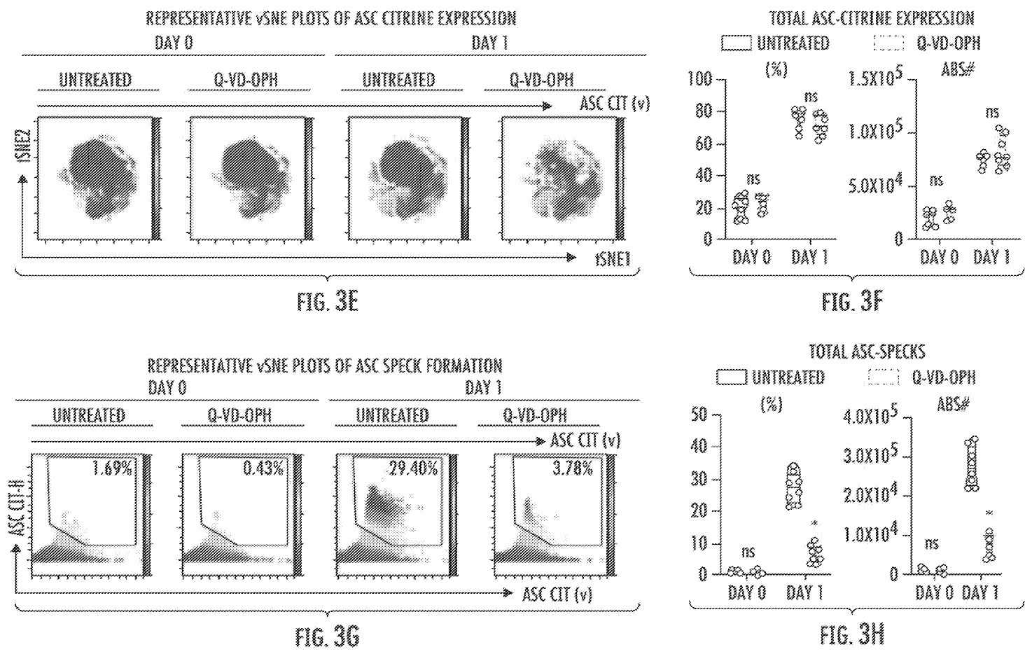

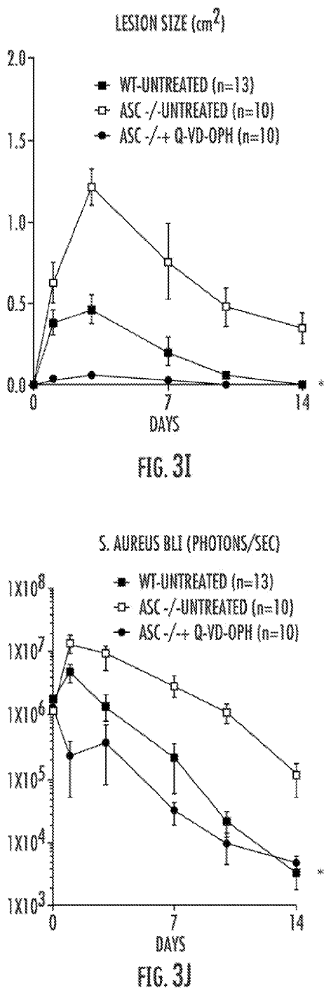

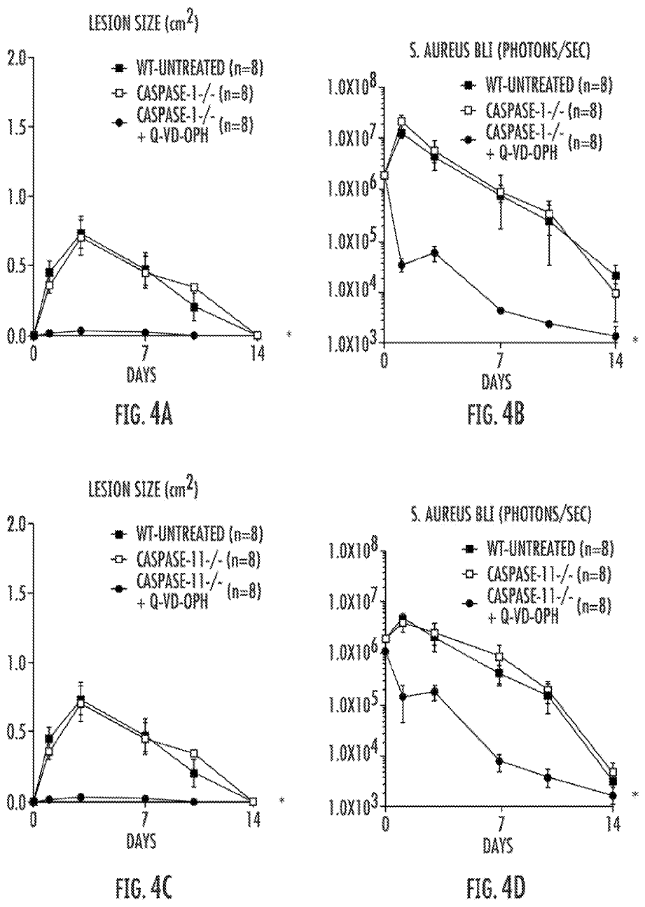

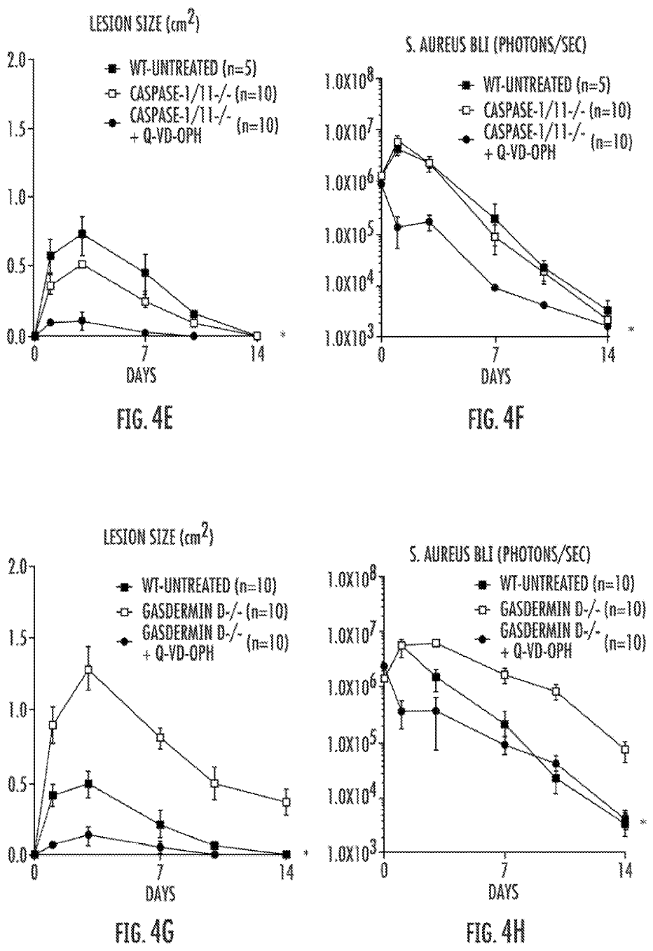

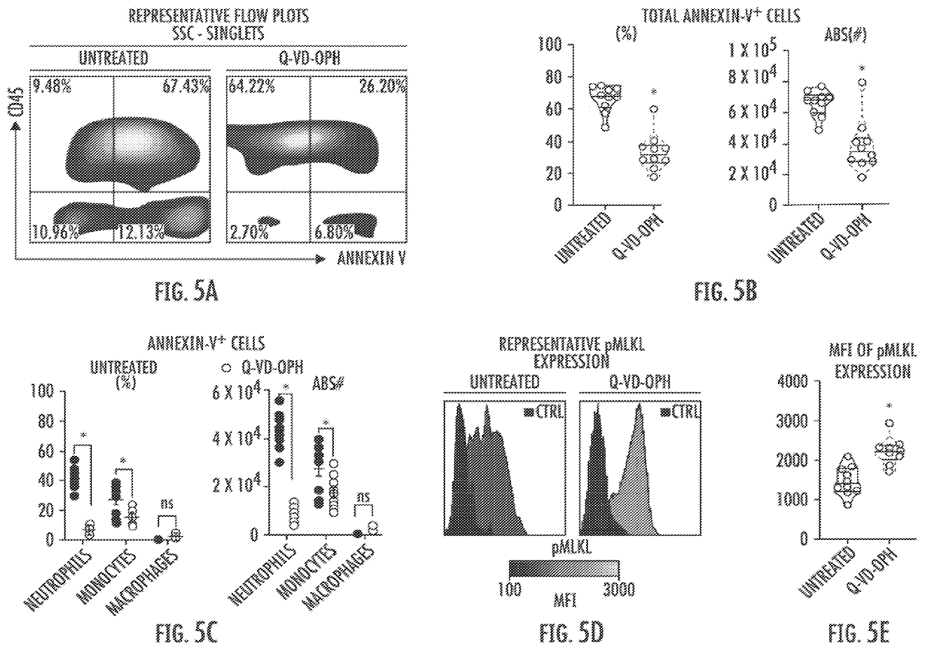

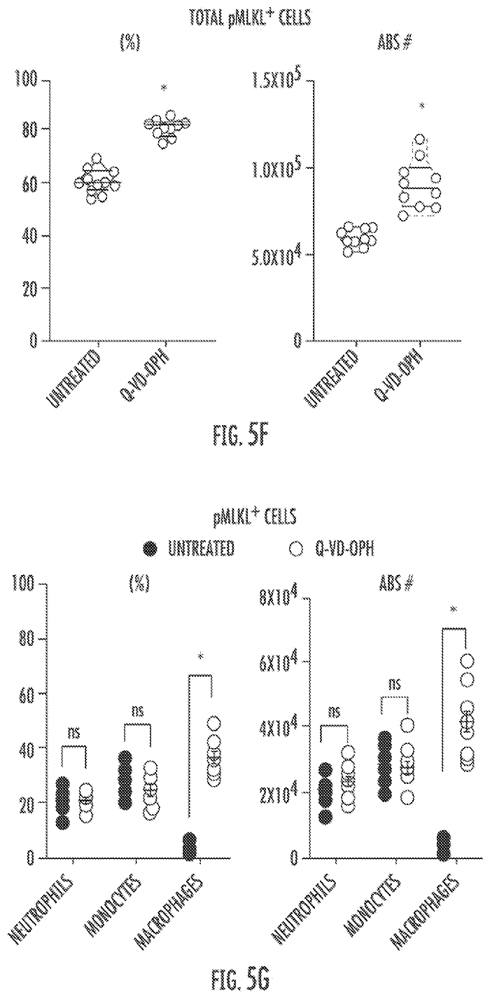

Representative compounds suitable for use with the present invention are disclosed in Lee et al., 28(1) EXPERT OPIN. THER. PATENTS 47-59 and include MJL-003i (FIG. 3(a)), MJL-002i (FIG. 3(b)), and MJL-001i (FIG. 3(c)). Other compounds in Lee et al. include the chemical structures of 2-[(4-formyl-pyrazol-5-yl)-thio]acetic acid derivatives (a) and 2-((1-((2,6-dimethylcyclohexachloride-1,5-dien-1-yl)methyl)-3-ethyl-4-formyl-1H-pyrazol yl)thio)acetic acid (b) (see FIG. 4). The chemical structures of SUN-6 (a), SUN-9 (b), SUN-19 (c) and SUN-21 (d), as shown in FIG. 5, are also representative.

The concentration of active compound (e.g., caspase inhibitor) in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of the conditions described herein.

In one embodiment, a therapeutically effective dosage should produce a serum concentration of an active ingredient of from about 0.1 ng/ml to about 50-100 μg/ml, from about 0.5 ng/ml to about 80 μg/ml, from about 1 ng/ml to about 60 μg/ml, from about 5 ng/ml to about 50 μg/ml, from about 5 ng/ml to about 40 μg/ml, from about 10 ng/ml to about 35 μg/ml, from about 10 ng/ml to about 25 μg/ml, from about 10 ng/ml to about 10 μg/ml, from about 25 ng/ml to about 10 μg/ml, from about 50 ng/ml to about 10 μg/ml, from about 50 ng/ml to about 5 μg/ml, from about 100 ng/ml to about 5 μg/ml, from about 200 ng/ml to about 5 μg/ml, from about 250 ng/ml to about 5 μg/ml, from about 500 ng/ml to about 5 μg/ml, from about 1 μg/ml to about 50 μg/ml, from about 0.1 ng/ml to about 5 ng/ml, from about 1 ng/ml to about 10 ng/ml or from about 1 μg/ml to about 10 μg/ml. The pharmaceutical compositions, in certain embodiments, should provide a dosage of from about 0.001 mg to about 2000 mg of compound per kilogram of body weight per day, from about 0.002 mg to about 1000 mg of compound per kilogram of body weight per day, from about 0.005 mg to about 500 mg of compound per kilogram of body weight per day, from about 0.005 mg to about 250 mg of compound per kilogram of body weight per day, from about 0.005 mg to about 200 mg of compound per kilogram of body weight per day, from about 0.005 mg to about 100 mg of compound per kilogram of body weight per day, from about 0.001 mg to about 0.005 mg of compound per kilogram of body weight per day, from about 0.01 mg to about 100 mg of compound per kilogram of body weight per day, from about 0.02 mg to about 100 mg of compound per kilogram of body weight per day, from about 0.05 mg to about 100 mg of compound per kilogram of body weight per day, from about 0.1 mg to about 100 mg of compound per kilogram of body weight per day, from about 0.5 mg to about 100 mg of compound per kilogram of body weight per day, from about 0.75 mg to about 100 mg of compound per kilogram of body weight per day, from about 1 mg to about 100 mg of compound per kilogram of body weight per day, from about 1 mg to about 10 mg of compound per kilogram of body weight per day, from about 0.001 mg to about 5 mg of compound per kilogram of body weight per day, from about 200 mg to about 2000 mg of compound per kilogram of body weight per day, or from about 10 mg to about 100 mg of compound per kilogram of body weight per day. Pharmaceutical dosage unit forms are prepared to provide from about 1 mg to about 1000 mg, from about 1 mg to about 800 mg, from about 5 mg to about 800 mg, from about 1 mg to about 100 mg, from about 1 mg to about 50 mg, from about 5 mg to about 100 mg, from about 10 mg to about 50 mg, from about 10 mg to about 100 mg, from about 25 mg to about 50 mg and from about 10 mg to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the condition being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable derivatives thereof are mixed with a suitable pharmaceutical carrier or vehicle for systemic, topical or local administration to form pharmaceutical compositions. Compounds are included in an amount effective for ameliorating one or more symptoms of, or for treating a condition described herein. The concentration of active compound in the composition will depend on absorption, inactivation, excretion rates of the active compound, the dosage schedule, amount administered, particular formulation as well as other factors known to those of skill in the art.

The compositions are intended to be administered by a suitable route, including orally, parenterally, rectally, topically, locally and via nasogastric or orogastric tube. For oral administration, capsules and tablets can be used. The compositions are in liquid, semi-liquid or solid form and are formulated in a manner suitable for each route of administration. In one embodiment, modes of administration include parenteral and oral modes of administration. In certain embodiments, oral administration is contemplated. In other embodiments, a topical administration is contemplated.

The caspase inhibitors of the present invention can be used in combination therapy to treat bacterial infections and skins lesions, viral infections, and to enhance regeneration and repair after skin or gut injury. As used herein, the term “in combination” refers to the use of more than one therapies (e.g., a caspase inhibitor and other agents (e.g., a TLR3 agonist)). The use of the term “in combination” does not restrict the order in which therapies (e.g., a caspase inhibitor and other agents) are administered to a subject with a disorder. In certain embodiments, a first therapy (e.g., a caspase inhibitor) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of another therapy (e.g., another caspase inhibitor or other agent) to a subject with a condition.

In various embodiments, the combinations can be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In certain embodiments, two or more therapies are administered within the same patient visit.

As used herein, the term “synergistic” refers to a combination of a caspase inhibitor with another caspase inhibitor or other agent such as a TLR3 agonist, which is more effective than the additive effects of the administration of the two compounds as monotherapies. A synergistic effect of a combination of therapies (e.g., a caspase inhibitor and another agent such as a TLR3 agonist) permits the use of lower dosages of one or more of the therapies and/or less frequent administration of the therapies to a subject with a disorder. The ability to utilize lower dosages of a therapy (e.g., a caspase inhibitor and another agent) and/or to administer the therapy less frequently reduces the toxicity associated with the administration of the therapy to a subject without reducing the efficacy of the therapy in the prevention or treatment of a disorder. In addition, a synergistic effect can result in improved efficacy of agents in the prevention or treatment of a disorder. Finally, a synergistic effect of a combination of therapies (e.g., a caspase inhibitor and another agent) may avoid or reduce adverse or unwanted side effects associated with the use of either therapy alone.

### III. Caspase Inhibitors in Combination with DS-RNA TLR3 Agonist(s)

In certain embodiments, caspase inhibitors can be administered in combination with double-stranded ribonucleic acid (DS-RNA) toll-like receptor 3 (TLR3 or Tlr3) (DS-RNA TLR3) agonist(s). In particular embodiments, the combination therapy can be used for regeneration and repair after skin or gut injury, scarring, wounds or any type of temporary injury to the skin or gut, as well as recovery after major surgery to the gut, after skin surgery or in case of burns.

### A. Definitions

The term “TLR3 agonist” refers to an affinity agent (e.g., a molecule that binds a target molecule) capable of activating a TLR3 polypeptide to induce a full or partial receptor-mediated response. An agonist of TLR3 may induce any TLR3 activity, for example TLR3-mediated signaling, either directly or indirectly. A TLR3 agonist, as used herein, may but is not required to bind a TLR3 polypeptide, and may or may not interact directly with the TLR3 polypeptide. A TLR agonist can also be a small molecule. Examples of TLR3 agonists/enhancers include, but are not limited to, dequalinium dicholoride, ivermectin, entandrophragmin, GW9662, P1,P4-Di(adenosine-5′)tetraphosphate triammonium, and astaxanthin.

As employed herein, the phrases “selective TLR3 agonist” and “TLR3 agonist which selectively induces TLR3 activity” refer to compositions which induce TLR3-mediated signalling to a significantly greater extent than signalling by one or more other dsRNA receptors. When the TLR3 agonist is a dsRNA composition, a “TLR3 agonist which selectively induces TLR3 activity” refers to compositions which induce TLR3-mediated signalling to a significantly greater extent than signalling by one or more other dsRNA receptors (e.g., TLR7, RIGI, MDA-5, PKR and/or other dsRNA receptors). In one embodiment, “significantly greater extent,” as applied to interaction between TLR3 agonist and a receptor, refers to agonists which have a significantly higher therapeutic index (i.e., the ratio of efficacy to toxicity) for treatment of the target disease state or condition than for activation of pathways mediated by other receptors. The toxicity of therapeutic compounds frequently arises from the non-selective interaction of the therapeutic compound with other receptors. Thus, the present invention provides a means to reduce the incidence of side-reactions commonly associated dsRNA therapy. Preferably, a composition which induces TLR3-mediated signalling to a significantly greater extent than signalling by other another receptor(s) will have an EC50 for induction of TLR3 signalling that is less than the EC50 for signalling by the other receptor(s).

“PolyI”, “polyC”, “polyA”, “polyU”, mean polyinosinic acid, polycytidylic acid, polyadenylic acid, and polyuridylic acid, respectively, each optionally substituted with other monomers.

“PolyAU”, used interchangeably with “pApU”, “polyA:U”, poly(A):poly(U), means an at least partially double stranded molecule made of polyadenylic acid(s) and polyuridylic acid(s), each optionally substituted with other monomers so long as the biological function (e.g., immunomodulatory activity, TLR3 agonism or binding) is preserved.

A “homopolymer” is a polymer made of substantially only a single monomer; for example a polyA homopolymer is substantially all A (adenosine) monomers. A homopolymer can be a single longer polymer or can consist of a plurality of shorter polymers concatenated (e.g., using a linker) to form a longer polymer, etc.

A “copolymer” is a polymer made of two or more monomers; for example a poly A copolymer comprises A (adenosine) monomers and one or more monomers other than adenosine.

The term “poly AxU” mean copolymer of adenylic acid and uridylic acid where one uridylic acid is substituted for about every x adenylic acids, respectively. For example “poly C12U” is a copolymer of cytidylic acid and uridylic acid where one uridylic acid is substituted for about every 12 cytidylic acids, respectively.

“dsRNA” and “double-stranded RNA” refer to complexes of polyribonucleotides which are at least partly double stranded. dsRNA need not be double stranded over the length of the molecule, nor over the length of one or more of the single-strand nucleic acid polymers that form the dsRNA. According to the invention, “dsRNA” means double-stranded RNA and is RNA with two partially or completely complementary strands. The size of the strands may vary from 6 nucleotides to 10000, preferably 10 to 8000, in particular 200 to 5000, 200 to 2000 or 200 to 1000 nucleotides. In certain embodiments, the dsRNA is polyinosinic-polycytidylic acid (poly(l:C)), a synthetic analog of dsRNA. Poly(l:C) is composed of a strand of poly(l) annealed to a strand of poly(C). The dsRNA can be a fully or partially (interrupted) pair of RNA hybridized together. It can be made for example by mixing polyinosinic and polycytidylic acid RNA molecules. It also can be made by mixing defined fully or partially pairing non-homopolymeric RNA strands. There is no specific ribonucleotide sequence requirement for the dsRNA molecules to be suitable for preparing a composition of the present invention.

The term “base pair” (abbreviated as “bp”) frequently used to indicate the molecular size of nucleic acid is used to indicate the molecular size by the numbers of bases in the nucleic acid (i.e., 10 bp means the double strand polymer having ten bases) in each complementary strand.

The term “biological sample” as used herein includes but is not limited to a biological fluid (for example serum, lymph, blood), cell sample or tissue sample (for example bone marrow).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “human-suitable” when referring to an agent or composition refers to any agent or composition that can be safely used in humans for, e.g., the therapeutic methods described herein. For example human suitable agents do not cause effects such as severe cytokine induction at a level that would preclude their use in humans, or contain levels of substances (e.g., endotoxins) that are incompatible with use in humans, in the particular context (e.g., mode of administration) in which the agent is used.

An “isolated” or “purified” preparation (e.g., dsRNA preparation) is substantially free of material or other contaminating compounds from the source from which the preparation (e.g., dsRNA) is derived, or substantially free from chemical precursors or other chemicals when chemically synthesized. “Substantially free” means that a preparation of dsRNA is at least 50% pure (wt/wt). In a preferred embodiment, the preparation of dsRNA has less than about 20%, 10%, 5% and more preferably 2% (by dry weight), of free ribonucleotide monomers, proteins or chemical precursors and/or other chemicals, endotoxins, and/or free ssRNA (in the case of a dsRNA preparation), e.g., from manufacture. These also referred to herein as “contaminants”. Examples of contaminants that can be present in a dsRNA preparation provided herein include, but are not limited to, calcium, sodium, ribonucleotide monomers, free ssRNA (in the case of a dsRNA preparation), endotoxin, polynucleotide phosphorylase enzyme (or other enzyme having similar substrate specificity), methanol, ethanol, chloride, sulfate, dermatan sulfate, and chondroitin sulfate. Purity and homogeneity are typically determined using analytical chemistry techniques such as polyacrylamide gel electrophoresis or high performance liquid chromatography.

The term “cathelicidins” refers to cationic peptides that have broad-range antimicrobial activity. Zanetti, M. et al. J. Biol. Chem. 268, 522 (1993). These peptides belong to the family of anti-microbial peptides which form part of the host's important innate immunity mechanism. Lehrer, R. and T. Ganz. Curr. Opin. Immunol. 11, 23 (1999). In humans, cathelicidins and defensins are expressed in immune cells and at epithelial surfaces. See Chromek, M. et al. Nature Medicine 12, 636 (2006); Zanetti, M. J. Leukoc. Biol. 75, 39 (2004); and Ganz, T. Nat. Rev. Immunol. 3, 710 (2003). hCAP18, human cationic antimicrobial protein, with a MW of 18 kD, is the only cathelicidin gene found in humans. Lehrer, R. and T. Ganz. Curr. Opin. Immunol. 11, 23 (1999). The N-terminus of this protein consists of a cathelin-like region (similar to the other members of the cathelicidin family) and a C-terminal termed LL-37. See Sorensen, O E. et al. Blood 97, 3951 (2001); and Zanetti, M. et al. FEBS Lett. 374, 1 (1995). An amphipathic alpha-helical peptide, LL-37 plays an important role in the first line of defense against local infection and systemic invasion of pathogens at sites of inflammation and wounds. Cytotoxic to both bacterial and normal eukaryotic cells, LL-37 is significantly resistant to proteolytic degradation in solution. See Neville, F. et al. Biophys. J. 90, 1275 (2006); and Oren, Z., et al. Biochem. J. 341, 501(1999).

Examples of cathelicidins include LL-37/hCAP18 (LL-37) in humans (Curr Drug Targets Inflamm Allergy. 2003 September; 2(3):224-31; Eur J. Biochem. 1996 Jun. 1; 238(2):325-32; Paulsen F et al., J. Pathol. 2002 November; 198(3):369-77). LL-37 is a 37 amino acid residue peptide corresponding to amino acid residue coordinates 134-170 of its precursor hCAP18/human cathelicidin antimicrobial peptide protein (GenBank: Accession NP004336; version NP004336.2 GI:39753970; REFSEQ: accession NM004345.3). LL-37 comprises the amino acid sequence LLGDFFRKSKEKIGKEFKRIVQRIKDFLRN LVPRTES (SEQ ID NO:1). The term LL-37 also includes sequences having at least 90% identity with SEQ ID NO:1. In particular, the term includes sequences having one or more conservative amino acid substitutions of SEQ ID NO:1. Cathelcidins including LL-37 can be used in the methods and composition described herein alone or in combination with dsRNA or other TLR3 agonists to enhance hair follicle neogenesis and/or regeneration.

### B. DS-RNA TLR3 Agonist(s)

Double-stranded (ds) RNA (ribonucleic acid) is chemically very similar to DNA (deoxyribonucleic acid). It is also a long molecule containing nucleotides linked together by 3′-5′ phosphodiester bonds. Two differences in its chemical groups distinguish dsRNA from DNA. The first is a minor modification of sugar component. The sugar of DNA is deoxyribose, whereas RNA contains ribose, which is identical to deoxyribose except for the presences of an additional hydroxyl group. The second difference is that RNA contains no thymine, but instead contains the closely related pyrimidine, uracil. DsRNA forms from the hybridization of two complementary polyribonucleotides forming a double helix similar to that of DNA. The two strands of the double helix are held together by hydrogen-bonded base pairs.

TLR3 is a receptor for a form of immunity called “innate immunity” which recognizes double-stranded RNAs with a minimum size of at least 50 base pairs. The size requirement or discrimination of dsRNA by TLR3 prevents responses to non-microbial sources of dsRNA micro (mi) RNA or transfer (t) RNA. TLR3 exists as a horseshoe shaped monomer with an N-terminal, ligand-binding extra-cytoplasmic domain (ECD), a transmembrane domain (TMD), and a C-terminal cytoplasmic signaling domain (CSD). X-ray crystallographic studies have provided structural data for the TLR-3 ligand complex which consists of a TLR3 homo-dimer complexed to dsRNA of at least about 50 consecutive base pairs. The formation of the complex is believed to transmit a conformational change in the CSD via the TMD connector that allows cytoplasmic signaling. Above 50 base pairs, binding affinity is a function of size with a progressive increase in binding affinity with increased length in linear non-branched dsRNA. The minimum size for dsRNA is about 40 nucleotides.

The double-stranded ribonucleic acid (dsRNA) may be fully hybridized strands of poly(riboinosinic acid) and poly(ribocytidilic acid) (i.e., polyIC) or poly(riboadenylic acid) and poly(ribouracilic acid) (i.e., polyAU). If mismatched, the dsRNA may be of the general formula rIₙ·r(C₄₋₂₉U)ₙ, which is preferably rIₙ·r(C₁₂U)ₙ, in which r indicates ribonucleotides. It is preferred that n is an integer from about 40 to about 40,000. For example, a strand of poly(riboinosinic acid) may be partially hybridized to a strand of poly(ribocytosinic₄₋₂₉uracilic acid). Other mismatched dsRNA that may be used are based on copolynucleotides such as poly(CₘU) and poly(CₘG) in which m is an integer from about 4 to about 29 or analogs of a complex of poly(riboinosinic acid) and poly(ribocytidilic acid) formed by modifying the rIₙ·rCₙ to incorporate unpaired bases (uracil or guanine) in the polyribocytidylate (rCₘ) strand. Alternatively, mismatched dsRNA may be derived from r(I)r(C) dsRNA by modifying the ribosyl backbone of poly(riboinosinic acid) (rIₙ), e.g., by including 2′-O-methyl ribosyl residues. Of these mismatched dsRNA analogs of rIₙ rCₙ, the preferred ones are of the general formula rIₙ·r(C₁₁₋₁₄U)ₙ or rIₙ·r(C₂₉,G)ₙ (see U.S. Pat. Nos. 4,024,222 and 4,130,641; which are incorporated by reference). The dsRNA described therein generally are suitable for use according to the present invention. See also U.S. Pat. No. 5,258,369.

The dsRNA may be complexed with an RNA-stabilizing polymer such as polylysine, polylysine plus carboxy-methylcellulose, polyarginine, polyarginine plus carboxymethylcellulose, or any combination thereof. Other examples of mismatched dsRNA for use in the invention include, but are not limited to, r(I)·r(C₄,U); r(I)·r(C₇,U); r(I)·r(C₁₃,U); r(I)·r(C₂₂,U); r(I)·r(C₂₀,G); and r(I)·r(C₂₉,G). Mismatched dsRNA may also be modified at the molecule's ends to add a hinge(s) to prevent slippage of the base pairs, thereby conferring a specific bioactivity in specific solvents or aqueous environments which exist in human biological fluids.

Poly-ICLC (interchangeably known as Hiltonol® or poly-IC:LC, among others) is a high molecular weight derivative of poly-IC stabilized with poly L-lysine and carboxymethylcellulose (CMC) that have been added to improve the pharmacokinetic properties of poly-IC. Poly-ICLC therefore has a formula of ln·Cn-poly-1-lysine-5 carboxymethylcellulose. See U.S. Pat. No. 4,349,538. Carboxymethylcellulose is a negatively charged (at neutral pH), hydrophilic material used to maintain the solubility of the complex. PolyICLC is more resistant to nucleases than poly-IC with a 27,000 KDa or larger complex of poly-ICLC being particularly resistant to nucleases.

In specific embodiments, the dsRNA TLR3 agonist is Ampligen®. Ampligen® is a particular dsRNA denoted Poly I:Poly C₁₂U, wherein one of the two polyribonucleotides is polyriboinosinic acid and the other is polyribocytidylic₁₂, uridylic acid. Thus, the pyrimidine building blocks of Ampligen® are present in a ratio of 12 cytosines of each uracil, while the complementary purine strand contains 13 inosine residues. Within the double-stranded helical structure of Ampligen® the pyrimidine, cytosine, hydrogen bonds with the purine, inosine, while the pyrimidine, uracil, does not form any hydrogen bonds. Therefore, a “mismatch” is created once for every 12 base pairs (bps) formed between the inosine and cytosine residues. In contrast to Ampligen®, Poly I:Poly C contains only complementary inosine: cytosine base pairs. No uracil is present in Poly I:Poly C and there are no mismatches.

Other agonists of TLR3 that may be useful in embodiments of the invention include Poly-ICR (Poly IC (Polyriboinosinic-polycytidylic acid)-Poly arginine (Nventa Biopharmaceuticals Corporation); high MW synthetic dsRNA IPH31XX compounds, for example IPH3102, which in humans are specific for TLR3 (Innate Pharma S.A; Schering-Plough Corporation); Oragens™, for example Oragen™ 0004, Oragen™ 0033 and Oragen™ 0044 (Temple University); and NS9, a complex of polyinosinic-polycytidylic acid (Nippon Shinyaku Co., Ltd). The Oragen™ compounds are synthetic analogues of naturally occurring 2′,5′-oligoadenylate analogues, wherein the analogues are typically conjugated to a carrier molecule to enhance cellular uptake (see U.S. Pat. No. 6,362,171).

PCT Publication No. WO 2009/130616 (Innate Pharma) describes high MW polyAU dsRNA molecules that are TLR3 agonists. PCT Publication Nos. WO 2006/054177, WO 2006/054129, WO 2009/130301 and WO 2009/136282 (Institut Gustave Roussy) describe the use of dsRNA TLR3 agonists for treating cancer.

Further embodiments are also disclosed in WO 2007/089151, which describes stathmin and stathmin-like compounds that are TLR3 agonists. In a specific embodiment, a nucleic acid-based agonist is coupled to one of these stathmin or stathmin-like agonists.

In another embodiment, the dsRNA TLR4 agonist is rugged dsRNA. Rugged dsRNA is a novel form of dsRNA with a unique composition and physical characteristics. Unlike the previously known antiviral, Ampligen® (Poly I:Poly C₁₂U), the new and improved form of Rugged dsRNA (e.g., Poly I:Poly C₃₀₋₃₅U (preferably, Poly I:Poly C₃₀U), wherein PolyC₃₀₋₃₅U, indicates a ratio, that is, that for every U there are 30-35 C's), has an increased Ruggedness characterized by an increase resistance to thermal denaturation and ribonuclease digestion. This improved form of dsRNA also has a reduced tendency to form branched dsRNA molecules which results in increased bioactivity due to an increased ability to bind TLR3 receptor. The minimal length of Rugged dsRNA (termed the monomer unit) is about 50 base pairs requiring about 4 to 5 (e.g., 4.7) helical turns (10.7 base pairs are required for each complete turn of the helix) within its dsRNA structure and represents the smallest or monomeric unit of Poly I:Poly C₃₀U, approximately 24,000 to 30,000 Daltons (a Dalton is a unit of weight equal to the weight of a single hydrogen atom). The maximal length of Rugged dsRNA is about 500 base pairs composed of about 10 monomer units, requiring about 50 (e.g., 46.7) helical turns and having a molecular weight of approximately 300,000 Daltons (e.g., about 225,000 Daltons). See U.S. Patent Application Publication No. 20120009206.

### C. Formulations and Pharmaceutical Compositions Comprising a TLR Agonist

In a preferred embodiment, the compositions comprising a TLR3 agonist are administered topically. It is preferable to present the active ingredient, i.e. TLR3 agonist as a pharmaceutical formulation. Exemplary compositions are described in detail in the examples which follow. The active ingredient may comprise, for topical administration, from 0.001% to about 20% w/w, by weight of the formulation in the final product, although it may comprise as much as 30% w/w, from about 1% to about 20% w/w of the formulation. The topical formulations of the present invention, comprise an active ingredient together with one or more acceptable carrier(s) therefor and optionally any other therapeutic ingredients(s). The carriers must be “acceptable” in the sense of being compatible with the other ingredients of the formulation and not deleterious to the recipient thereof.

The TLR3 agonist composition of the present invention can be administered to a patient either by itself or in pharmaceutical compositions where it is mixed with suitable carriers or excipient(s). In treating a patient exhibiting a disorder of interest, a therapeutically effective amount of an agent or agents such as these is administered. A therapeutically effective dose refers to that amount of the compound that results in amelioration of symptoms or a prolongation of survival in a patient.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, capsules, or solutions. The pharmaceutical compositions of the present invention may be manufactured in a manner that is itself known, e.g., by means of conventional mixing, dissolving, granulating, emulsifying, encapsulating, entrapping or lyophilizing processes.

The compositions described above may be administered to a subject in any suitable formulation. In addition to treatment with topical formulations of TLR3 agonist, in other aspects of the invention TLR3 agonist might be delivered by other methods. For example, TLR3 agonist might be formulated for parenteral delivery, e.g., for subcutaneous, intravenous, or intramuscular injection. Other methods of delivery, for example, liposomal delivery or diffusion from a device impregnated with the composition might be used. The compositions may be administered in a single bolus, multiple injections, or by continuous infusion (for example, intravenously or by peritoneal dialysis). For parenteral administration, the compositions are preferably formulated in a sterilized pyrogen-free form.

Formulations suitable for topical administration include liquid or semi-liquid preparations suitable for penetration through the skin to the site of where treatment is required, such as liniments, lotions, creams, ointments or pastes. Lotions according to the present invention include those suitable for application to the skin. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active such as sorbitan esters or polyoxyethylene derivatives thereof. Suspending agents such as natural gums, cellulose derivatives or inorganic materials such as siliceous silicas, and other ingredients such as lanolin, may also be included.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxy-methylcellulose, and/or polyvinyl pyrrolidone (PVP). If desired, disintegrating agents may be added, such as the cross-linked polyvinyl pyrrolidone, agar, or alginic acid or a salt thereof such as sodium alginate.

For preferred topical delivery vehicles the remaining component of the composition can be water, which is necessarily purified, e.g., deionized water. Such delivery vehicle compositions can contain water in the range of more than about 50 to about 95 percent, based on the total weight of the composition. The specific amount of water present is not critical, however, being adjustable to obtain the desired viscosity (usually about 50 cps to about 10,000 cps) and/or concentration of the other components.

Other known transdermal skin penetration enhancers can also be used to facilitate delivery of a TLR3 agonist. Illustrative are sulfoxides such as dimethylsulfoxide (DMSO) and the like; cyclic amides such as 1-dodecylazacycloheptane-2-one (Azone™, a registered trademark of Nelson Research, Inc.) and the like; amides such as N,N-dimethyl acetamide (DMA) N,N-diethyl toluamide, N,N-dimethyl formamide, N,N-dimethyl octamide, N,N-dimethyl decamide, and the like; pyrrolidone derivatives such as N-methyl-2-pyrrolidone, 2-pyrrolidone, 2-pyrrolidone-5-carboxylic acid, N-(2-hydroxyethyl)-2-pyrrolidone or fatty acid esters thereof, 1-lauryl-4-methoxycarbonyl-2-pyrrolidone, N-tallowalkylpyrrolidones, and the like; polyols such as propylene glycol, ethylene glycol, polyethylene glycol, dipropylene glycol, glycerol, hexanetriol, and the like; linear and branched fatty acids such as oleic, linoleic, lauric, valeric, heptanoic, caproic, myristic, isovaleric, neopentanoic, trimethyl hexanoic, isostearic, and the like; alcohols such as ethanol, propanol, butanol, octanol, oleyl, stearyl, linoleyl, and the like; anionic surfactants such as sodium laurate, sodium lauryl sulfate, and the like; cationic surfactants such as benzalkonium chloride, dodecyltrimethylammonium chloride, cetyltrimethylammonium bromide, and the like; non-ionic surfactants such as the propoxylated polyoxyethylene ethers, e.g., Poloxamer 231, Poloxamer 182, Poloxamer 184, and the like, the ethoxylated fatty acids, e.g., Tween 20, Myrj 45, and the like, the sorbitan derivatives, e.g., Tween 40, Tween 60, Tween 80, Span 60, and the like, the ethoxylated alcohols, e.g., polyoxyethylene (4) lauryl ether (Brij 30), polyoxyethylene (2) oleyl ether (Brij 93), and the like, lecithin and lecithin derivatives, and the like; the terpenes such as D-limonene, α-pinene, β-carene, α-terpineol, carvol, carvone, menthone, limonene oxide, α-pinene oxide, eucalyptus oil, and the like. Also suitable as skin penetration enhancers are organic acids and esters such as salicyclic acid, methyl salicylate, citric acid, succinic acid, and the like.

One skilled in the art will appreciate that a suitable or appropriate formulation can be selected, adapted or developed based upon the particular application at hand. Dosages for presently disclosed compositions can be in unit dosage form. The term “unit dosage form” as used herein refers to physically discrete units suitable as unitary dosages for animal (e.g. human) subjects, each unit containing a predetermined quantity of a presently disclosed agent, alone or in combination with other therapeutic agents, calculated in an amount sufficient to produce the desired effect in association with a pharmaceutically acceptable diluent, carrier, or vehicle. Indeed, one skilled in the art can easily determine the appropriate dose, schedule, and method of administration for the exact formulation of the composition being used, in order to achieve the desired effective amount or effective concentration of the agent in the individual patient.

The dose of a presently disclosed composition, administered to an animal, particularly a human, in the context of the presently disclosed subject matter should be sufficient to produce at least a detectable amount of a therapeutic response in the individual (e.g., stimulate hair follicle neogenesis) over a reasonable time frame. The dose used to achieve a desired effect will be determined by a variety of factors, including the potency of the particular agent being administered (e.g., a TLR3 agonist), the pharmacodynamics associated with the agent in the host, the severity of the condition in the subject, other medications being administered to the subject, the degree of susceptibility of the individual, the age, sex, and weight of the individual, idiosyncratic responses of the individual, and the like. The size of the dose also will be determined by the existence of any adverse side effects that may accompany the particular agent, or composition thereof, employed. It is generally desirable, whenever possible, to keep adverse side effects to a minimum. The dose of the biologically active material will vary; suitable amounts for each particular agent will be evident to a skilled worker.

Accordingly, in certain embodiments, the compositions can be administered/applied at a dose of about 1-100 μg/cm² including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/cm² area per application.

In a more specific embodiment, the compositions can be administered/applied in a range of about 1-20 μg/cm² area per application including, but not limited to, 1-19, 1-18, 1-17, 1-16, 1-15, 1-14, 1-13, 1-12, 1-11, 1-10, 1-9, 1-8, 1-7, 1-6, 1-5, 1-4, 1-3, 1-2, 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, and 19-20 μg/cm² area per application.

The pharmaceutical compositions can be administered on a daily basis. In one embodiment, the compositions are administered once a day for 2, 3, 4, 5, 6, 7, 8, 9, 10, 11, 12, 13, 14, 15, 16, 17, 18, 19, 20, 21, 22, 23, 24, 25, 26, 27, 28, 29, 30 or more days including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 weeks or more including 1, 2, 3, 4, 5, 6, 7, 8, 9, 10, 11, or 12 months.

The compositions can be administered once every few days including once every 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. The compositions can be administered once a week for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or more. Alternatively, the compositions can be administered once every few weeks for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or more.

In other embodiments, the compositions can be administered several times in a month including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 times per month.

In particular embodiments, the dose of a composition described herein comprises a range of about 2-10 μg/cm² area per application, with 1-10 applications separated within one month. In other embodiments, the dosage is about 2-10 μg/cm² area per application, with 1-3 applications separated within one month.

### IV. Caspase Inhibitor to Treat Bacterial Infection and Skin Lesions

The present invention provides caspase inhibitors as an alternative to antibiotics by engaging the host immune response to promote clearance of bacterial infections. In the U.S., there are 11 million outpatient/ER visits and 500,000 hospital admissions per year. Also, there are 2 million people who suffer from invasive antibiotic-resistant infections with 23,000 deaths per year in the U.S. alone. The use of caspase inhibitors in the present invention is ground-breaking as it could combat the bacterial infection as an alternative or complement antibiotic therapy. This will improve patient outcomes and prevent the spread of antibiotic resistance. As described in Example 2 and the Figures, a caspase inhibitor was used to decrease bacterial burden and skin lesion sizes against a Staphylococcus aureus skin infection, a group A Streptococcus (Streptococcus pyogenes) skin infection and a Pseudomonas aeruginosa skin infection.

Examples of bacteria include, but are not limited, to staphylococcus (for example, Staphylococcus aureus, Staphylococcus epidermidis, or Staphylococcus saprophyticus), streptococcus (for example, Streptococcus pyogenes, Streptococcus pneumoniae, or Streptococcus agalactiae), enterococcus (for example, Enterococcus faecalis, or Enterococcus faecium), corynebacteria species (for example, Corynebacterium diptheriae), bacillus (for example, Bacillus anthracis), listeria (for example, Listeria monocytogenes), Clostridium species (for example, Clostridium perfringens, Clostridium tetanus, Clostridium botulinum, Clostridium difficile), Neisseria species (for example, Neisseria meningitidis, or Neisseria gonorrhoeae), E. coli, Shigella species, Salmonella species, Yersinia species (for example, Yersinia pestis, Yersinia pseudotuberculosis, or Yersinia enterocolitica), Vibrio cholerae, Campylobacter species (for example, Campylobacter jejuni or Campylobacter fetus), Helicobacter pylori, pseudomonas (for example, Pseudomonas aeruginosa or Pseudomonas mallei), Haemophilus influenzae, Bordetella pertussis, Mycoplasma pneumoniae, Ureaplasma urealyticum, Legionella pneumophila, Treponema pallidum, Leptospira interrogans, Borrelia burgdorferi, mycobacteria (for example, Mycobacterium tuberculosis), Mycobacterium leprae, Actinomyces species, Nocardia species, chlamydia (for example, Chlamydia psittaci, Chlamydia trachomatis, or Chlamydia pneumoniae), Rickettsia (for example, Rickettsia ricketsii, Rickettsia prowazekii or Rickettsia akari), brucella (for example, Brucella abortus, Brucella melitensis, or Brucella suis), Proteus mirabilis, Serratia marcescens, Enterobacter clocae, Acetinobacter anitratus, Klebsiella pneumoniae and Francisella tularensis. In specific embodiments, the bacteria is staphylococcus, streptococcus, enterococcus, bacillus, Clostridium species, E. coli, yersinia, pseudomonas, Proteus mirabilis, Serratia marcescens, Enterobacter clocae, Acetinobacter anitratus, Klebsiella pneumoniae or Mycobacterium leprae. In more specific embodiments, the bacteria is Staphylococcus aureus, Staphylococcus epidermidis, Streptococcus pyogenes, Pseudomonas aeruginosa, Enterococcus faecalis, Proteus mirabilis, Serratia marcescens, Enterobacter clocae, Acetinobacter anitratus, Klebsiella pneumoniae and/or Escherichia coli.

Without further elaboration, it is believed that one skilled in the art, using the preceding description, can utilize the present invention to the fullest extent. The following examples are illustrative only, and not limiting of the remainder of the disclosure in any way whatsoever.

### Examples

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

EXAMPLE 1: Pan-Caspase Inhibition as Host-Directed Immunotherapy Against MRSA and Other Bacterial Skin Infections. Staphylococcus aureus causes the majority of skin infections in humans and the emergence of methicillin-resistant S. aureus (MRSA) strains is a serious public health threat. There is an urgent clinical need for non-antibiotic immunotherapies to treat MRSA infections and prevent spread of antibiotic resistance. Herein, the pan-caspase inhibitor quinoline-val-asp-difluorophenoxymethyl ketone (Q-VD-OPH) was investigated for efficacy against a MRSA skin infection in mice. A single systemic dose of Q-VD-OPH rapidly decreased skin lesion sizes and cleared the bacteria, compared with vehicle or untreated wildtype (WT) mice. Although Q-VD-OPH inhibited inflammasome-dependent ASC speck formation and caspase-1-mediated IL-1β production, Q-VD-OPH maintained efficacy in mice deficient in IL-1β, ASC, caspase-1, caspase-11 or Gasdermin D. This unexpectedly indicated that the Q-VD-OPH efficacy was independent of inflammasome-mediated pyroptosis. Rather, Q-VD-OPH reduced apoptosis of monocytes and neutrophils. Moreover, Q-VD-OPH enhanced necroptosis of macrophages with concomitant increased serum TNF levels and TNF-producing neutrophils and monocytes/macrophages and neutrophils in the infected skin. Consistently, Q-VD-OPH lacked efficacy in mice deficient in TNF (with associated reduced neutrophil influx and necroptosis), mice deficient in TNF/IL-1R and in anti-TNF antibody-treated WT mice. The in vivo efficacy of Q-VD-OPH likely occurred through inhibition of multiple caspases as in vitro studies revealed that combined caspase 3, 8 and 9 inhibition reduced apoptosis and combined caspase 1, 8 and 11 inhibition increased TNF. Finally, Q-VD-OPH also had a therapeutic effect against Streptococcus pyogenes and Pseudomonas aeruginosa skin infections in mice. Collectively, pan-caspase inhibition represents a potential host-directed immunotherapy against MRSA and other bacterial skin infections.

### Materials and Methods

Caspase inhibitors. The pan-caspase inhibitor quinoline-val-asp-difluorophenoxymethyl ketone (Q-VD-OPH) (Cayman Chemicals) was dissolved in DMSO to create a 5 mg/mL stock solution. The in vivo dose of 20 mg/kg diluted in sterile PBS was administered intraperitoneally (i.p.) to mice. For in vitro culture studies, Q-VD-OPH (10, 100 and 10,000 μg/mL) as well as specific caspase inhibitors Z-WEHD-FMK (caspase-1 inhibitor) (100 μM) (R&D Systems), Z-DQMD-FMK (caspase-3 inhibitor) (10 μM) (R&D Systems), Z-IETD-FMK (caspase-8 inhibitor) (100 μM) (R&D Systems), Z-LEHD-FMK (caspase-9 inhibitor) (100 μM) (R&D Systems), wadelolactone (caspase-11 inhibitor) (20 μM) (Santa Cruz Biotechnology) and pan-caspase inhibitor Emricasan (Selleckchem) (9 μg/mL) were prepared according to the manufacturer instructions. There is no specific inhibitor available for caspase-7.

Bacterial strains. The bioluminescent S. aureus USA300 LAC::lux strain was previously generated from the community-acquired methicillin-resistant S. aureus (MRSA) USA300 LAC isolate obtained from a skin infection outbreak in the Los Angeles County Jail (Los Angeles, Calif., USA) and was kindly provided by Tammy Kielian (University of Nebraska). The bioluminescent S. pyogenes strain Xen20 (PerkinElmer, Hopkinton, Mass.) was derived from the parental S. pyogenes strain 591 serotype M49 strain. The bioluminescent P. aeruginosa strain Xen41 (PerkinElmer) was obtained from the parental strain PAO1. USA300 LAC::lux, Xen20 and Xen41 all possess a modified lux operon from Photorhabdus luminescens stably integrated into the bacterial chromosome so that the emission of blue-green light from live and metabolically active bacteria is maintained in all progeny without selection.

Bacterial preparation. S. aureus USA300 LAC::lux bacteria were streaked onto a tryptic soy agar (TSA) plate (tryptic soy broth [TSB] plus 1.5% bacto agar; BD Biosciences) and grown overnight at 37° C. in a bacterial incubator. Single colonies were cultured in TSB at 37° C. in a shaking incubator (240 rpm) overnight (˜18 hours), followed by a 1:50 subculture at 37° C. for 2 hours to obtain mid-logarithmic growth phase bacteria. S. pyogenes strain Xen20 was streaked on THY plates (Todd-Hewitt broth [Neogen] plus 0.5% yeast extract [MilliporeSigma] plus 1.5% bacto agar) and grown overnight in a bacterial incubator. Single colonies of Xen20 were grown overnight in THY broth (Todd-Hewitt broth [Neogen] plus 0.5% yeast extract [MilliporeSigma]) at 37° without shaking, followed by 1:25 dilution in THY broth and grown for 4 hours at 37° C. without shaking to obtain mid-logarithmic growth phase bacteria. P. aeruginosa strain Xen41 bacteria were streaked onto a Luria-Bertani (LB) plate (LB broth plus 1.5% bacto agar) and grown overnight in a bacterial incubator. Single colonies of P. aeruginosa strain were grown overnight in LB broth at 37° C. shaking at 240 rpm, then diluted 1:50 and grown for 2.5 hours to obtain mid-logarithmic growth phase bacteria. For USA300 LAC::lux, Xen41 and Xen20, each bacterial strain was separately pelleted, washed and resuspended in PBS and the absorbance at 600 nm (A600) was measured to estimate the number of CFU for the predetermined inoculum (USA300 LAC::lux [3×10⁷ CFU(25)], S. pyogenes Xen20 [5×10⁵ CFU] or P. aeruginosa Xen41 [5×10⁶ CFU] (43)) for each strain, which was verified after overnight culture on plates.

Mice. All mice were on a C57BL/6 background. C57BL/6 wildtype (WT) mice, ASC Citrine (B6.Cg-Gt(ROSA)26Sor^{tm1.1(CAG-Pycard/mCitrine*,-CD2*)Dtg}/J), Caspase-1/11^{-/-} (B6N.129S2-Casp1^{tm1Flv}/J), TNF^{-/-} (B6;129S-Tnf^{tm1Gkl}/J) and IL-1R^{-/-} (B6.129S7-illr1^{tmlmx}/J) mice were obtained from Jackson Laboratories (Bar Harbor, Me.). Mice deficient in both TNF and IL-1R (TNF/IL-1R^{-/-} mice) were generated by crossing the TNF^{-/-} with the IL-1R^{-/-} mice. IL-1β^{-/-} mice were previously generated and provided by Yoichiro Iwakura (University of Tokyo). ASC^{-/-}, GSDMD^{-/-} (Gasdermin D-deficient), caspase-1^{-/-} and caspase-11^{-/-} mice were previously generated and provided by Genentech (San Francisco, Calif.). All mice were bred and maintained under specific pathogen-free conditions at an animal facility accredited by the American Association for the Accreditation of Laboratory Animal Care (AAALAC) at Johns Hopkins and housed according to procedures described in the Guide for the Care and Use of Laboratory Animals (National Academies Press, 2011).

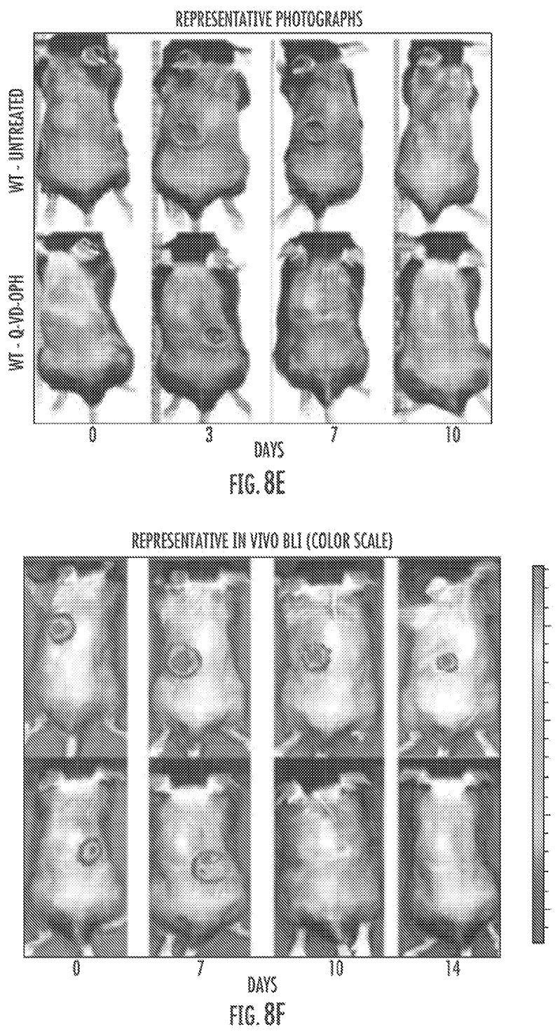

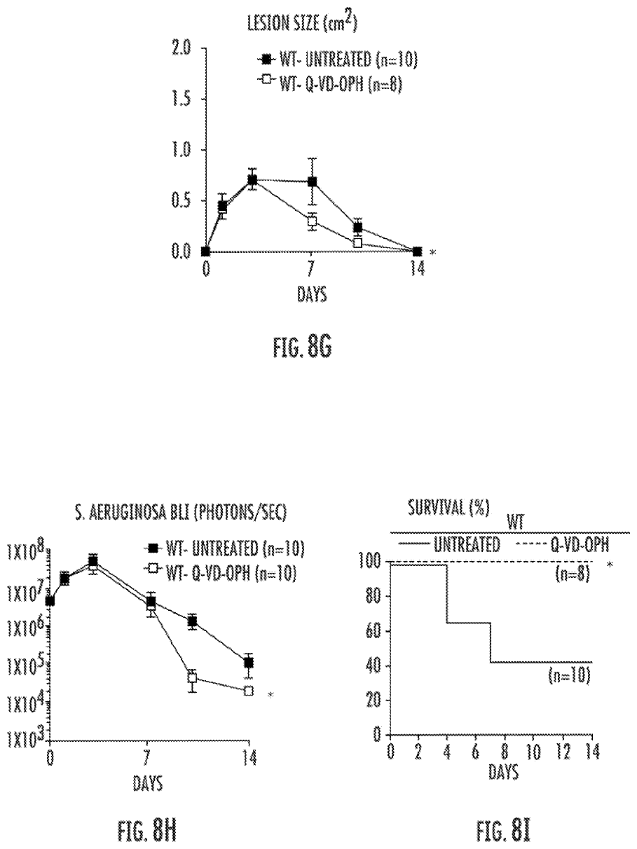

In vivo mouse models of bacterial skin infections. All animal studies were approved by the Johns Hopkins University Animal Care and Use Committee. For all experiments, 6-8-week-old sex- and age-matched mice were used. The dorsal backs of anesthetized (inhalation isoflurane [2%]) mice were shaved and inoculated via intradermal (i.d.) injection of CA-MRSA strain USA300 LAC::lux (3×10⁷ CFU (25, 26)), S. pyogenes strain Xen20 (5×10⁵ CFU (42)) or P. aeruginosa strain Xen41 (5×10⁶ CFU (43)) in 100 μL of PBS using a 29-gauge insulin syringe. In some experiments, an anti-TNF mAb or isotype control mAb was administered via i.p. injection on days −1, 0 and 1 of the S. aureus skin inoculation. Of note, for P. aeruginosa Xen41, this i.d. inoculation resulted in mortality of 40% of the untreated mice, whereas none of the mice treated with Q-VD-OPH succumbed to the infection (FIG. 8E).

Measurement of total lesion size. Total lesion size (cm²) was measured from digital photographs of the back skin of anesthetized mice (2% isoflurane) using ImageJ software and a millimeter ruler as a reference.

In vivo whole animal bioluminescence and fluorescence imaging. In vivo bioluminescence imaging (BLI) to provide an approximation of the in vivo bacterial burden (when used in conjunction USA300 LAC::lux, Xen20 or Xen41 bioluminescent bacterial strains) and in vivo fluorescence imaging (FLI) (when used in conjunction with ASC-Citrine mice) were performed on anesthetized mice (2% isoflurane) using a Lumina III in vivo imaging system (IVIS) (PerkinElmer). For in vivo BLI, data are presented on a color scale overlaid on a grayscale photograph of the mice and quantified within a 1×10³ pixel circular region of interest (ROI) as total flux (photons/s). ASC-Citrine FLI signals were measured using a 488 nm excitation wavelength and a 507 nm emission wavelength and an exposure time of 0.5 seconds. In vivo FLI data are presented on a color scale overlaid on a grayscale photograph of the mice and quantified within a rectangular ROI as total radiant efficiency ([photons/s]/[mW/cm²]).

Ex vivo CFU enumeration. Skin tissue homogenates were obtained by performing a 10-mm lesional skin punch biopsy (Acuderm) and homogenizing each specimen (Pro200 Series homogenizer; Pro Scientific) in Reporter Lysis Buffer (Promega) containing protease inhibitor cocktail tablets (Roche Life Sciences) at 4° C. Ex vivo CFUs were counted after plating serially diluted skin tissue homogenates overnight on TSA plates.

Bacterial growth curve kinetics. Bacterial broth cultures of S. aureus USA300 LAC::lux, S. pyogenes strain Xen20 or P. aeruginosa strain Xen41 were prepared as described above. After overnight culture, the cultures were diluted 1:100 in their respective growth media. The bacterial cultures were either incubated with vehicle (Veh, DMSO: PBS) or various logarithmic concentrations of Q-VD-OPH (10 μg/mL, 100 μg/mL and 1000 μg/mL) in a total volume of 200 μL. The bacterial growth (OD₆₀₀) and bioluminescence (Lum) (as a measure of bacterial metabolism as the lux operon produces light in response to aldehydes produced during normal bacterial metabolism) were measured in triplicate for 10 hours cultures at 37° C. and measurements recorded at 20 minutes intervals in a Gen5 plate reader (BioTek).

Protease activity assays. The protease activity assays were performed by using the EnzChek Gelatinase/Collagenase Assay Kit and EnzChek Elastase Assay Kit (ThermoFisher) according to the manufacturer's instructions. Briefly, various concentrations (1, 10, or 100 ng) of Staphopain A (sspP), Staphopain B (sspB) or Streptopain (speB) (CUSABIO) with or without Q-VD-OPH (10, 100, 1000 μg/ml) were incubated with 1 μg of DQ gelatin, or DQ elastin (ThermoFisher) in the supplied digestion buffer in 96-well black plates (Corning, N.Y.) for 20 hours. Also, positive controls with Collagenase (0.4 U/mL) and Elastase (0.25 U/mL), with inhibitor controls in the presence or absence of protease inhibitors 1, 10-Phenanthroline (1 mM) or N-methoxysuccinyl-Ala-Ala-Pro-Val-chloromethyl ketone (0.1 mM) were performed respectively. Relative fluorescent intensity was analyzed with a Gen5 plate reader (BioTek; excitation wavelength, 485 nm; emission excitation wavelength, 538 nm).

Bone-marrow derived macrophage isolation and culture conditions. Bone-marrow derived macrophages (BMDMs) were obtained by differentiating bone marrow progenitors obtained from the tibias and femurs of 8- to 12-week-old C57BL/6 WT mice in RPMI 1640 complete media containing 20 ng/mL M-CSF (Sigma-Aldrich) for 7 days at 37° C. and 5% CO₂ in a humidified incubator, replacing media every 3 days. The purity of BMDMs was determined by flow cytometry and these cultures contained 96.64% CD11b⁺ BMDMs. BMDMs were then replated in 96-well plates at 2.5×10⁵ cells/mL for all experimental assays.

Mouse neutrophil isolation and culture conditions. Mouse neutrophils were obtained from the bone marrow of 8- to 12-week-old C57BL/6 mice WT mice by anti-Ly6G MACs magnetic bead separation according to the manufacturer's protocols (Miltenyi Biotec, Inc.). The purity of the mouse neutrophils was determined by flow cytometry and these cultures contained 97.5% Ly6G^{hi}CD11b⁺ neutrophils. The neutrophils were cultures in 96-well plates at 1.5×10⁵ cells/mL containing RPMI complete media for all experimental assays.

BMDMs and neutrophils cultures with S. aureus stimulation±caspase inhibitors. A previously described in vitro stimulation of BMDMs and neutrophils with live S. aureus was employed (9). Murine BMDMs or neutrophils were cultured in RPMI 1640 complete media at a density of 2.5×10⁵ and 1.5×10⁵ cells per 200 μL/well in a 96-well plate for BMDMs and neutrophils, respectively. These cell cultures were incubated with live S. aureus at a multiplicity of infection (MOI) of bacteria to cells of 5:1 at 37° C. and 5% CO₂ and gentamicin (20 mg/mL) was added at first 1 hour and the cultures continued for a total of 6 hours. In some experiments, specific inhibitors were added alone or in combination to the cultures at the same time as S. aureus, including the caspase inhibitors Z-WEHD-FMK (100 μM), Z-DQMD-FMK (10 μM), Z-IETD-FMK (100 μM), Z-LEHD-FMK (100 μM), Wadelolactone (20 μM), Q-VD-OPH (10 μg/ml) or Emricasan (9 μg/ml). A positive control for necroptosis was performed by culturing BMDMs or PMNs at 37° C. and 5% CO₂ for 8 hours with SMAC mimetic (IAP Antagonist) (100 nM) (Sigma-Aldrich), recombinant mouse TNF (20 ng/mL) and Z-VAD-FMK (20 μM) (R&D Systems).

Histology. Skin punch biopsy specimens (10 mm) were collected on day 3, fixed in formalin (10%) and embedded in paraffin. Sections (4 μm) were mounted onto glass slides and stained with hematoxylin and eosin (H&E) and Gram stain and digitally scanned by the Johns Hopkins Reference Histology Laboratory, according to guidelines used for clinical specimens. The images were analyzed using Aperio ImageScope (v12.4) image analysis software (Leica Biosystems). Dermonecrosis area (mm) was determined by manually measuring the width of dermonecrosis observed in each H&E section. The abscess area was determined by manually outlining the neutrophilic abscess in each H&E stained section and image analysis calculation of the area. Bacterial band width (mm) was determined by manually measuring the width of the bacterial band in each Gram-stained section.

Serum IL-1β and TNF levels. Serum IL-1β and TNF protein levels (pg/mL) were measured from serum collected on days 1 and 3 after S. aureus skin inoculation±Q-VD-OPH treatment using Bio-Plex protein assays and normalized to total protein, according to the manufacturer's recommendations (Bio-Rad).

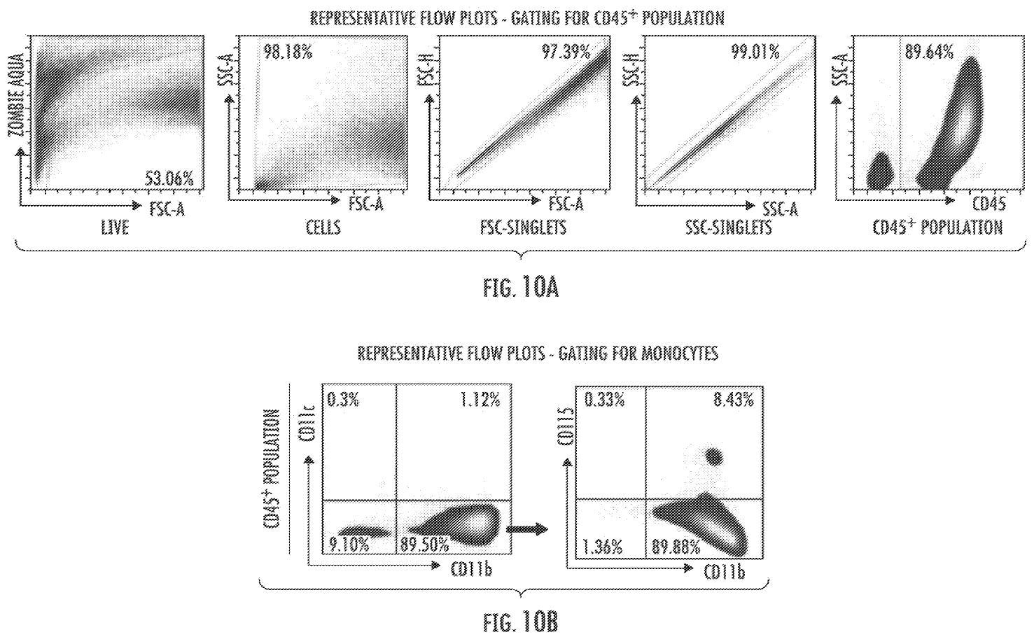

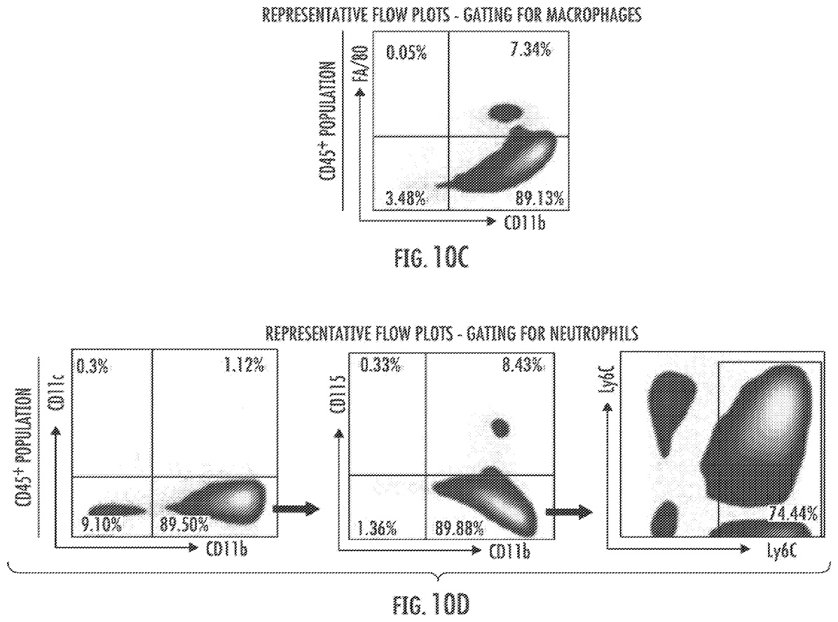

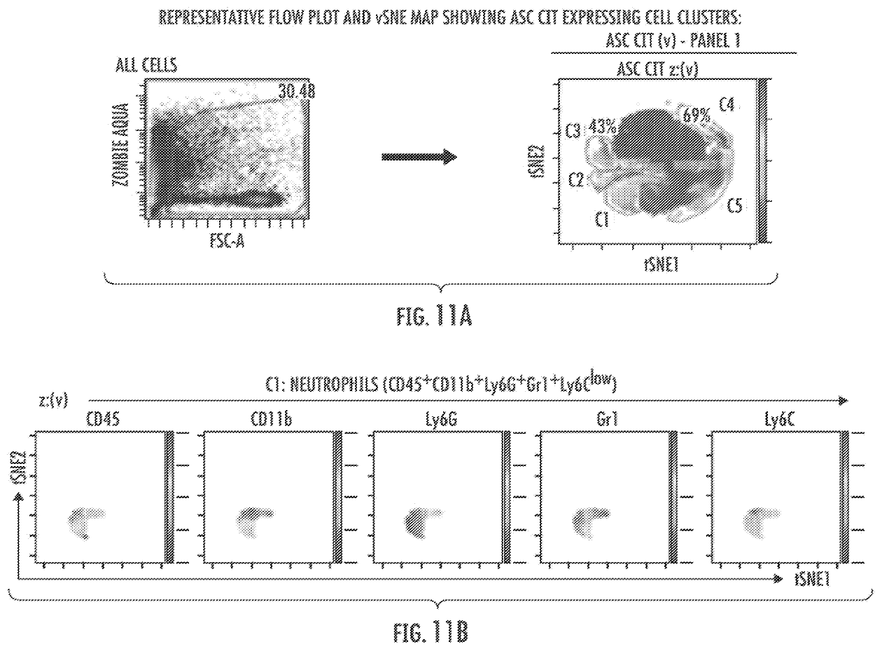

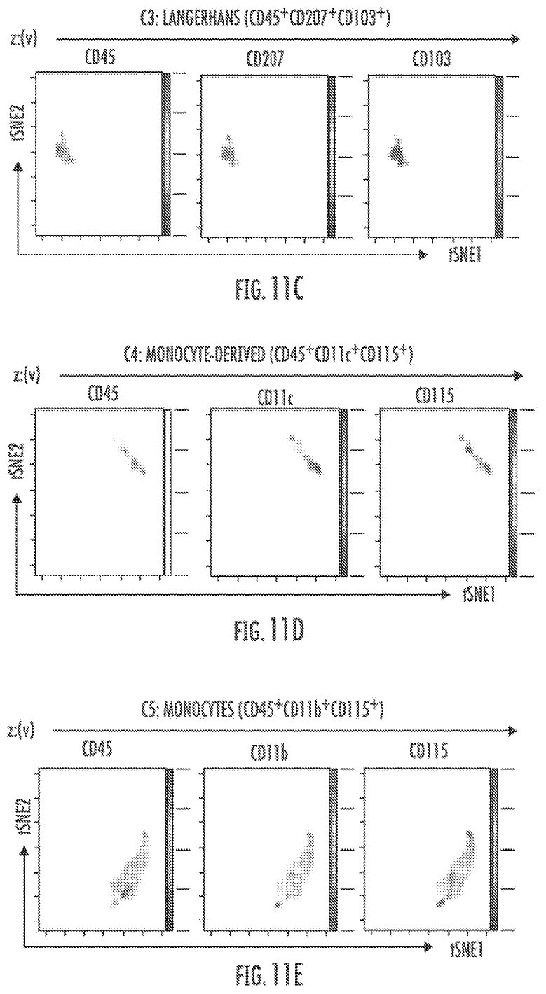

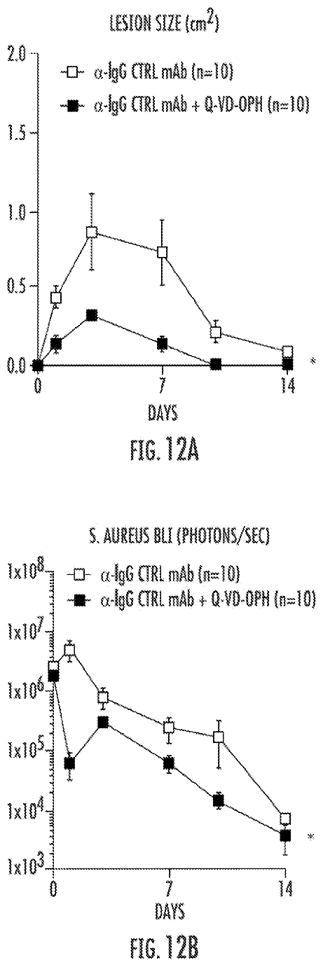

Flow cytometry. 10-mm skin punch biopsies were collected, minced and enzymatically digested in 3 mL RPMI containing 100 ng/mL DNaseI (Sigma-Aldrich) and 1.67 Wunsch U/ml Liberase TL (Roche) for 1 hour at 37° C. and shaken at 140 rpm. Single-cell suspensions were obtained after filtering the digested samples through a 40-nm cell filter using a 3 mL syringe plunger and cells were then washed in RPMI. The single-cell suspension was washed in Wash Buffer (Invitrogen) and resuspended in Annexin-V Binding Buffer and stained with Annexin-V stain (BD Biosciences). The stained cells were washed in Annexin-V Binding Buffer. The single-cell suspension was incubated with TruStain fcX (Biolegend) to block Fc receptor binding and resuspended to label with mAbs against cell surface markers (Supplemental Table S2). The cell surface markers included CD45, CD11b, CD11c, CD115, CD207, Ly6C, Ly6G and F4/80 in Hanks Balanced Salt Solution (HBSS) with 2% Calf Serum and 5 mM Hepes along with Brilliant Stain Buffer (BD Biosciences). The cells were washed with PBS and stained for viability (Zombie Aqua Fixable Viability Kit-BioLegend). The surface labeled cells were fixed in the BD Cytofix/Cytoperm Buffer kit (BD Biosciences). The cells were further labeled for intracellular pMLKL, which was detected via anti-pMLKL mAb that was biotinylated with Biotin conjugation/Fast/Type A kit (Abcam), as per manufacturer's instructions, along with other intracellular mAbs against IL-1β and TNF (Supplemental Table 51). Respective IgG isotype (Supplemental Table S2) and streptavidin controls for the intracellular mAb labeling were performed in each experiment. The mAb-labeled cells were then washed in intracellular staining buffer and resuspended in Stabilizing Fixative (BD Biosciences). Cell acquisition was performed on the BD LSRFortessa flow cytometer (BD Biosciences) and data were analyzed using Cytobank software (Cytobank). For immunophenotyping of monocytes, macrophages and neutrophils, cells were first gated on live cells, singlets and CD45⁺ cells (pan-leukocyte marker), CD11b⁺CD11c⁻ and CD115⁺ (monocytes), CD11b⁺F4/80⁺ (macrophages) and CD11b⁺ CD11c⁻ cells and Ly6G^{hi}Ly6C^{int/low} cells (neutrophils) (FIG. 10). For immunophenotyping of Langerhans cells and monocyte-derived dendritic cells (DCs), cells were first gated on CD45+ and then CD207⁺CD103⁻ (Langerhans cells) and CD45⁺CD11c⁺CD115⁺ monocyte-derived DCs (FIG. 11), modified from previously described methods (61). The absolute numbers of the respective cell populations were calculated as (Total number of live cells X [% of cell population/100]).

Pulse Shape Analysis (PuLSA) Assay. ASC-Citrine Speck analysis was performed using the PuLSA assay, as previously described (29) and according to the flow cytometry gating strategy (FIG. 11). The single-cell suspension from skin punches (as described above) was washed with PBS and stained for viability (Zombie Aqua Fixable Viability Kit-BioLegend). The live/dead stained cells were incubated with TruStain fcX (Biolegend) to block Fc receptor binding and resuspended for labeling with mAbs against surface markers (see Supplemental Table S3), in Hanks Balanced Salt Solution (HBSS) with 2% Fetal Calf Serum and 5 mM Hepes along with Brilliant Stain Buffer (BD Biosciences). The stained cells were washed in HBSS Buffer and resuspended in Stabilizing Fixative (BD Biosciences). Cell acquisition was performed on the BD LSR Fortessa flow cytometer (BD Biosciences) and data were analyzed using Cytobank software (Cytobank). The cells were first gated on live cells and singlets were processed for high-dimensional computational flow cytometry analysis to identify cell populations with ASC-Citrine expression, including neutrophils (CD45⁺CD11b⁺Ly6G^{hi}Gr1⁺Ly6C^{low}), Langerhans cells (CD45⁺CD207⁺CD103⁻), monocyte-derived dendritic cells (DCs) (CD45⁺CD11c⁺CD115⁺) and monocytes (CD45⁺CD11b⁺CD115⁺) (FIG. 11). ASC speck data are presented as Total ASC-Citrine (% of ASC Citrine/Live cells) and total ASC-Specks.

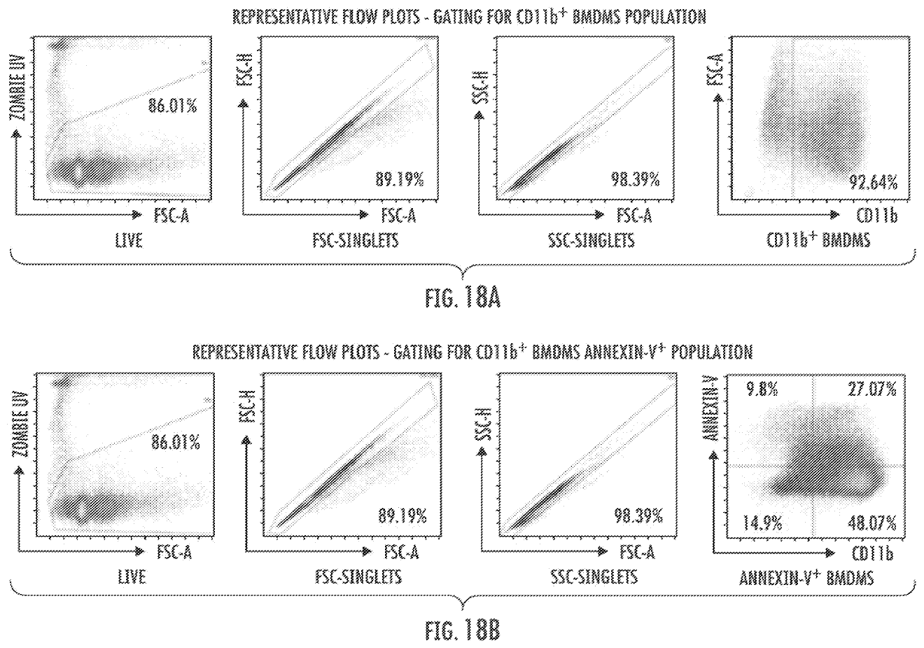

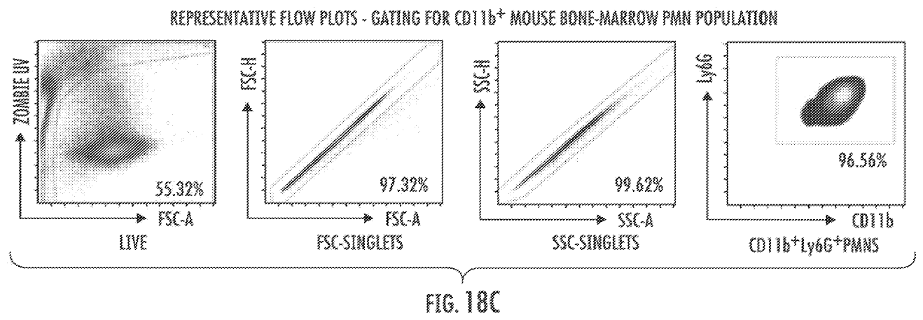

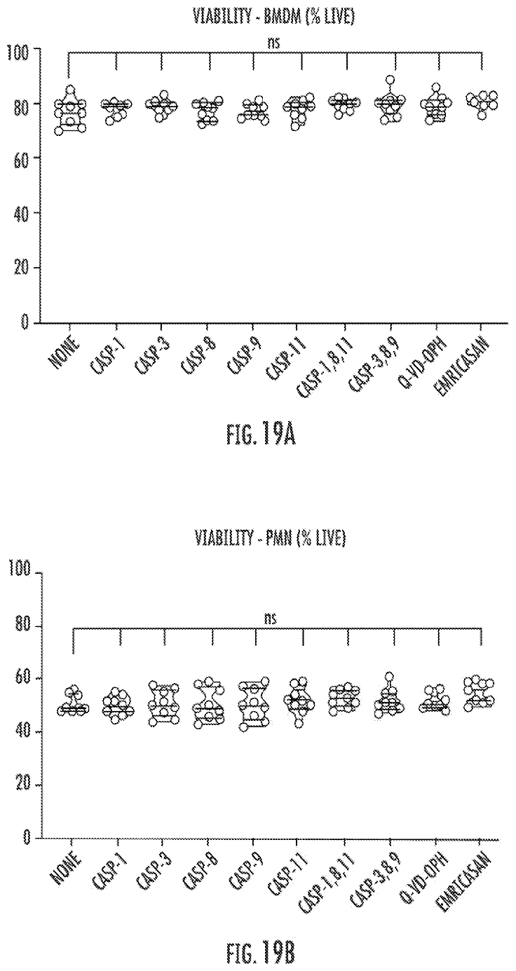

Flow cytometry for BMDMs and neutrophils for in vitro experiments. Cultured BMDMs or neutrophils were washed in Wash Buffer (Invitrogen) and resuspended in Annexin-V Binding Buffer and stained with an Annexin-V-FITC stain (BD Biosciences). The stained cells were washed in Annexin-V Binding Buffer. The single-cell suspension was incubated with TruStain fcX (Biolegend) to block Fc receptor binding and resuspended to label with mAbs against cell surface markers. The cell surface markers included CD11 b (clone BV786; BD Biosciences) for BMDMs and Ly6G-PE-Cy7 (BD Biosciences) for neutrophils in Hanks Balanced Salt Solution (HBSS) with 2% Calf Serum and 5 mM Hepes. The cells were washed with PBS and stained for viability (Zombie UV Fixable Viability Kit; BioLegend). The surface labeled cells were fixed in the BD Cytofix/Cytoperm Buffer kit (BD Biosciences). The cells were further labeled for intracellular TNF-APC (BD Biosciences). Respective IgG isotype controls for the intracellular mAb labeling were performed in each experiment. The mAb-labeled cells were then washed in intracellular staining buffer and resuspended in Stabilizing Fixative (BD Biosciences). Cell acquisition was performed on the BD LSRFortessa flow cytometer (BD Biosciences) and data were analyzed using Cytobank software (Cytobank). For gating BMDMs and neutrophils for Annexin-V and TNF intracellular expression, BMDMs and neutrophils were first gated on live cells, singlets and CD11b⁺ cells (BMDMs) and CD11b⁺Ly6G^{hi} cells (neutrophils) (Supplemental FIG. 18).

Western blot analysis. 1 mL or 200 mL of ice cold 1×RIPA buffer (Sigma-Aldrich) containing 1× of Halt protease and phosphates inhibitor cocktail (ThermoFisher) was added to skin punch biopsy specimens (10 mm) collected on day 1 or cultured BMDMs or PMNs cells. The skin samples were homogenized, and cell lysates were incubated on ice for 30 minutes and the centrifuged at 14,000 g for 20 minutes at 4° C. Protein concentration was determined by Modified Lowry Protein Assay (ThermoFisher). An equal amount of protein was run on 4-12% Bolt Bis-Tris Gel (Invitrogen) and electrotransferred onto polyvinylidene difluoride membrane (Bio-Rad). Membranes were blocked with Blocker BSA or SuperBlock Blocking Buffer (ThermoFisher), incubated overnight at 4° C. with primary antibodies, including pMLKL, MLKL, ICAD or β-actin (Abcam) (Supplemental Table S4). HRP-conjugated secondary antibodies were incubated for 1 hour at room temperature. Images were acquired using a ChemiDocXRS (Bio-Rad). The PVDF membrane was stripped with Restore Plus Western Blot stripping buffer (ThermoFisher) between individual protein detection.

Protein blot analysis. 7.5-10 ng of sspB, sspB or speB were incubated with or without 10 mg/mL of Q-VD-OPH for 2 hours at 37° C. and the protein were resolved on 4-12% Bolt Bis-Tris Gel (Invitrogen) and electrotransferred onto PVDF membrane (Bio-Rad). The membrane was stained with MemCode Reversible Protein Stain Kit (ThermoFisher). Digital photographs and images were acquired and analyzed using ChemiDocXRS (BioRad).

Statistics. Data between 2 groups for longitudinal comparisons (lesion sizes and in vivo BLI) were compared using a 2-way ANOVA and for single comparisons (ex vivo CFU and flow cytometry data at specific time points) were compared using a 2-tailed unpaired Student's t test. Data for longitudinal comparisons across multiple groups (≥3 groups) were compared using a 2-way ANOVA multiple comparisons test. P-values from multiple comparisons were adjusted by the step-up Bonferroni method to control the overall family-wise error rate. Data are presented as mean±standard error of the mean (SEM) and violin plots are expressed with the median and the inter-quartile range (ICR). Kaplan-Meier survival curves were compared using a Log-rank Mantel-Cox test. All statistical analyses were calculated using Prism (version 9) software (GraphPad; La Jolla, Calif.) for macOS (v11) and R statistical program (v4.0.2) with ggplot2 package (v3.3.3). P values <0.05 were considered statistically significant.

### Results

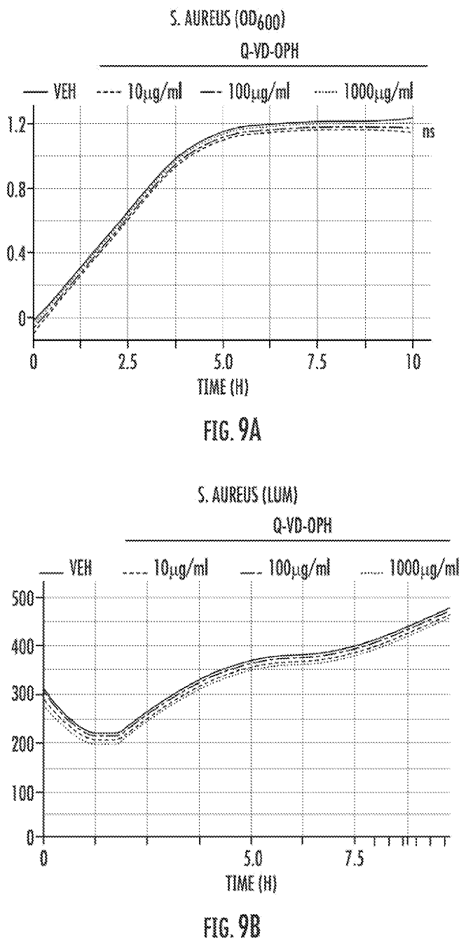

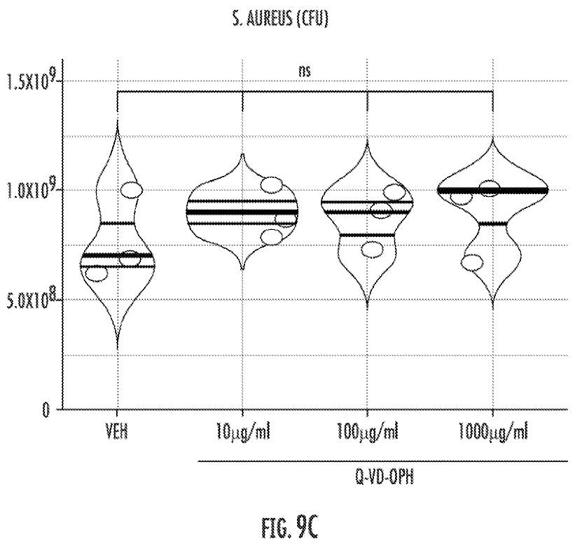

Q-VD-OPH treatment has marked efficacy against a CA-MRSA skin infection in mice. To determine whether the pan-caspase inhibitor Q-VD-OPH has any efficacy as a non-antibiotic, host-directed immunotherapy, a CA-MRSA skin infection mouse model was employed (25,26). The model involves the intradermal (i.d.) inoculation of a bioluminescent CA-MRSA strain (USA300 LAC::lux) in the backs of WT C57BL/6 mice and the bacterial burden was monitored noninvasively and longitudinally with in vivo bioluminescence imaging (BLI). The in vivo BLI closely approximates ex vivo CFU isolated from the infected skin at various time points after inoculation (R²=0.9996 (27)). WT mice were untreated (WT-Untreated) or treated with a single dose of the vehicle (DMSO+PBS; WT-Vehicle) or Q-VD-OPH (WT-QVD-OPH) intraperitoneally (i.p) at 4 hours following the CA-MRSA inoculation (FIG. 1A-1E), which was the same timing used to evaluate efficacy of orally and subcutaneously administered antibiotics in mice (27). Remarkably, Q-VD-OPH treatment rapidly and substantially reduced skin lesion sizes (FIG. 1A, 1C), bacterial burden (as measured by in vivo BLI signals [FIG. 1B, 113] and ex vivo CFU enumeration on day 3 [FIG. 1E]), compared with vehicle or untreated mice. Since no significant differences between vehicle and untreated mice were observed, untreated mice were used as the comparison control group in all subsequent experiments. It should be mentioned that the efficacy of Q-VD-OPH against the S. aureus skin infection in vivo was not due to direct antibacterial activity against S. aureus growth as incubation of Q-VD-OPH in broth cultures with logarithmic concentrations of Q-VD-OPH (i.e., 10, 100 and 1,000 μg/mL) resulted in no differences in in vitro absorbance, bioluminescence and CFU, compared with broth cultures incubated with vehicle alone (FIG. 9A-9C). Importantly, the 1,000 μg/mL concentration of Q-VD-OPH is more than 2-fold greater than the 20 mg/kg i.p. dose that was administered to the mice prior to the in vivo pharmacokinetic distribution of the Q-VD-OPH throughout the blood, tissues and organs of the mice.

Skin biopsies were obtained at 3 days following the bacterial inoculation and histologic sections were stained with hematoxylin and eosin (H&E) or Gram stain and analyzed by image analysis (FIG. 1F-1J). On H&E-stained sections, Q-VD-OPH resulted in decreased dermonecrosis width and abscess area, compared with untreated mice (FIG. 1F, 1H, 1I). On Gram-stained sections, Q-VD-OPH resulted in decreased bacterial band length, compared with untreated mice (FIG. 1G, 1J). Collectively, Q-VD-OPH had therapeutic efficacy in this mouse model of CA-MRSA infection with decreased skin lesions and bacterial burden.

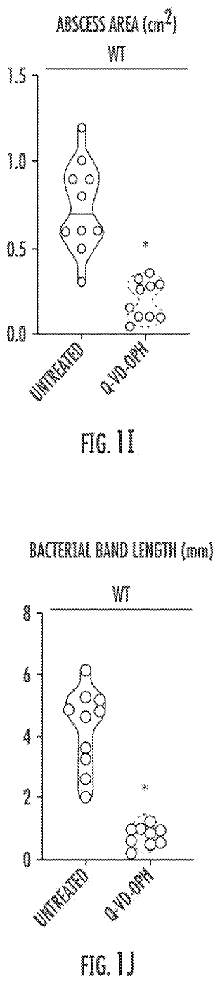

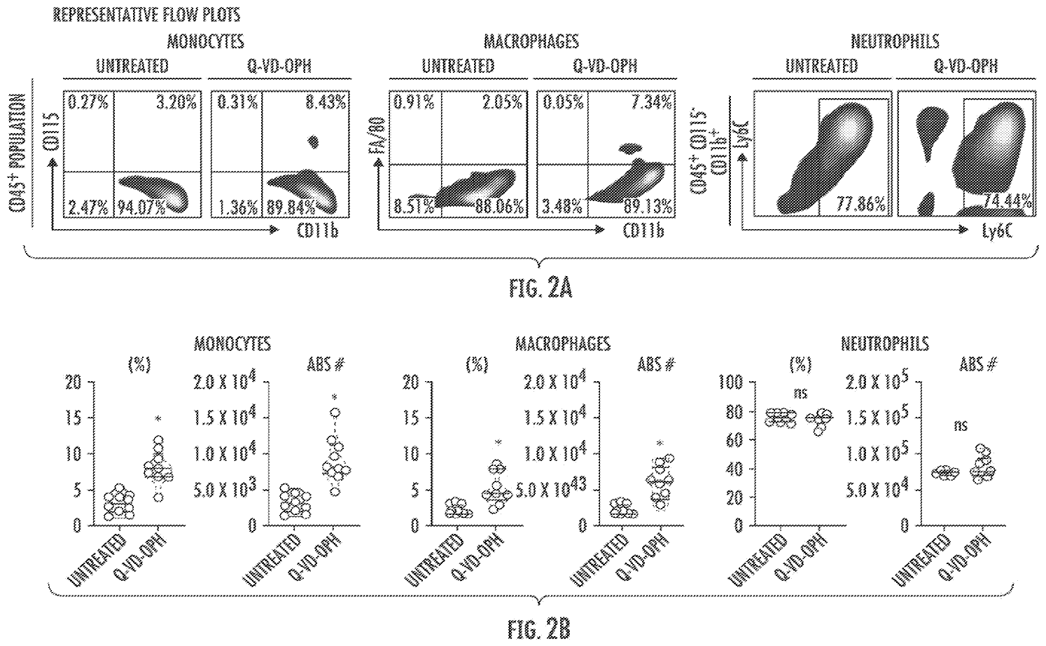

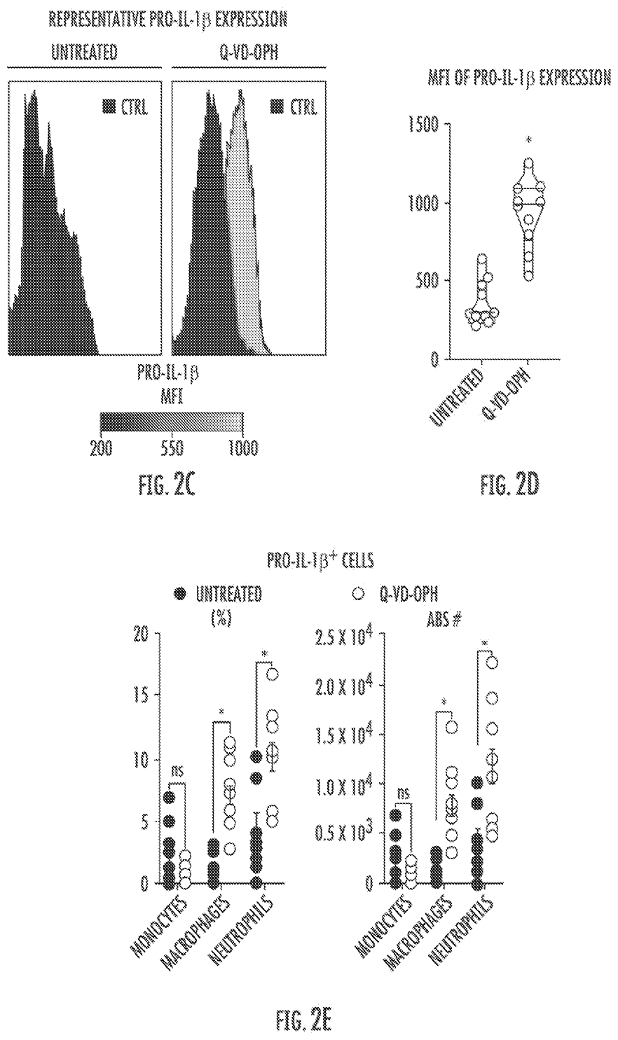

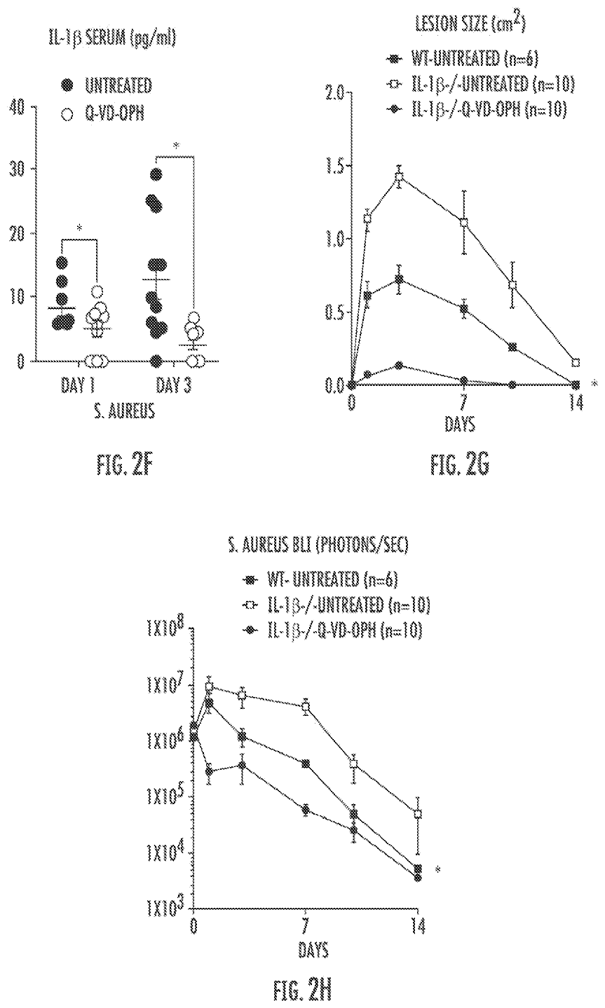

Q-VD-OPH treatment increases monocyte/macrophage recruitment and its efficacy is independent of IL-1β activity. In monocytes, macrophages and neutrophils, S. aureus pore-forming toxins (e.g., α-toxin, PVL and LukAB) induce NLRP3/ASC inflammasome-mediated caspase-1-dependent processing of pro-IL-1β to the active and secreted form of IL-1β in vitro (6-8), which induces neutrophil recruitment and host defense against the skin and other types of S. aureus infections in vivo (9-11). Therefore, the percentages and absolute numbers of monocytes (CD45⁺CD11b⁺CD115⁺), macrophages (CD45⁺CD11b⁺F4/80⁺) or neutrophils (CD45⁺Ly6G^{hi}Ly6C^{int/hi}) from biopsies of infected skin on day 1 following CA-MRSA skin inoculation were evaluated in Q-VD-OPH treated and untreated mice (flow cytometry gating strategy is shown in the online supplemental data, FIG. 10A-10D). The percentages of monocytes and macrophages were statistically increased. In contrast, there were no differences in the percentages and absolute numbers of neutrophils in Q-VD-OPH treated mice, compared with untreated mice (FIG. 2A, 2B). The degree and the percentage and absolute numbers of cells that produced intracellular pro-IL-1β (pro-IL-1β⁺ cells) was also evaluated, and Q-VD-OPH treatment resulted in increased mean fluorescence intensity (MFI) and percentages of total leukocytes (CD45⁺pro-IL-1β⁺) (FIG. 2C, 2D), especially in macrophages and neutrophils but not monocytes (FIG. 2E) in Q-VD-OPH treated mice, compared with untreated mice. Curiously, there were decreased serum levels of IL-1β on days 1 and 3 in Q-VD-OPH treated mice compared with uninfected mice (FIG. 2F). Given the contradiction of increased intracellular pro-IL-1β (in neutrophils and monocytes) but reduced serum IL-1β levels, we next determined whether IL-1β was involved in the efficacy of Q-VD-OPH by performing the CA-MRSA skin infection mouse model in IL-1β^{-/-} mice (FIG. 2G, 2H). In IL-1β^{-/-} mice, Q-VD-OPH had marked efficacy with substantially reduced skin lesions and in vivo BLI signals compared with untreated mice, despite the larger skin lesions and higher bacterial burden seen in untreated IL-1β^{-/-} mice. Therefore, the mechanism of efficacy of Q-VD-OPH was independent of IL-113 activity.

Q-VD-OPH inhibits ASC speck formation. To determine whether Q-VD-OPH treatment affected NLRP3/ASC inflammasome-triggered caspase-1 activation (upstream of pro-IL-1β processing and mature IL-1β secretion) as described to occur in S. aureus skin and other types of infections (9, 25, 26, 28), we investigated for the formation of ASC specks, which are required for NLRP3 inflammasome assembly. The CA-MRSA skin infection mouse model was performed in ASC-Citrine mice (29) in the presence or absence of treatment with a single dose of Q-VD-OPH, i.p. at 4 hours. The bacterial burden was evaluated by in vivo BLI imaging (FIGS. 3A, 3B) and ASC-speck formation was evaluated by in vivo fluorescence (FLI) imaging at 6 hours following Q-VD-OPH administration (FIGS. 3C, 3D). There was no difference in the in vivo BLI imaging signals between Q-VD-OPH treated versus untreated ASC-Citrine mice (FIGS. 3A, 3B). However, the in vivo FLI signals revealed that Q-VD-OPH virtually inhibited all in vivo ASC speck formation, which conversely was increased in untreated mice (FIGS. 3C, 3D). To confirm these in vivo results, single cell suspensions from biopsies of CA-MRSA-infected skin on days 0 and 1 were evaluated in Q-VD-OPH treated and untreated mice. ASC specks were assessed with a pulse shape analysis (PuLSA) assay using high-dimensional computational flow cytometry, as previously described (29) (flow cytometry gating strategy is shown in FIG. 11). As shown on the viSNE plots and quantification for the total percentage and absolute numbers of ASC-Citrine expression (without evaluating for ASC Specks), the percentage of total ASC-Citrine expression in live cells increased from days 0 to 1 but was not significantly different between Q-VD-OPH treated and untreated mice (FIGS. 3E, 3F). However, as shown on the viSNE plots and quantification for the total percentage and absolute numbers of ASC-Specks, Q-VD-OPH treated mice had a substantially reduced percentage of cells with ASC-Specks (FIGS. 3G, 3H) as compared with untreated mice, corroborating the in vivo findings in FIGS. 3C, 3D. Finally, and similarly to IL-1β^{-/-} mice (FIGS. 2G, 2H), Q-VD-OPH maintained high efficacy in ASC^{-/-} mice, with substantially reduced skin lesions and in vivo BLI signals compared with untreated mice, despite the larger skin lesions and higher bacterial burden seen in untreated ASC^{-/-} mice (FIGS. 3I, 3J). Collectively, although Q-VD-OPH inhibited assembly of the ASC inflammasome complex, Q-VD-OPH still had efficacy against the CA-MRSA infection in the absence of ASC activity (in ASC^{-/-} mice), indicating that the efficacy of Q-VD-OPH was independent of ASC inflammasome complex formation.

Q-VD-OPH efficacy does not involve caspases 1 and 11 or Gasdermin D-mediated pyroptosis. Since Q-VD-OPH treatment inhibited ASC speck formation (FIG. 3A-H) and is known to inhibit caspases 1 and 11 (23, 24), which are key caspases activated by ASC-dependent inflammasomes, we determined whether Q-VD-OPH treatment had any efficacy in mice deficient in caspases 1 and 11. The CA-MRSA skin infection mouse model was performed in caspase-1^{-/-} mice, caspase-11^{-/-} mice or mice deficient in both caspases 1 and 11 (caspase-1/11^{-/-} mice) in the presence or absence of treatment with a single dose of Q-VD-OPH i.p. at 4 hours (FIG. 4A-4F). It should be noted that the skin lesion sizes and in vivo BLI signals of caspase-1^{-/-} (FIG. 4A, 4B), caspase-11^{-/-} (FIG. 4C, 4D) and caspase-1/11^{-/-} mice (FIG. 4E, 4F) were not significantly different than those of WT Untreated mice, indicating that deficiency of caspases 1 and 11 alone or in combination did not impact host defense against the CA-MRSA skin infection. However, Q-VD-OPH treatment resulted in markedly decreased skin lesion sizes and in vivo BLI signals in caspase-1^{-/-} (FIG. 4A, 4B), caspase-11^{-/-} (FIG. 4C, 4D) and caspase-1/11^{-/-} (FIG. 4E, 4F) mice. Therefore, the efficacy of Q-VD-OPH was not dependent on the activity of caspases 1 and 11 (alone or in combination).

Caspases 1 and 11 are known to activate Gasdermin-D (GSDMD), which is a critical pore-forming protein that is important for IL-1β secretion from cells and for inflammasome-mediated pyroptosis (30). To determine whether the efficacy of Q-VD-OPH occurred through inflammasome-mediated pyroptosis dependent on Gasdermin D, the CA-MRSA skin infection mouse model was performed in Gasdermin D^{-/-} mice (FIG. 4G, 4H). Similar to IL-1β^{-/-}, caspase-1^{-/-}, caspase-11^{-/-} and caspase-1/11^{-/-} mice, Q-VD-OPH maintained efficacy in Gasdermin D^{-/-} mice with substantially reduced skin lesions and in vivo BLI signals compared with untreated mice, despite the larger skin lesions and higher bacterial burden in untreated Gasdermin D^{-/-} mice. Combined, these data indicate that Q-VD-OPH efficacy was independent of the activity of Gasdermin D.

Q-VD-OPH decreases apoptotic neutrophils/monocytes and increases necroptotic macrophages. Given that Q-VD-OPH treatment still had efficacy in mice deficient in IL-1β, caspases 1 and 11 or Gasdermin-D, the mechanism of efficacy of Q-VD-OPH did not involve IL-1β activity or inflammasome-mediated pyroptosis. Therefore, we evaluated whether the efficacy of Q-VD-OPH involved other cell death mechanisms that occur during S. aureus infections, including apoptosis (12-14, 31-34) and necroptosis (15, 16, 35-37). CA-MRSA skin infection was performed in WT mice±treatment with a single dose of Q-VD-OPH i.p. at 4 hours post-inoculation and single-cell suspensions from biopsies of infected skin on day 1 were evaluated for any changes in the percentages of cells undergoing early apoptosis (FIG. 5A-5C) or necroptosis (FIG. 5D-G). Q-VD-OPH treatment led to significantly decreased percentages and absolute numbers of early apoptotic cells of total leukocytes (CD45⁺ Annexin V⁺), monocytes and neutrophils but not macrophages, compared with untreated mice (FIG. 5A-5C) (see flow cytometry gating strategy [FIG. 10A-10D]). Next, the degree and percentages and absolute numbers of cells undergoing necroptosis (pMLKL⁺) were also evaluated. Q-VD-OPH treatment led to significantly greater MFI, percentages and absolute numbers of total leukocytes and macrophages but not monocytes or neutrophils, compared with untreated mice (FIG. 5E-5G). Taken together, Q-VD-OPH treatment led to reduced apoptosis in neutrophils and monocytes and increased necroptosis in macrophages.

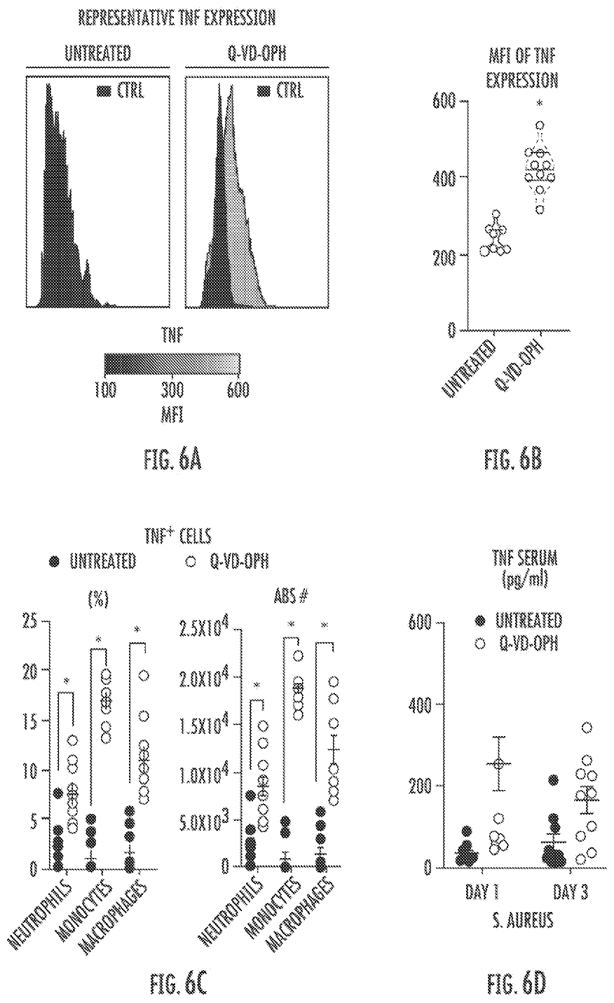

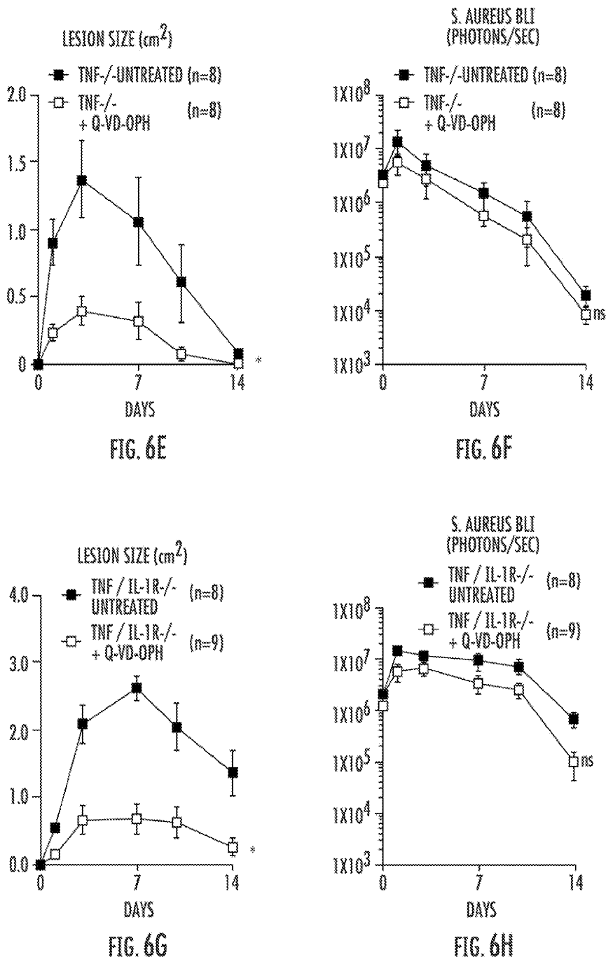

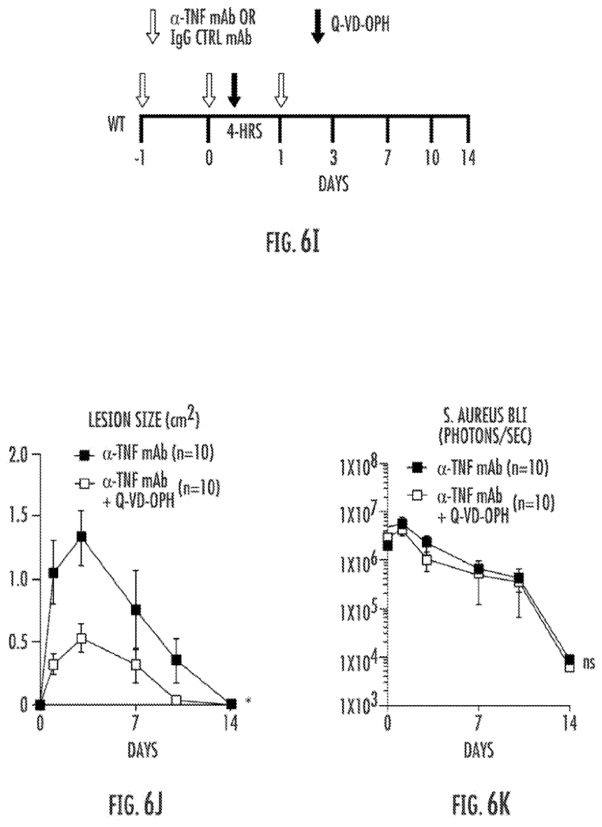

Q-VD-OPH induced bacterial clearance is dependent upon TNF activity. As Q-VD-OPH is known to inhibit necroptosis in the setting of hepatitis C infection (21) and TNF can initiate necroptotic cell death (38), a role for TNF in the efficacy of Q-VD-OPH was evaluated. The CA-MRSA skin infection mouse model was performed in WT mice±treatment with a single dose of Q-VD-OPH i.p. at 4 hours post-inoculation and single-cell suspensions from biopsies of infected skin on day 1 were evaluated for any changes in the percentages and the absolute number of cells producing TNF (TNF⁺). Q-VD-OPH treatment led to significantly greater MFI, percentages and absolute number of total leukocytes (CD45⁺TNF⁺), monocytes, macrophages and neutrophils, compared with untreated mice (FIG. 6A-6C) (see flow cytometry gating strategy [FIG. 10A-10D]). Correspondingly, Q-VD-OPH resulted in increased serum TNF protein levels on days 1 and 3, compared with untreated mice (FIG. 6D).

Next, to determine whether the Q-VD-OPH-induced increased levels of TNF in immune cells and the serum of the treated mice contributed to the therapeutic mechanism of action, the CA-MRSA skin infection mouse model was performed in TNF^{-/-} mice (FIG. 6E, 6F). In TNF^{-/-} mice, although Q-VD-OPH treatment resulted in significantly smaller lesion sizes than untreated mice, there was no significant difference in vivo BLI signals between Q-VD-OPH treated and untreated mice. Therefore, Q-VD-OPH lacked efficacy in reducing the bacterial burden in TNF^{-/-} mice, indicating that the efficacy of Q-VD-OPH involved TNF-mediated clearance of the infection. Additionally, to evaluate if this role of TNF in the efficacy of Q-VD-OPH occurred independently of the host defense role of IL-1β, the same experiment was performed in mice deficient in both TNF and IL-1R (TNF/IL-1R^{-/-} mice) (FIG. 6G, 6H). Similarly, in TNF/IL-1R^{-/-} mice, although Q-VD-OPH treatment resulted in significantly smaller lesion sizes than untreated mice, there was no significant difference in in vivo BLI signals between Q-VD-OPH treated and untreated mice. These results are in stark contrast to the marked efficacy of Q-VD-OPH in IL-1β^{-/-} mice (FIG. 2G, 2H), further implicating TNF (rather than IL-1β) as the predominant inflammatory cytokine mediating the efficacy of Q-VD-OPH against the CA-MRSA skin infection. Finally, to confirm that these results were not due to a developmental defect in TNF^{-/-} mice, the CA-MRSA skin infection mouse model was performed in WT mice in the presence of a systemically administered anti-TNF neutralizing mAb (αTNF mAb) (on days −1, 0 and 1) (FIG. 6I). In WT mice administered with an αTNF mAb, Q-VD-OPH treatment resulted in significantly smaller lesion sizes than untreated mice, but there was no significant difference in in vivo BLI signals between Q-VD-OPH treated and untreated mice (FIG. 6J, 6K), findings virtually identical to TNF^{-/-} mice (FIG. 6E, 6F). As a negative control and as expected, in WT mice administered with an isotype control mAb, Q-VD-OPH resulted in decreased in vivo BLI signals, compared with untreated mice (FIG. 12). These results indicate a key role for TNF in mediating the decreased bacterial burden in response to Q-VD-OPH treatment.

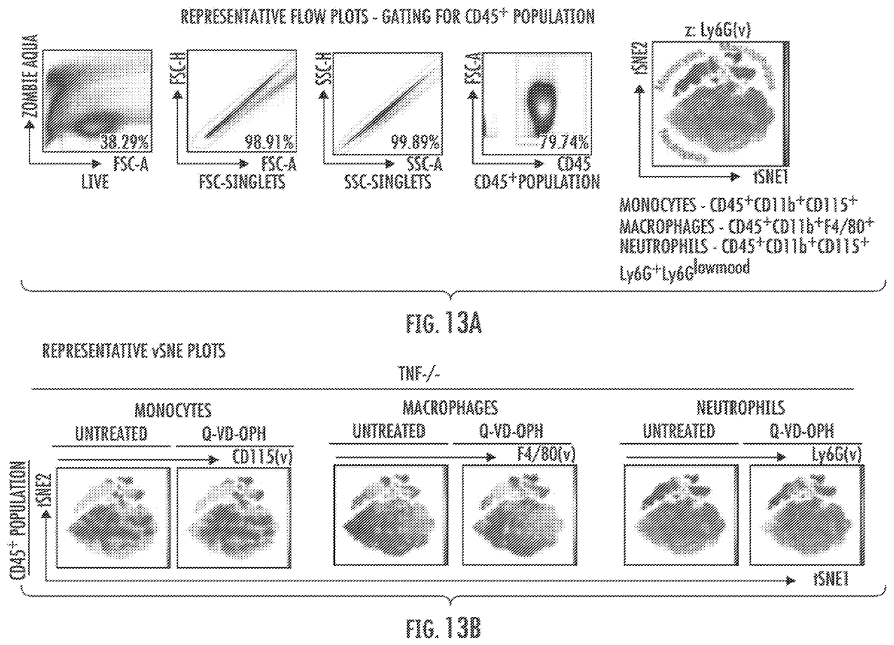

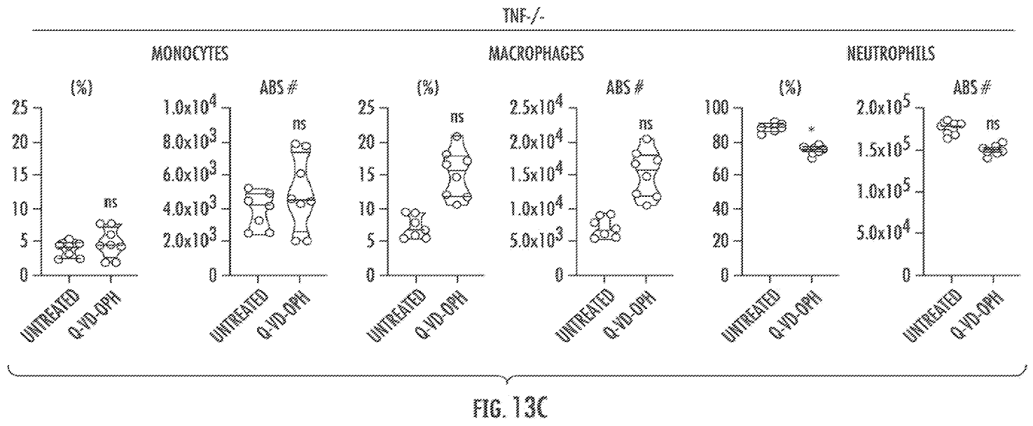

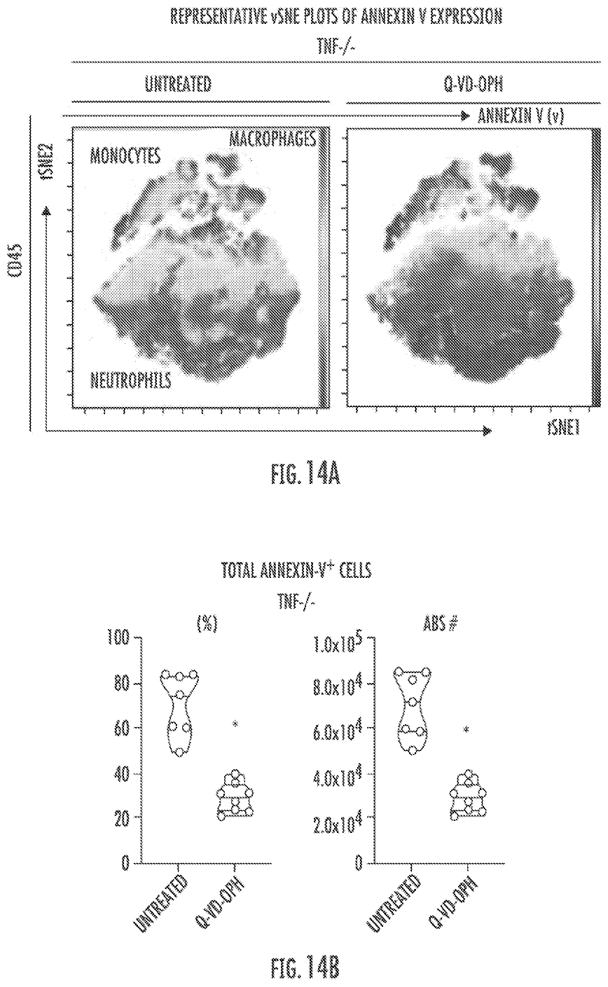

Q-VD-OPH reduces neutrophil influx and does not increase necroptosis in absence of TNF. To evaluate the immune response of Q-VD-OPH treatment in TNF^{-/-} and WT mice (FIG. 2A-2B), a CA-MRSA skin infection mouse model was performed with or without Q-VD-OPH treatment in TNF^{-/-} mice. The percentages and the absolute number of neutrophils (CD45⁻Ly6G^{hi}Ly6C^{int/hi}) from biopsies of infected skin on day 1 following CA-MRSA skin inoculation in Q-VD-OPH treated mice were significantly reduced when compared to untreated mice or Q-VD-OPH treated WT mice (FIG. 13B-13C vs. FIG. 2A-2B) (see flow cytometry gating strategy [FIG. 10A-10D and FIG. 5A]). Interestingly, there were no increase in the percentages and the absolute number of monocytes in Q-VD-OPH treated TNF^{-/-} mice (FIG. 13B-13C) compared with Q-VD-OPH treated WT mice (FIG. 2A-2B). However, the percentages and the absolute number of macrophages were statistically increased in Q-VD-OPH treated TNF^{-/-} mice (FIG. 13B-13C).

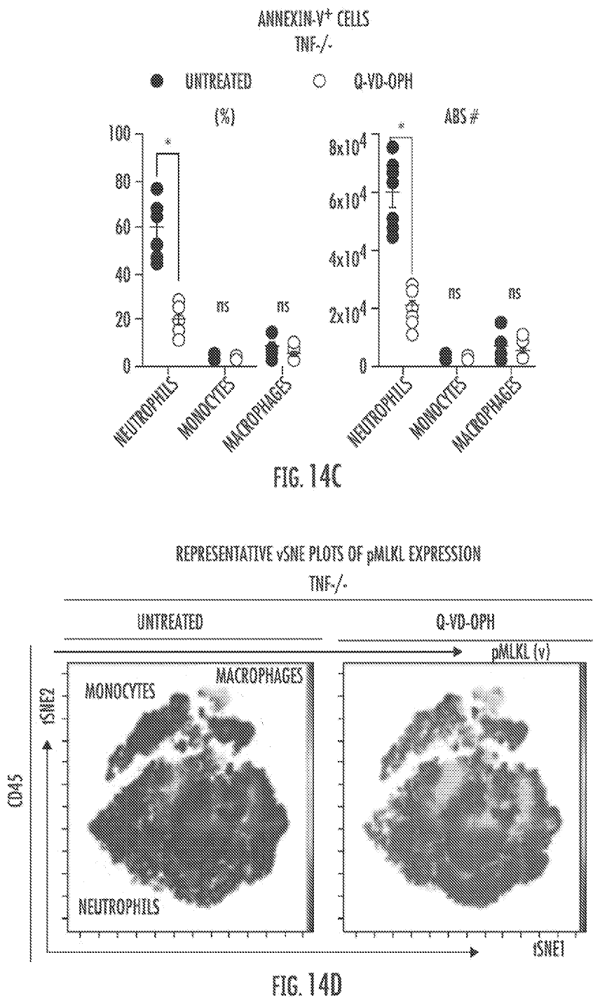

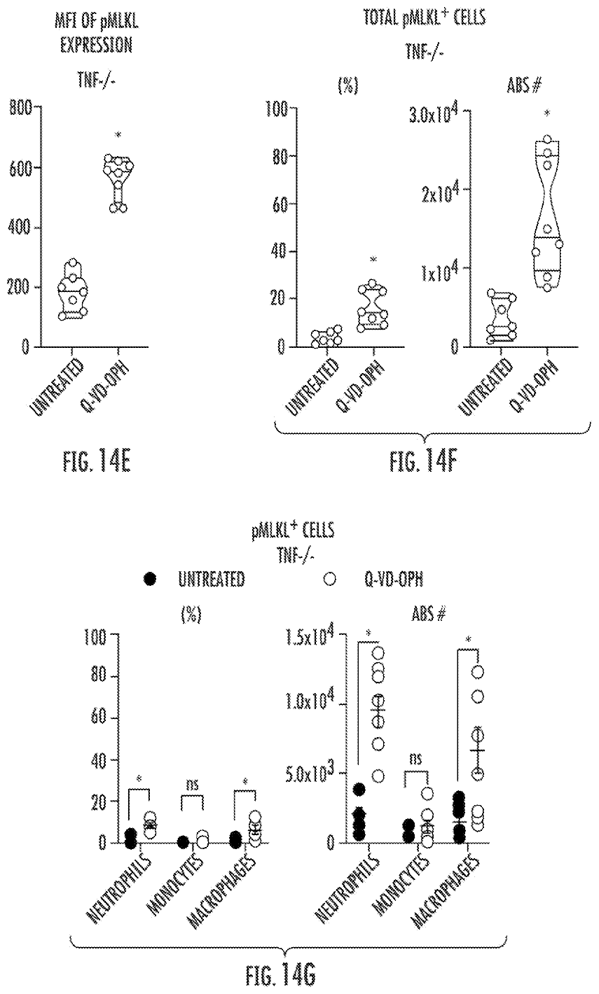

Furthermore, Q-VD-OPH treatment in TNF^{-/-} mice led to significantly decreased percentages and the absolute numbers of early apoptotic cells of total leukocytes (CD45⁺Annexin V⁺), neutrophils but not monocytes or macrophages, compared with untreated mice and Q-VD-OPH treated WT mice (FIG. 14A-14C, vs. FIG. 5A-5C) (see flow cytometry gating strategy [FIG. 10A-10D]). Next, the degree and percentages and absolute numbers of cells undergoing necroptosis (pMLKL⁺) in TNF^{-/-} mice were also evaluated. Interestingly, Q-VD-OPH treatment led to significantly greater MFI, percentages and absolute numbers of total leukocytes in TNF^{-/-} compared with TNF^{-/-} untreated mice (FIG. 14D-14F). However, the MFI, percentages and absolute numbers in Q-VD-OPH treated TNF^{-/-} mice (540, 15%, 1.5×10⁴ cells respectively) (FIG. 14E, 14F) were significantly reduced compared with Q-VD-OPH treated WT mice (2250, 81%, 1.0×10⁵ cells respectively) (FIG. 5E, 5F). Furthermore, pMLKL⁺ cells in Q-VD-OPH treated TNF^{-/-} mice were predominantly neutrophils and macrophages but not monocytes when compared with untreated mice (FIG. 14G).

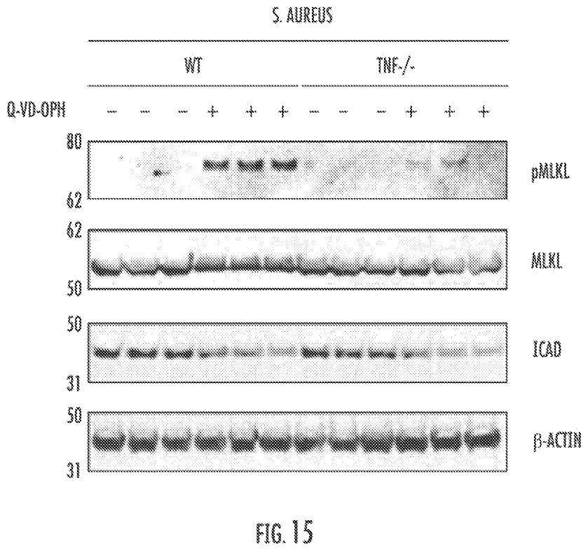

Taken together, Q-VD-OPH treatment led to reduced apoptosis in neutrophils and reduced overall necroptosis in TNF^{-/-} mice treated with Q-VD-OPH. Furthermore, western blot analysis of Q-VD-OPH treated TNF^{-/-} mice in the CA-MRSA skin infection mouse model demonstrated reduced necroptosis compared with untreated or Q-VD-OPH treated WT mice, as indicated by pMLKL and MLKL expression (FIG. 15). Furthermore, as previously observed, there was reduced apoptosis (ICAD) in the CA-MRSA skin infection mouse in both Q-VD-OPH treated WT and TNF^{-/-} mice (FIG. 15) of infected skin on day 1. Taken together, these results suggest that even with decreased apoptosis, there is a reduced influx of neutrophils at the site of infection in Q-VD-OPH treated TNF^{-/-} mice.

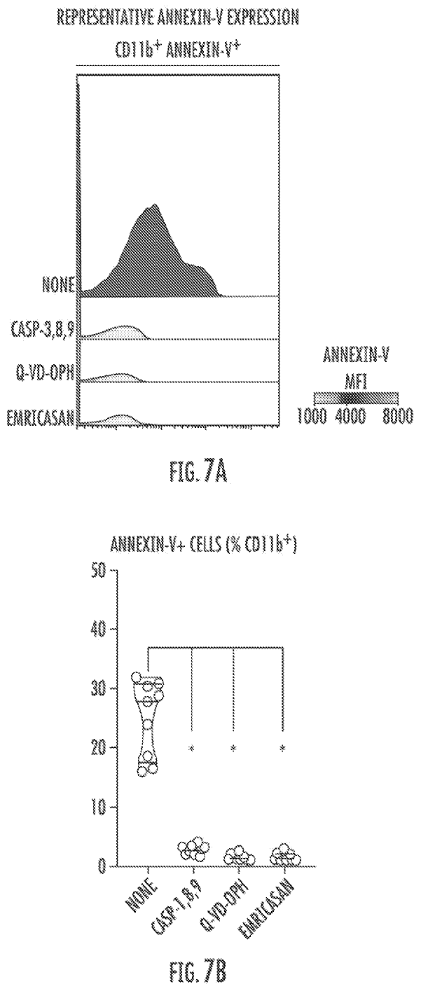

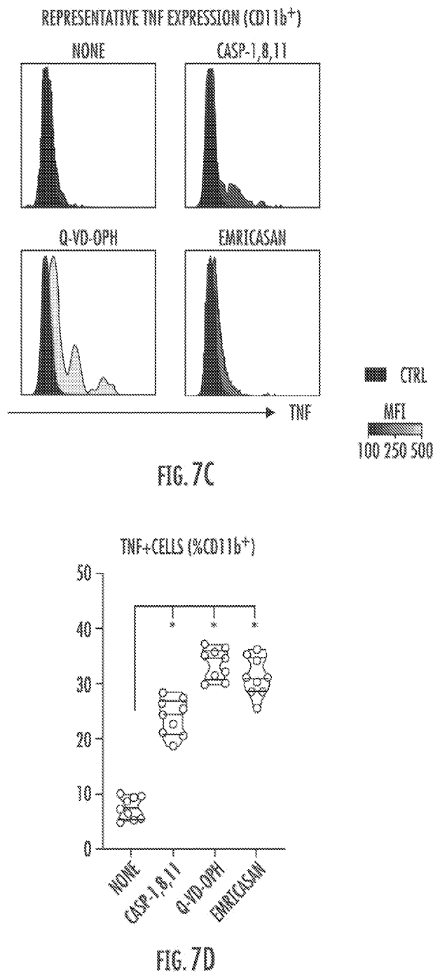

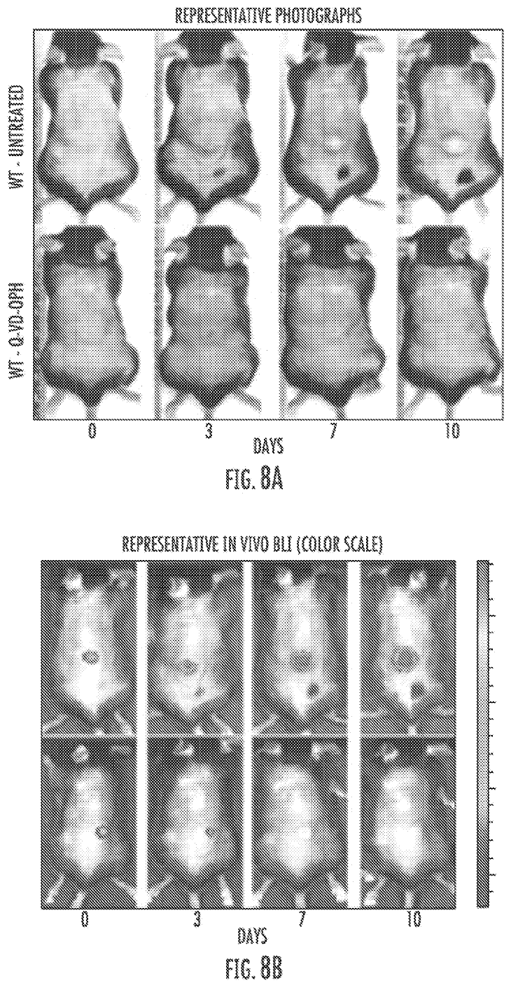

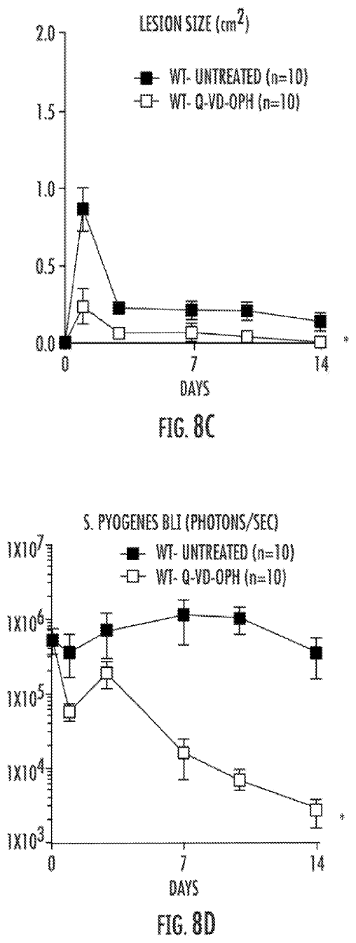

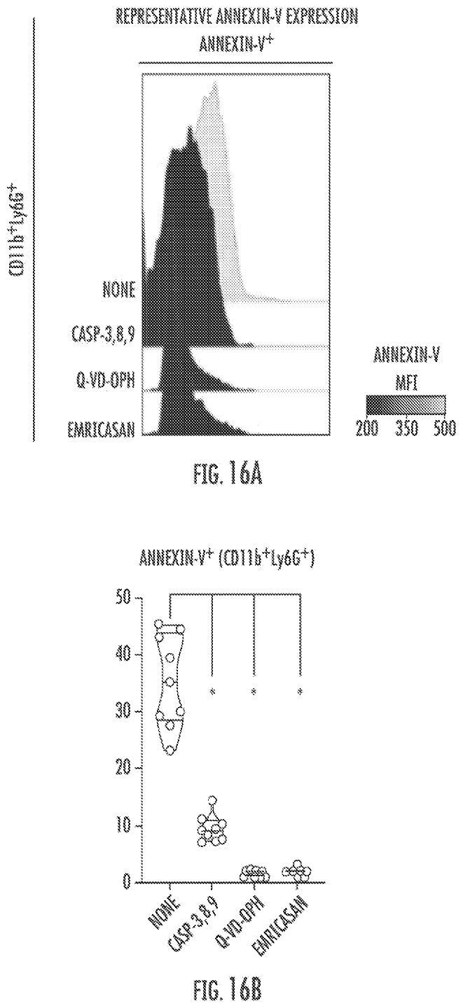

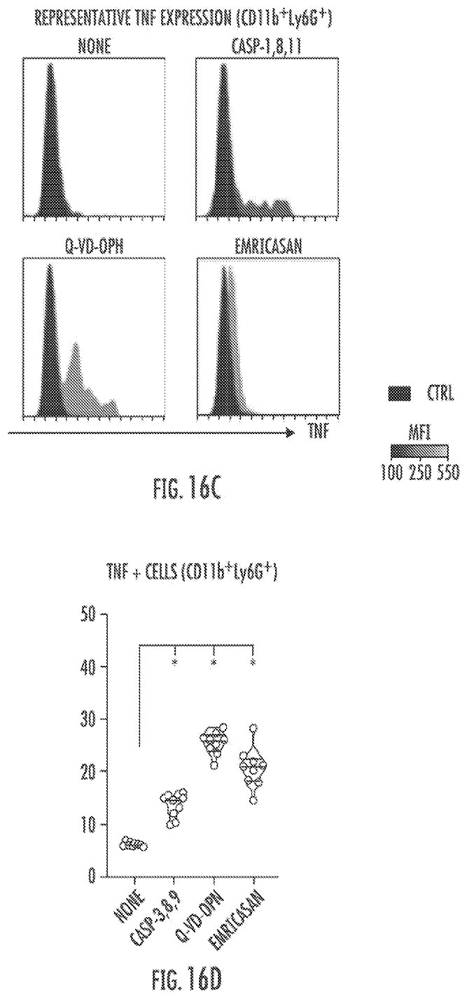

Combined caspase 3, 8 and 9 inhibition reduced apoptosis and combined caspase 1, 8 and 11 inhibition induced TNF production, similar to Q-VD-OPH or Emricasan in vitro. Since Q-VD-OPH inhibits many caspases (i.e., caspases 1, 3 and 7-12 (17, 18)), it is unclear which specific caspases that were inhibited by Q-VD-OPH led to therapeutic efficacy against the CA-MRSA skin infection in vivo. To determine which caspases were involved, we used a previously described S. aureus culture system that involved the incubation of mouse bone marrow-derived macrophages (BMDMs) (FIG. 7) or mouse neutrophils (FIG. 16) with live S. aureus for 6 hours with gentamicin added after the first hour of culture (to kill all of the S. aureus) (9). In these cultures, inhibitors for caspases 1, 3, 8, 9 or 11, Q-VD-OPH, Emricasan, or no treatment (None) were included and intracellular levels of early apoptosis (Annexin-V⁺) (FIGS. 7A, 7B, 16A, 16B, 17A, 17B, and 17E, 17F) and TNF (FIGS. 7C, 7D, 16C, 16D, 17C, 17D and 17G, 17H) were evaluated by flow cytometry (as in FIGS. 5C and 6C). The gating strategy for the in vitro cultured BMDMs and PMNs is shown in FIG. 18. It should be noted that there were no significant differences in cell viability with any of the caspase inhibitors, Q-VD-OPH or Emricasan in BMDMs (˜80% viable cells) or neutrophils (˜50% viable cells), as measured by the percentage of live cells using flow cytometry (Zombie UV negative cells) (FIG. 19).

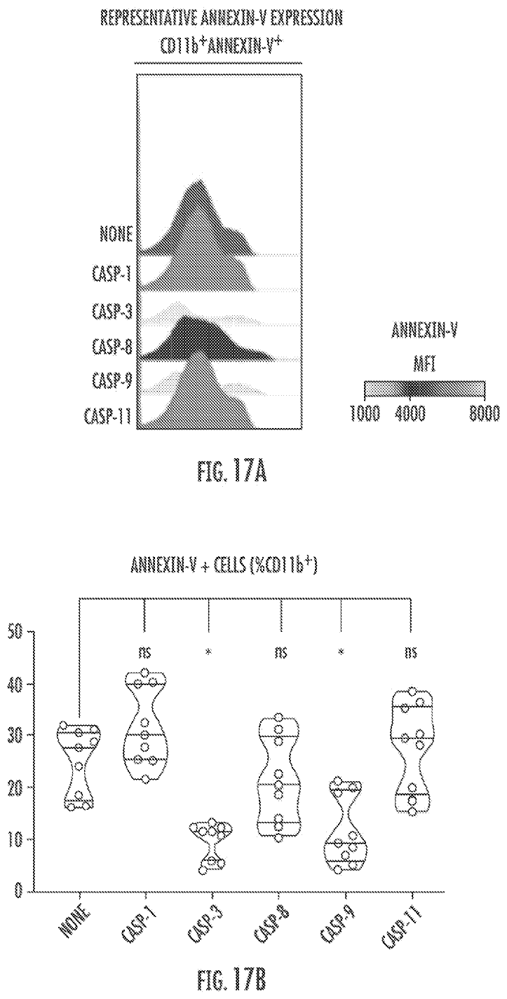

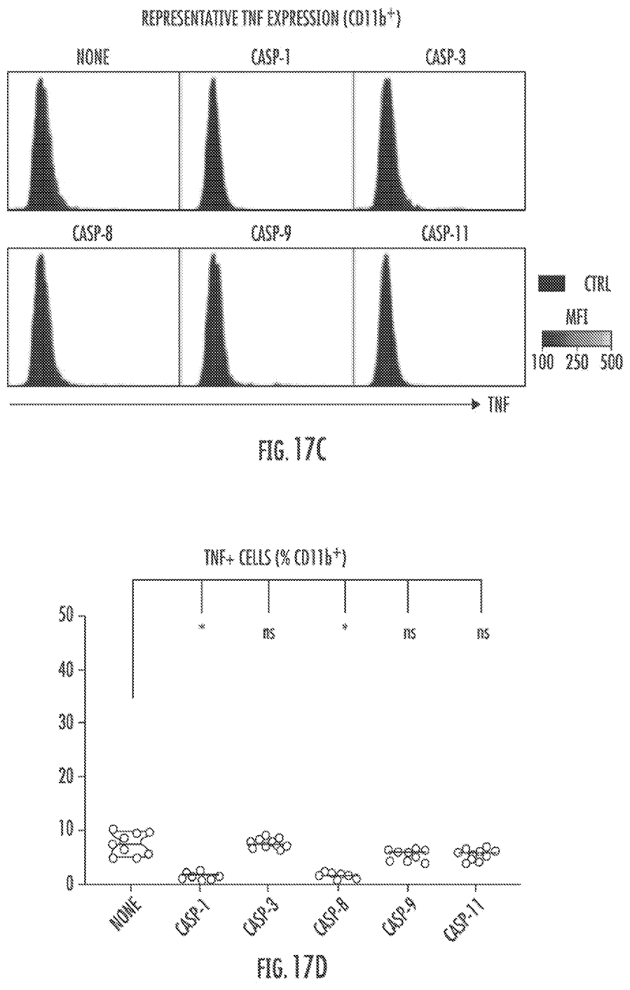

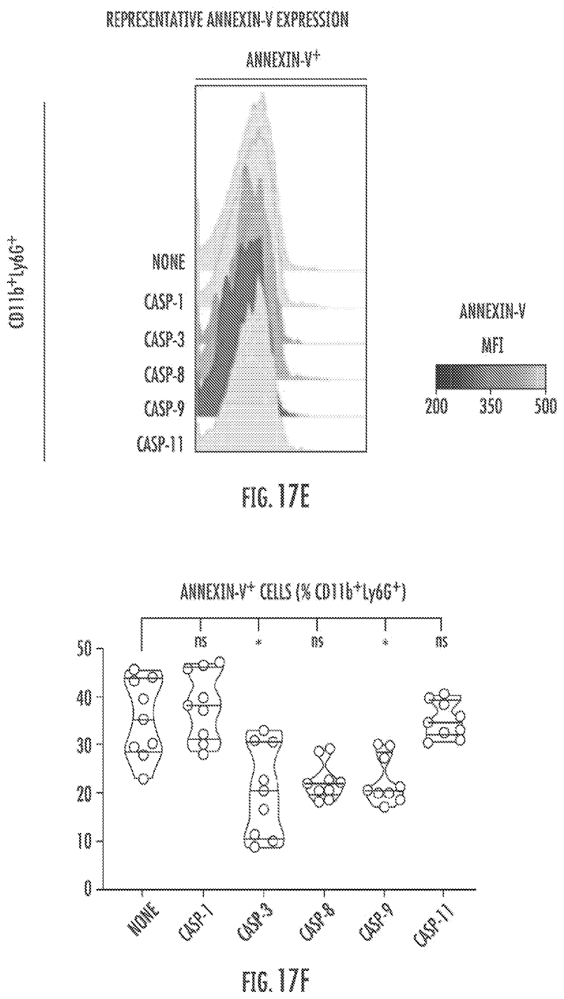

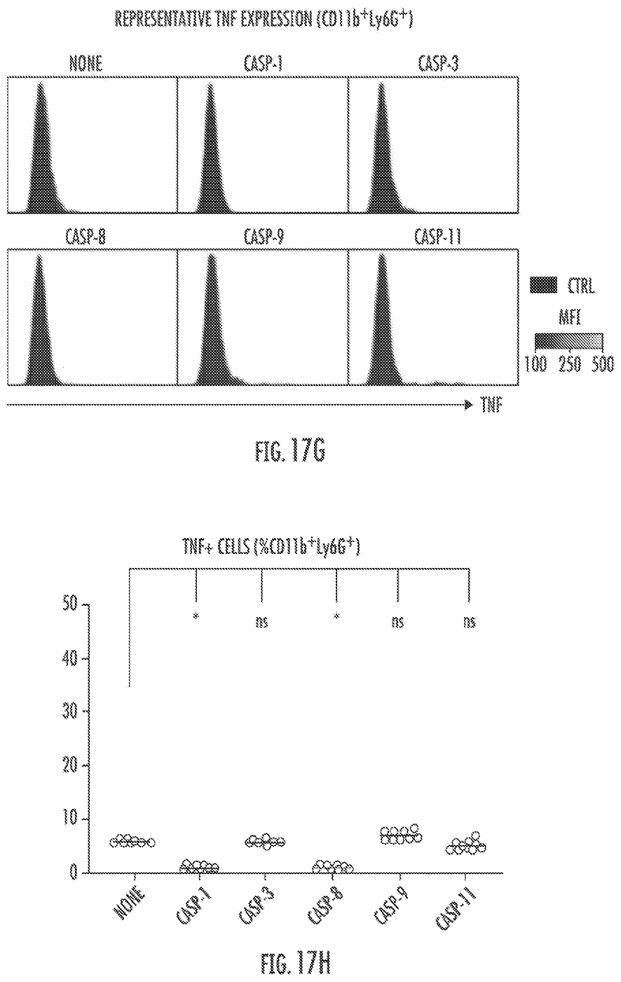

Regarding Annexin V⁺ BMDMs and neutrophils, inhibition of caspases 1 or 11 resulted in percentages of Annexin V⁺ BMDMs and neutrophils that did not significantly differ than no treatment (27.89-38.31%) (FIG. 17A, 17B and FIG. 17E, 17F), suggesting that inhibition of caspases 1 or 11 did not reduce S. aureus-induced apoptosis in vitro. In contrast, inhibition of caspases 3, 8 or 9 resulted in significantly lower percentages of Annexin V⁺ BMDMs and neutrophils compared with no treatment (P<0.05), but none fully recapitulated the low percentages of Annexin V⁺ BMDMs and neutrophils induced by Q-VD-OPH or Emricasan (<3%, for both). Since caspases 3, 7, 8 and 9 all contribute to apoptosis (30), we hypothesized that the low percentages of Annexin V⁺ cells induced by Q-VD-OPH or Emricasan might be due to combined inhibition of caspases 3, 7, 8 and 9 (30). Although there is no specific inhibitor available for caspase 7, the inhibitors of caspases 3, 8 and 9 were combined in the cultures and this led to even lower percentages of both Annexin V⁺ BMDMs (2-4%) and neutrophils (˜10%), which closely approximated the <3% Annexin V⁺ BMDMs and neutrophils observed with Q-VD-OPH or Emricasan treatment.

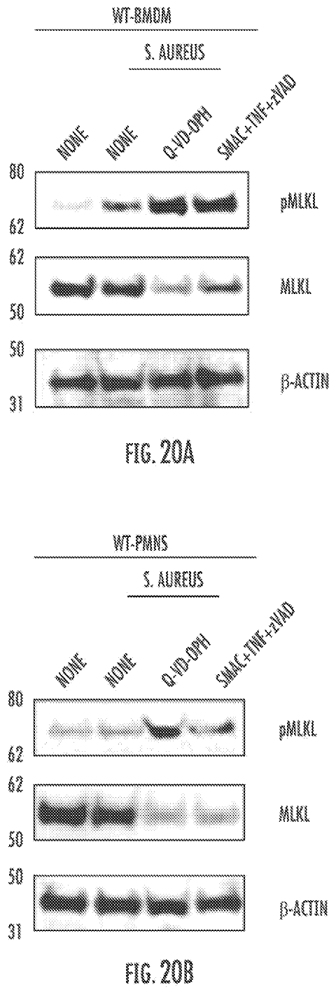

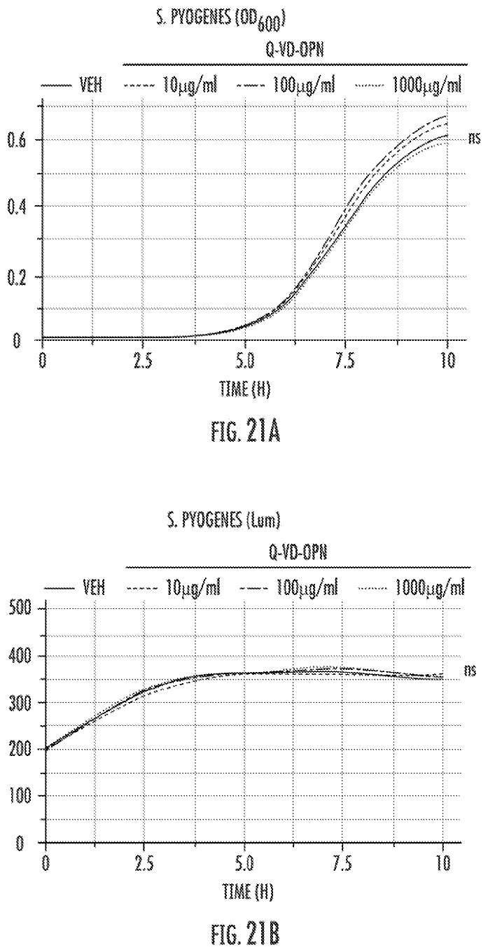

Regarding TNF⁺ BMDMs and neutrophils, inhibition of caspases 1 or 8 resulted in significantly lower percentages of TNF⁺ BMDMs and neutrophils compared with no treatment (1.29-1.6%, P<0.05% for both) (FIG. 17C, 17D and FIG. 17G, 17H). Inhibition of caspases 3, 9 or 11 resulted in percentages of TNF⁺ BMDMs and neutrophils that did not significantly differ than no treatment. Given that none of the individual caspase inhibitors recapitulated the substantially higher induction of TNF⁺ BMDMs and neutrophils induced by Q-VD-OPH treatment (33.53% and 25.47%, respectively), we hypothesized that the high percentages of TNF⁺ BMDMs and neutrophils induced by Q-VD-OPH might be due to combined activities of proinflammatory caspases implicated in TNF production. These caspases include caspases 1 and 11 (which mediate in Nlrp3/ASC/Gasdermin D-dependent pyroptosis (39) and caspase-8 (which enhances Nlrp3 activation, cleaves Gasdermin D (40) and mediates TNF-induced necroptosis (38). Therefore, inhibitors of caspases 1, 8 and 11 were combined in the cultures and this led to significantly higher percentages of TNF⁺ BMDMs (23.82%, P<0.05) and TNF⁺ neutrophils (13.49%), which closely approximated the 33.53% TNF⁺ BMDMs and 25.47% TNF⁺ neutrophils observed with Q-VD-OPH or Emricasan treatment. Furthermore, similar to S. aureus skin infection in vivo, necroptosis was increased in BMDMs and neutrophils that were treated with Q-VD-OPH, as observed by western blot analysis for pMLKL and MLKL in BMDMs and neutrophils (FIG. 20A, 20B).

Collectively, these in vitro findings indicate that the activity of Q-VD-OPH in inhibiting apoptosis of BMDMs and neutrophils was due to combined inhibition caspases 3, 8 and 9 whereas the activity of Q-VD-OPH in inducing TNF⁺ BMDMs and neutrophils was due to combined inhibition of the inflammatory caspases 1, 8 and 11. These data provide mechanistic insights into how Q-VD-OPH contributes to the inhibition of neutrophil and monocyte apoptosis (FIG. 5) and increased TNF production by monocytes, macrophages and neutrophils (FIG. 6) during the S. aureus skin infection in vivo.

Q-VD-OPH treatment also has efficacy against S. pyogenes and P. aeruginosa skin infections in mice. To determine whether Q-VD-OPH represents a non-antibiotic, host-mediated immunotherapeutic against other skin pathogens, we evaluated its efficacy against Streptococcus pyogenes (S. pyogenes) and Pseudomonas aeruginosa (P. aeruginosa), which are also common causes of bacterial skin infections in humans (4). First, a S. pyogenes i.d. skin infection model was performed with a bioluminescent S. pyogenes strain (Xen20 (41)) in the backs of WT C57BL/6 mice and a single dose of Q-VD-OPH treatment or no treatment (untreated) was administered at 4 hours and lesion sizes and in vivo BLI were measured as in FIG. 1A-1D (FIG. 7A-7D). Q-VD-OPH treatment resulted in substantially decreased skin lesion sizes (FIG. 7A, 7C) and rapidly reduced bacterial burden compared to untreated mice (FIG. 7B, 7D).

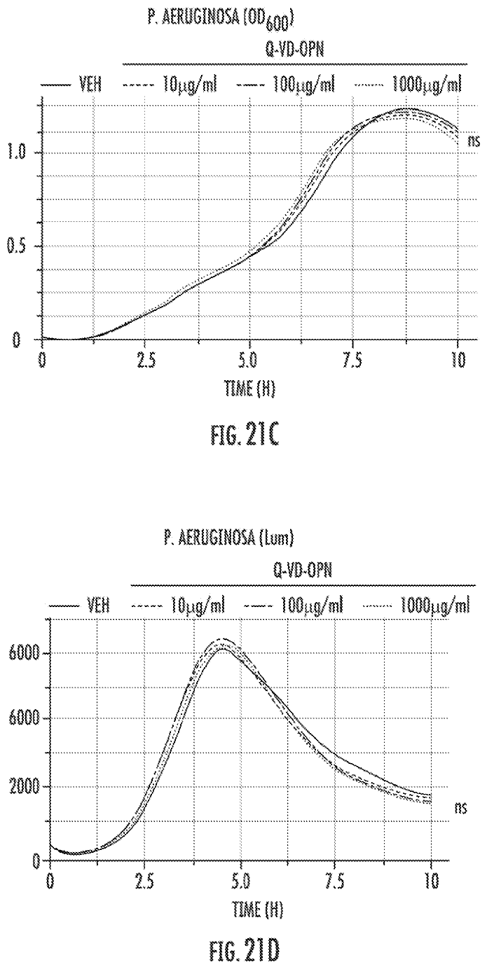

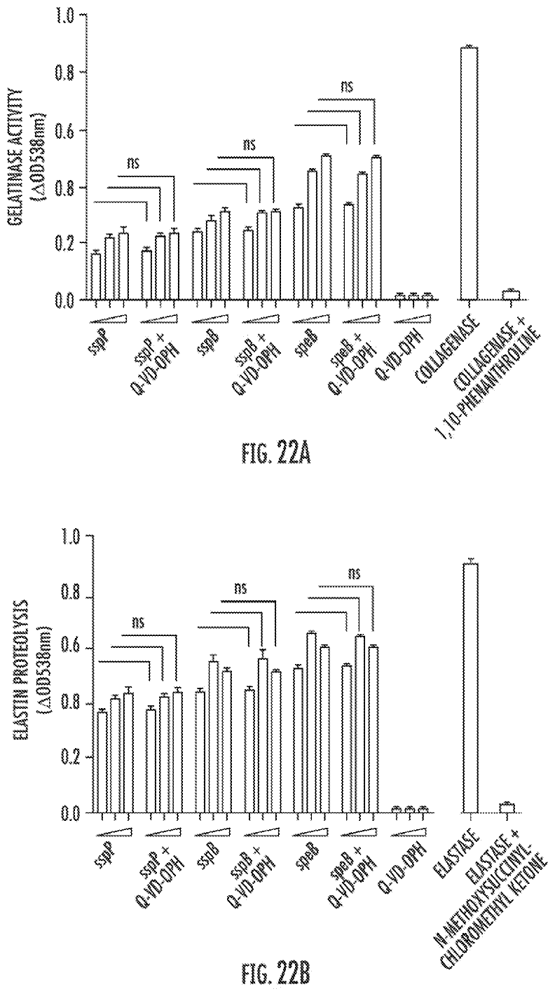

Second, a P. aeruginosa skin infection model was performed by i.d. inoculation of a bioluminescent P. aeruginosa strain (Xen41(42)) in the backs of WT C57BL/6 mice and Q-VD-OPH treatment or no treatment (untreated) was administered at 4 hours and lesion sizes and in vivo BLI were measured as in FIG. 7A-7D (FIG. 8A-8D). Q-VD-OPH treatment resulted in modest but significantly decreased skin lesion sizes (FIG. 8A, 8C) and bacterial burden, compared with untreated mice (FIG. 8B, 8D). However, in this P. aeruginosa skin infection model, the bacterial infection disseminated as seen in other initially localized infection models using Xen41(42), resulting in a mortality of 40% of the untreated mice. Remarkably, none of the mice treated with Q-VD-OPH succumbed to the infection (FIG. 8E). Taken together, Q-VD-OPH treatment had similar efficacy in another Gram-positive bacterial skin with S. pyogenes and also had therapeutic efficacy and prevented mortality against a Gram-negative P. aeruginosa skin infection. Similar to S. aureus, the in vivo efficacy of Q-VD-OPH against the S. pyogenes and P. aeruginosa skin infections was not due to an effect on bacterial growth, as S. pyogenes (FIG. 21A, 21B) or P. aeruginosa (FIG. 21C, 21D) broth cultures incubated with logarithmic concentrations of Q-VD-OPH (i.e., 10, 100 and 1,000 μg/mL) resulted in no differences in in vitro absorbance and bioluminescence, compared with cultures incubated with vehicle alone. Combined, these results indicate the potential broad therapeutic activity and innovative approach of employing pan-caspase inhibition against multiple different types of bacterial skin infections.

### Discussion

The immune mechanisms that protect against S. aureus infections have remained elusive, which has hindered the development of an effective immunotherapy or vaccine against this important human pathogen, which is becoming increasingly resistant to antibiotics (5). Herein, we sought to determine whether the potential inhibition of cell death pathways (i.e., pyroptosis, apoptosis and necroptosis) by the systemically-administered pan-caspase inhibitor Q-VD-OPH (17, 18) could promote host defense against a mouse model of CA-MRSA skin infection. A single dose of Q-VD-OPH did not impact pyroptosis but rather reduced apoptosis in neutrophils and monocytes and increased necroptosis in macrophages. This resulted in enhanced TNF production, leading to rapid bacterial clearance in the absence of antibiotics. The reduced apoptosis could be recapitulated by combined inhibition of caspases 3, 8 and 9 in vitro, whereas increased TNF production could be reproduced by combined inhibition of caspases 1, 8 and 11 in vitro, providing mechanistic insights for the efficacy of Q-VD-OPH in vivo. However, the caspase inhibitors used are known to have broader inhibitory effects (43) and given the complexities of caspases in orchestrating cell death pathways (44), future studies are warranted to fully understand the individual contributions of the caspases involved in host defense against CA-MRSA skin infections. Nonetheless, our findings provide the proof-of-concept for targeting pan-caspase inhibition as a non-antibiotic host-directed therapy against CA-MRSA skin infections and potentially other Gram-positive and Gram-negative bacterial pathogens, as observed in the S. pyogenes and P. aeruginosa skin infection models. Consistent with this possibility, Emricasan, another pan-caspase inhibitor that induces necroptosis by inhibiting caspase-8 (45), had similar efficacy as Q-VD-OPH. Importantly, responses induced by pan-caspase inhibition have revealed several new insights into the role of protective versus non-protective cell death mechanisms of neutrophils, monocytes and macrophages as well as TNF responses in host defense against CA-MRSA skin infections.

First, Q-VD-OPH had efficacy against the CA-MRSA skin infection in a mechanism independent of IL-1β, the NLRP3/ASC inflammasome and Gasdermin D-induced pyroptosis (FIGS. 1-4). This result was unexpected since IL-1β, NLRP3/ASC inflammasome, as well as a caspase-1 activity, mediate host defense, neutrophil recruitment and bacterial clearance in multiple mouse models of S. aureus infections (e.g., skin infection, brain abscesses and sepsis) (9, 25, 26, 28). Moreover, pore-forming toxins of S. aureus such as α-toxin, PVL and LukAB induce activation of human and mouse monocytes/macrophages in vitro (6-8). Nonetheless, Q-VD-OPH had marked efficacy with reduced lesion sizes and bacterial burden in mice deficient in IL-1β, ASC, caspase-1, caspase-11, both caspases 1 and 11 and in Gasdermin D. This remarkable efficacy of Q-VD-OPH occurred even with the larger skin lesions and higher bacterial burden in mice deficient in IL-1β, ASC and Gasdermin D. Since the efficacy of Q-VD-OPH did not occur through IL-1β or inflammasome-mediated pyroptosis, this prompted us to evaluate alternative host defense mechanisms that contributed to the efficacy.

Second, the efficacy of Q-VD-OPH against the CA-MRSA skin infection was found to involve reduced apoptosis of monocytes and neutrophils but not macrophages. Q-VD-OPH has known anti-apoptotic effects likely due to its inhibition of caspases 3, 7, 8 and 9 involved in apoptosis (30). Indeed, combined inhibition of caspases 3, 8 and 9 reduced the percentages of apoptotic (Annexin V⁺) BMDMs and neutrophils to a similar degree as Q-VD-OPH in vitro, providing an explanation for the reduction in apoptotic monocytes and neutrophils during the CA-MRSA skin infection in vivo. Notably, Q-VD-OPH has been evaluated as a therapeutic agent that reduces apoptotic-mediated cell death in non-infectious conditions in vivo and in vitro preclinical models of viral infection and injury (17-22). Specifically, in a simian immunodeficiency virus (SIV) infection model in rhesus macaques, Q-VD-OPH treatment promoted viral clearance that was associated with reduced T cell death (20), similar to the reduced apoptosis of neutrophils and monocytes in the CA-MRSA skin model. With respect to bacteria, Q-VD-OPH has been shown to reduce apoptosis of intestinal epithelial cells associated with barrier disruption in response to Campylobacter jejuni but not enteropathogenic Escherichia coli in vitro challenge (46, 47) and retinal cells in response to S. aureus in vivo and in vitro challenge (33). However, in all of these studies, a role for Q-VD-OPH in promoting bacterial clearance was not investigated. Therefore, the findings in the present study revealed a previously unrecognized therapeutic effect of Q-VD-OPH in host defense against S. aureus, S. pyogenes and P. aeruginosa skin infections. A potential explanation for the broad activity of Q-VD-OPH against these different bacterial species is that they are all prototypical pyogenic bacterial infections, which require effective neutrophil and monocyte/macrophage responses within an abscess to wall off the infection and promote bacterial clearance (9, 25, 26).

Third, the efficacy of Q-VD-OPH against CA-MRSA skin infection resulted in enhanced necroptotic macrophages (pMLKL⁺), suggesting that there was preserved survival of neutrophils and monocytes (i.e., less necroptosis) compared with macrophages. Prior studies have also indicated that human and mouse neutrophils and monocytes/macrophages also undergo necroptosis in response to S. aureus challenge in vitro (16, 35, 37). However, in the present study, in all of these myeloid cell types, there was enhanced TNF production and Q-VD-OPH no longer had any therapeutic effect on bacterial clearance in TNF^{-/-} mice, TNF/IL-1R^{-/-} mice and WT mice administered with an αTNF mAb (compared with untreated mice). Therefore, the ability of Q-VD-OPH to enhance TNF production and responses was essential in mediating its efficacy. Interestingly, although there was reduced apoptosis of neutrophils, Q-VD-OPH treated TNF^{-/-} mice had less neutrophil influx to the infected skin. Furthermore, necroptosis was reduced in TNF^{-/-} mice and Q-VD-OPH treated TNF^{-/-} mice compared with WT mice, suggesting an essential role of TNF in inducing necroptosis and neutrophil influx. Notably, the effect of TNF on reducing bacterial burden occurred without a reduction in skin lesion sizes, in contrast to results with Q-VD-OPH (which reduced both). Although the reason for this is unclear, Q-VD-OPH but not TNF might have prevented infection-induced cell death of keratinocytes that preserved the epidermis, leading to decreased skin lesion sizes. This protective role of Q-VD-OPH on epidermal keratinocytes will be a focus of our future research.

The protective role of TNF during S. aureus infection is becoming increasingly relevant as TNF responses have been shown to be induced and have a protective effect in mouse models S. aureus skin infections, brain abscesses, sepsis and septic arthritis (25, 26, 28, 48). Importantly, combined inhibition of caspases 1, 8 and 11 increased the percentages of TNF BMDMs and neutrophils to a similar degree as Q-VD-OPH in vitro, providing a mechanistic explanation for the increased TNF producing monocytes, macrophages and neutrophils during the in vivo S. aureus skin infection. It should be mentioned that the results with Q-VD-OPH do not preclude the findings from CA-MRSA skin infection and sepsis models performed in the context of inhibition of necroptosis (e.g., mice with deletion of MLKL or RIP3K or mice treated with RIPK1 or RIPK3 inhibitors), which resulted in high bacterial burden and enhanced inflammation (including IL-1β production) (15). Similarly, in a mouse model of CA-MRSA pneumonia, mice with deletion of MLKL or mice treated with a RIPK1 inhibitor resulted in substantially increased lung inflammation, which was dependent on S. aureus pore forming toxins (α-toxin, LukAB or phenol soluble modulins [PSMs]) and enhanced IL-1β production (36). Although Q-VD-OPH did not inhibit necroptosis, these studies suggest that immunotherapeutics that target blocking necroptosis could reduce the bacterial burden and excessive inflammation to elicit a therapeutic benefit.

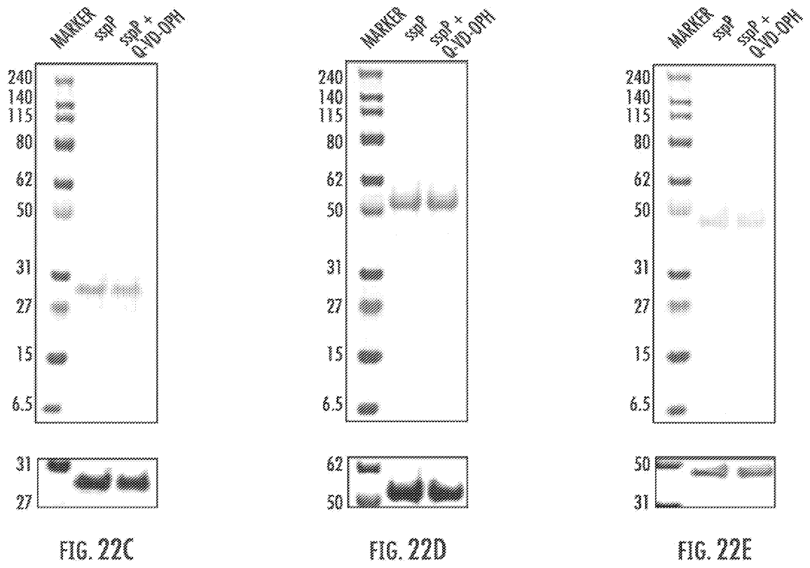

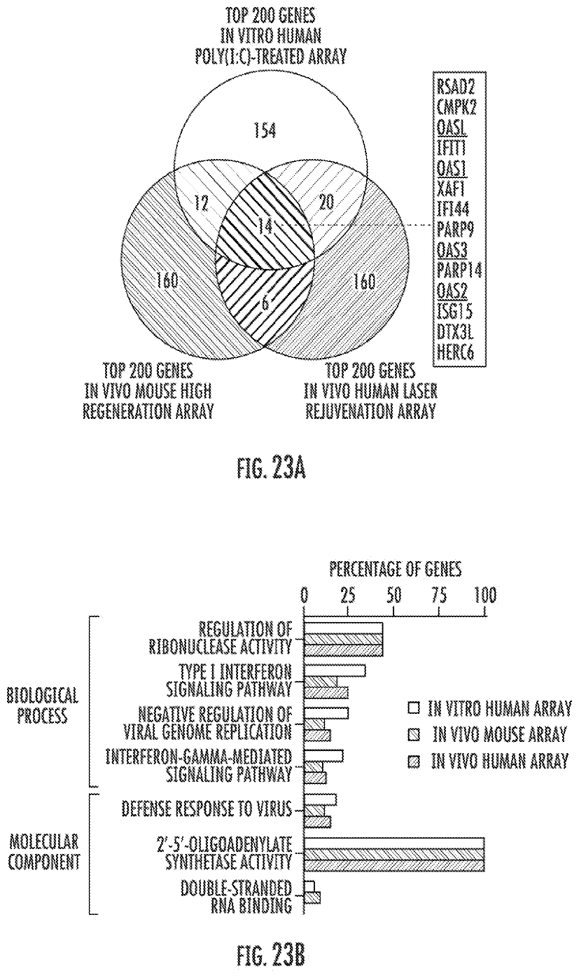

In addition to the effects of Q-VD-OPH on the host response, Q-VD-OPH could potentially inhibit bacterial virulence mechanisms. Specifically, we evaluated sspP and sspB produced by S. aureus, which inactivate innate immune components such as neutrophil chemokine CXCR2, modulate biofilm formation and can cause apoptosis-like cell death in neutrophils and monocytes (49-51) and speB produced by S. pyogenes, which is important in multiple virulence mechanisms (including during skin infections) and can activate processing of pro-IL-1β to IL-1β (52, 53). We found that Q-VD-OPH did not affect the activity of sspP, sspP or speB on gelatinase activity or elastin proteolysis (FIG. 22A, 22B). Furthermore, VD-OFH did not induce any physical conformational changes or cause degradation of sspP, sspB or speB (FIG. 22C-22E). It is possible that other bacterial virulence factors that could have been affected by Q-VD-OPH but a comprehensive investigation into every bacterial mechanism is beyond the scope of this study. Nonetheless, it is much more likely that the effects of Q-VD-OPH were on the host response, which is exemplified by the finding that Q-VD-OPH lacked efficacy in TNF^{-/-} mice TNF/IL-1R^{-/-} mice and in anti-TNF antibody-treated WT mice.

There are some limitations. First, the efficacy of Q-VD-OPH was evaluated in mouse models of CA-MRSA, S. pyogenes and P. aeruginosa infection, and studies in larger animal models and humans are needed to verify these results. This is particularly important as certain cytolytic toxins involved in human S. aureus infections (such as PVL) have less involvement in mouse models (5). Second, although the mechanism of Q-VD-OPH was further evaluated against CA-MRSA skin infection, it is unclear whether similar mechanisms were involved at other anatomical sites of S. aureus and CA-MRSA infection (such as in sepsis or pneumonia) and in the mechanisms of the efficacy of Q-VD-OPH against S. pyogenes, P. aeruginosa and other bacterial pathogens. These further experiments will be the subject of our future investigations. Caspases and especially caspase-8 are important for T cell and B cell proliferation and function (54, 55). Although our focus was on the effect of Q-VD-OPH on innate neutrophil and monocyte/macrophage responses in this report, we will evaluate the potential effect of Q-VD-OPH on these adaptive immune cell responses in our future work. Finally, Q-VD-OPH has broad efficacy against multiple caspases and although we determine its efficacy likely involves inhibition of caspases 3, 8 and 9 involved in apoptosis (30) and caspases 1, 8 and 11 involved in TNF production (38), it could be that more specific caspase inhibitors than Q-VD-OPH might have a similar therapeutic effect, providing the potential for more targeted therapy.

In conclusion, pan-caspase inhibition represents a potential host-directed immunotherapy against CA-MRSA, S. pyogenes and P. aeruginosa skin infections, a primary mechanism of action involving the inhibition apoptosis that promoted the survival of neutrophils and monocytes while also enhancing TNF-mediated host defense responses. With the increasing epidemic of antibiotic-resistant bacterial infections that is spreading among the global human population, pan-caspase inhibition could represent a valuable non-antibiotic alternative approach to help treat these increasingly common and often severe infections.

### REFERENCES

1. D. M. P. De Oliveira, B. M. Forde, T. J. Kidd, P. N. A. Harris, M. A. Schembri, S. A. Beatson, D. L. Paterson, M. J. Walker, Antimicrobial Resistance in ESKAPE Pathogens. Clinical microbiology reviews 33, (2020).
2. C. Y. Chiang, I. Uzoma, R. T. Moore, M. Gilbert, A. J. Duplantier, R. G. Panchal, Mitigating the Impact of Antibacterial Drug Resistance through Host-Directed Therapies: Current Progress, Outlook, and Challenges. mBio 9, (2018).
3. S. Y. Tong, J. S. Davis, E. Eichenberger, T. L. Holland, V. G. Fowler, Jr., Staphylococcus aureus infections: epidemiology, pathophysiology, clinical manifestations, and management. Clinical microbiology reviews 28, 603-661 (2015).
4. K. S. Kaye, L. A. Petty, A. F. Shorr, M. D. Zilberberg, Current Epidemiology, Etiology, and Burden of Acute Skin Infections in the United States. Clinical infectious diseases: an official publication of the Infectious Diseases Society of America 68, S193-S199 (2019).
5. L. S. Miller, V. G. Fowler, S. K. Shukla, W. E. Rose, R. A. Proctor, Development of a vaccine against Staphylococcus aureus invasive infections: Evidence based on human immunity, genetics and bacterial evasion mechanisms. FEMS microbiology reviews 44, 123-153 (2020).
6. E. A. Ezekwe, Jr., C. Weng, J. A. Duncan, ADAM10 Cell Surface Expression but Not Activity Is Critical for Staphylococcus aureus alpha-Hemolysin-Mediated Activation of the NLRP3 Inflammasome in Human Monocytes. Toxins 8, 95 (2016).
7. D. Holzinger, L. Gieldon, V. Mysore, N. Nippe, D. J. Taxman, J. A. Duncan, P. M. Broglie, K. Marketon, J. Austermann, T. Vogl, D. Foell, S. Niemann, G. Peters, J. Roth, B. Loftier, Staphylococcus aureus Panton-Valentine leukocidin induces an inflammatory response in human phagocytes via the NLRP3 inflammasome. Journal of leukocyte biology 92, 1069-1081 (2012).
8. J. H. Melehani, D. B. James, A. L. DuMont, V. J. Tones, J. A. Duncan, Staphylococcus aureus Leukocidin AB (LukAB) Kills Human Monocytes via Host NLRP3 and ASC when Extracellular, but Not Intracellular. PLoS pathogens 11, e1004970 (2015).
9. J. S. Cho, Y. Guo, R. I. Ramos, F. Hebroni, S. B. Plaisier, C. Xuan, J. L. Granick, H. Matsushima, A. Takashima, Y. Iwakura, A. L. Cheung, G. Cheng, D. J. Lee, S. I. Simon, L. S. Miller, Neutrophil-derived IL-1beta is sufficient for abscess formation in immunity against Staphylococcus aureus in mice. PLoS pathogens 8, e1003047 (2012).
10. C. Kebaier, R. R. Chamberland, I. C. Allen, X. Gao, P. M. Broglie, J. D. Hall, C. Jania, C. M. Doerschuk, S. L. Tilley, J. A. Duncan, Staphylococcus aureus alpha-hemolysin mediates virulence in a murine model of severe pneumonia through activation of the NLRP3 inflammasome. The Journal of infectious diseases 205, 807-817 (2012).
11. X. Wang, W. J. Eagen, J. C. Lee, Orchestration of human macrophage NLRP3 inflammasome activation by Staphylococcus aureus extracellular vesicles. Proceedings of the National Academy of Sciences of the United States of America 117, 3174-3184 (2020).
12. C. Y. Chi, C. C. Lin, I. C. Liao, Y. C. Yao, F. C. Shen, C. C. Liu, C. F. Lin, Panton-Valentine leukocidin facilitates the escape of Staphylococcus aureus from human keratinocyte endosomes and induces apoptosis. The Journal of infectious diseases 209, 224-235 (2014).
13. A. L. Genestier, M. C. Michallet, G. Prevost, G. Bellot, L. Chalabreysse, S. Peyrol, F. Thivolet, J. Etienne, G. Lina, F. M. Vallette, F. Vandenesch, L. Genestier, Staphylococcus aureus Panton-Valentine leukocidin directly targets mitochondria and induces Bax-independent apoptosis of human neutrophils. The Journal of clinical investigation 115, 3117-3127 (2005).
14. V. Winstel, O. Schneewind, D. Missiakas, Staphylococcus aureus Exploits the Host Apoptotic Pathway To Persist during Infection. mBio 10, (2019).
15. K. Kitur, S. Wachtel, A. Brown, M. Wickersham, F. Paulino, H. F. Penaloza, G. Soong, S. Bueno, D. Parker, A. Prince, Necroptosis Promotes Staphylococcus aureus Clearance by Inhibiting Excessive Inflammatory Signaling. Cell reports 16, 2219-2230 (2016).
16. T. Wong Fok Lung, I. R. Monk, K. P. Acker, A. Mu, N. Wang, S. A. Riquelme, S. Pires, L. P. Noguera, F. Dach, S. J. Gabryszewski, B. P. Howden, A. Prince, Staphylococcus aureus small colony variants impair host immunity by activating host cell glycolysis and inducing necroptosis. Nature microbiology 5, 141-153 (2020).
17. T. M. Caserta, A. N. Smith, A. D. Gultice, M. A. Reedy, T. L. Brown, Q-VD-OPh, a broad spectrum caspase inhibitor with potent antiapoptotic properties. Apoptosis: an international journal on programmed cell death 8, 345-352 (2003).
18. C. L. Keoni, T. L. Brown, Inhibition of Apoptosis and Efficacy of Pan Caspase Inhibitor, Q-VD-OPh, in Models of Human Disease. Journal of cell death 8, 1-7 (2015).
19. J. S. Braun, K. Prass, U. Dirnagl, A. Meisel, C. Meisel, Protection from brain damage and bacterial infection in murine stroke by the novel caspase-inhibitor Q-VD-OPH. Experimental neurology 206, 183-191 (2007).
20. M. Laforge, R. Silvestre, V. Rodrigues, J. Garibal, L. Campillo-Gimenez, S. Mouhamad, V. Monceaux, M. C. Cumont, H. Rabezanahary, A. Pruvost, A. Cordeiro-da-Silva, B. Hurtrel, G. Silvestri, A. Senik, J. Estaquier, The anti-caspase inhibitor Q-VD-OPH prevents AIDS disease progression in SIV-infected rhesus macaques. The Journal of clinical investigation 128, 1627-1640 (2018).
21. E. J. Lim, K. El Khobar, R. Chin, L. Earnest-Silveira, P. W. Angus, C. T. Bock, U. Nachbur, J. Silke, J. Torresi, Hepatitis C virus-induced hepatocyte cell death and protection by inhibition of apoptosis. The Journal of general virology 95, 2204-2215 (2014).
22. X. Teng, W. Chen, Z. Liu, T. Feng, H. Li, S. Ding, Y. Chen, Y. Zhang, X. Tang, D. Geng, NLRP3 Inflammasome Is Involved in Q-VD-OPH Induced Necroptosis Following Cerebral Ischemia-Reperfusion Injury. Neurochemical research 43, 1200-1209 (2018).
23. S. S. Burgener, N. G. F. Leborgne, S. J. Snipas, G. S. Salvesen, P. I. Bird, C. Benarafa, Cathepsin G Inhibition by Serpinbl and Serpinb6 Prevents Programmed Necrosis in Neutrophils and Monocytes and Reduces GSDMD-Driven Inflammation. Cell reports 27, 3646-3656 e3645 (2019).
24. D. W. Lee, S. Faubel, C. L. Edelstein, A pan caspase inhibitor decreases caspase-1, IL-1alpha and IL-1beta, and protects against necrosis of cisplatin-treated freshly isolated proximal tubules. Renal failure 37, 144-150 (2015).
25. C. A. Dillen, B. L. Pinsker, A. I. Marusina, A. A. Merleev, O. N. Farber, H. Liu, N. K. Archer, D. B. Lee, Y. Wang, R. V. Ortines, S. K. Lee, M. C. Marchitto, S. S. Cai, A. G. Ashbaugh, L. S. May, S. M. Holland, A. F. Freeman, L. G. Miller, M. R. Yeaman, S. I. Simon, J. D. Milner, E. Maverakis, L. S. Miller, Clonally expanded gammadelta T cells protect against Staphylococcus aureus skin reinfection. The Journal of clinical investigation 128, 1026-1042 (2018).
26. M. C. Marchitto, C. A. Dillen, H. Liu, R. J. Miller, N. K. Archer, R. V. Ortines, M. P. Alphonse, A. I. Marusina, A. A. Merleev, Y. Wang, B. L. Pinsker, A. S. Byrd, I. D. Brown, A. Ravipati, E. Zhang, S. S. Cai, N. Limjunyawong, X. Dong, M. R. Yeaman, S. I. Simon, W. Shen, S. K. Durum, R. L. O'Brien, E. Maverakis, L. S. Miller, Clonal Vgamma6(+)Vdelta4(+) T cells promote IL-17-mediated immunity against Staphylococcus aureus skin infection. Proceedings of the National Academy of Sciences of the United States of America 116, 10917-10926 (2019).
27. Y. Guo, R. I. Ramos, J. S. Cho, N. P. Donegan, A. L. Cheung, L. S. Miller, In vivo bioluminescence imaging to evaluate systemic and topical antibiotics against community-acquired methicillin-resistant Staphylococcus aureus-infected skin wounds in mice. Antimicrobial agents and chemotherapy 57, 855-863 (2013).
28. T. Kielian, E. D. Bearden, A. C. Baldwin, N. Esen, IL-1 and TNF-alpha play a pivotal role in the host immune response in a mouse model of Staphylococcus aureus-induced experimental brain abscess. Journal of neuropathology and experimental neurology 63, 381-396 (2004).
29. F. Hoss, V. Rolfes, M. R. Davanso, T. T. Braga, B. S. Franklin, Detection of ASC Speck Formation by Flow Cytometry and Chemical Cross-linking. Methods in molecular biology 1714, 149-165 (2018).
30. I. Jorgensen, M. Rayamajhi, E. A. Miao, Programmed cell death as a defence against infection. Nature reviews. Immunology 17, 151-164 (2017).
31. J. Knop, F. Hanses, T. Leist, N. M. Archin, S. Buchholz, J. Glasner, A. Gessner, A. K. Wege, Staphylococcus aureus Infection in Humanized Mice: A New Model to Study Pathogenicity Associated With Human Immune Response. The Journal of infectious diseases 212, 435-444 (2015).
32. S. D. Kobayashi, K. R. Braughton, A. M. Palazzolo-Ballance, A. D. Kennedy, E. Sampaio, E. Kristosturyan, A. R. Whitney, D. E. Sturdevant, D. W. Dorward, S. M. Holland, B. N. Kreiswirth, J. M. Musser, F. R. DeLeo, Rapid neutrophil destruction following phagocytosis of Staphylococcus aureus. Journal of innate immunity 2, 560-575 (2010).
33. P. K. Singh, A. Kumar, Mitochondria mediates caspase-dependent and independent retinal cell death in Staphylococcus aureus endophthalmitis. Cell death discovery 2, 16034 (2016).
34. J. H. Wang, Y. J. Zhou, P. He, Staphylococcus aureus induces apoptosis of human monocytic U937 cells via NF-kappaB signaling pathways. Microbial pathogenesis 49, 252-259 (2010).
35. M. C. Greenlee-Wacker, K. M. Rigby, S. D. Kobayashi, A. R. Porter, F. R. DeLeo, W. M. Nauseef, Phagocytosis of Staphylococcus aureus by human neutrophils prevents macrophage efferocytosis and induces programmed necrosis. Journal of immunology 192, 4709-4717 (2014).
36. K. Kitur, D. Parker, P. Nieto, D. S. Ahn, T. S. Cohen, S. Chung, S. Wachtel, S. Bueno, A. Prince, Toxin-induced necroptosis is a major mechanism of Staphylococcus aureus lung damage. PLoS pathogens 11, e1004820 (2015).
37. Y. Zhou, C. Niu, B. Ma, X. Xue, Z. Li, Z. Chen, F. Li, S. Zhou, X. Luo, Z. Hou, Inhibiting PSMalpha-induced neutrophil necroptosis protects mice with MRSA pneumonia by blocking the agr system. Cell death & disease 9, 362 (2018).
38. M. Fritsch, S. D. Gunther, R. Schwarzer, M. C. Albert, F. Schorn, J. P. Werthenbach, L. M. Schiffmann, N. Stair, H. Stocks, J. M. Seeger, M. Lamkanfi, M. Kronke, M. Pasparakis, H. Kashkar, Caspase-8 is the molecular switch for apoptosis, necroptosis and pyroptosis. Nature 575, 683-687 (2019).
39. R. K. S. Malireddi, P. Gurung, S. Kesavardhana, P. Samir, A. Burton, H. Mummareddy, P. Vogel, S. Pelletier, S. Burgula, T. D. Kanneganti, Innate immune priming in the absence of TAK1 drives RIPK1 kinase activity-independent pyroptosis, apoptosis, necroptosis, and inflammatory disease. The Journal of experimental medicine 217, (2020).
40. P. Orning, D. Weng, K. Starheim, D. Ratner, Z. Best, B. Lee, A. Brooks, S. Xia, H. Wu, M. A. Kelliher, S. B. Berger, P. J. Gough, J. Bertin, M. M. Proulx, J. D. Goguen, N. Kayagaki, K. A. Fitzgerald, E. Lien, Pathogen blockade of TAK1 triggers caspase-8-dependent cleavage of gasdermin D and cell death. Science 362, 1064-1069 (2018).
41. R. W. t. Davis, H. Eggleston, F. Johnson, M. Nahrendorf, P. E. Bock, T. Peterson, P. Panizzi, In Vivo Tracking of Streptococcal Infections of Subcutaneous Origin in a Murine Model. Molecular imaging and biology 17, 793-801 (2015).
42. J. M. Thompson, R. J. Miller, A. G. Ashbaugh, C. A. Dillen, J. E. Pickett, Y. Wang, R. V. Ortines, R. S. Sterling, K. P. Francis, N. M. Bernthal, T. S. Cohen, C. Tkaczyk, L. Yu, C. K. Stover, A. DiGiandomenico, B. R. Sellman, D. L. Thorek, L. S. Miller, Mouse model of Gram-negative prosthetic joint infection reveals therapeutic targets. JCI insight 3, (2018).
43. G. P. McStay, G. S. Salvesen, D. R. Green, Overlapping cleavage motif selectivity of caspases: implications for analysis of apoptotic pathways. Cell death and differentiation 15, 322-331 (2008).
44. M. Doerflinger, Y. Deng, P. Whitney, R. Salvamoser, S. Engel, A. J. Kueh, L. Tai, A. Bachem, E. Gressier, N. D. Geoghegan, S. Wilcox, K. L. Rogers, A. L. Garnham, M. A. Dengler, S. M. Bader, G. Ebert, J. S. Pearson, D. De Nardo, N. Wang, C. Yang, M. Pereira, C. E. Bryant, R. A. Strugnell, J. E. Vince, M. Pellegrini, A. Strasser, S. Bedoui, M. J. Herold, Flexible Usage and Interconnectivity of Diverse Cell Death Pathways Protect against Intracellular Infection. Immunity 53, 533-547 e537 (2020).
45. G. Brumatti, C. Ma, N. Lalaoui, N. Y. Nguyen, M. Navarro, M. C. Tanzer, J. Richmond, M. Ghisi, J. M. Salmon, N. Silke, G. Pomilio, S. P. Glaser, E. de Valle, R. Gugasyan, M. A. Gurthridge, S. M. Condon, R. W. Johnstone, R. Lock, G. Salvesen, A. Wei, D. L. Vaux, P. G. Ekert, J. Silke, The caspase-8 inhibitor emricasan combines with the SMAC mimetic birinapant to induce necroptosis and treat acute myeloid leukemia. Science translational medicine 8, 339ra369 (2016).
46. V. K. Viswanathan, A. Weflen, A. Koutsouris, J. L. Roxas, G. Hecht, Enteropathogenic E. coli-induced barrier function alteration is not a consequence of host cell apoptosis. American journal of physiology. Gastrointestinal and liver physiology 294, G1165-1170 (2008).
47. E. Butkevych, F. D. Lobo de Sa, P. K. Nattramilarasu, R. Bucker, Contribution of Epithelial Apoptosis and Subepithelial Immune Responses in Campylobacter jejuni-Induced Barrier Disruption. Frontiers in microbiology 11, 344 (2020).
48. O. Hultgren, H. P. Eugster, J. D. Sedgwick, H. Korner, A. Tarkowski, TNF/lymphotoxin-alpha double-mutant mice resist septic arthritis but display increased mortality in response to Staphylococcus aureus. Journal of immunology 161, 5937-5942 (1998).
49. A. J. Laarman, G. Mijnheer, J. M. Mootz, W. J. van Rooijen, M. Ruyken, C. L. Malone, E. C. Heezius, R. Ward, G. Milligan, J. A. van Strijp, C. J. de Haas, A. R. Horswill, K. P. van Kessel, S. H. Rooijakkers, Staphylococcus aureus Staphopain A inhibits CXCR2-dependent neutrophil activation and chemotaxis. The EMBO journal 31, 3607-3619 (2012).
50. J. M. Mootz, C. L. Malone, L. N. Shaw, A. R. Horswill, Staphopains modulate Staphylococcus aureus biofilm integrity. Infection and immunity 81, 3227-3238 (2013).
51. J. Smagur, K. Guzik, L. Magiera, M. Bzowska, M. Gruca, I. B. Thogersen, J. J. Enghild, J. Potempa, A new pathway of staphylococcal pathogenesis: apoptosis-like death induced by Staphopain B in human neutrophils and monocytes. Journal of innate immunity 1, 98-108 (2009).
52. C. N. LaRock, J. Todd, D. L. LaRock, J. Olson, A. J. O'Donoghue, A. A. Robertson, M. A. Cooper, H. M. Hoffman, V. Nizet, IL-1beta is an innate immune sensor of microbial proteolysis. Science immunology 1, (2016).
53. M. Saouda, W. Wu, P. Conran, M. D. Boyle, Streptococcal pyrogenic exotoxin B enhances tissue damage initiated by other Streptococcus pyogenes products. The Journal of infectious diseases 184, 723-731 (2001).
54. J. D. Graves, A. Craxton, E. A. Clark, Modulation and function of caspase pathways in B lymphocytes. Immunological reviews 197, 129-146 (2004).
55. S. Lakhani, R. A. Flavell, Caspases and T lymphocytes: a flip of the coin? Immunological reviews 193, 22-30 (2003).

EXAMPLE 2: The Viral Defense Gene RNase L Acts as a Regeneration Repressor. Mammalian injury responses are characterized by fibrosis and scarring rather than functional regeneration. Limited regenerative capacity in mammals could reflect a loss of pro-regeneration programs or active suppression by genes functioning akin to tumor suppressors. To uncover programs governing regeneration in mammals, we performed comprehensive transcript screening in human subjects after laser rejuvenation treatment and cross-referenced these transcripts to those found in mice with enhanced Wound Induced Hair Neogenesis (WIHN), a rare example of mammalian organogenesis^{1,2}. We find the anti-viral endoribonuclease RNase L to be a powerful suppressor of regeneration. RNasel^{-/-} mice exhibit remarkable regenerative capacity, with elevated WIHN through enhanced IL-36α. Consistent with the known role of RNase L to stimulate caspase-1, we find that pharmacologic inhibition of caspases promotes regeneration in a novel IL-36-dependent manner. Additionally, these responses are not limited to skin but extend to other organs, such as the colon, suggesting that suppression of regeneration is a fundamental characteristic of epithelial wound healing. Taken together, this work suggests that RNase L functions as a regeneration repressor gene in a functional tradeoff that prioritizes host antiviral abilities and is a target to enhance healing in multiple epithelial organs, perhaps even during viral infection.

### Materials and Methods

Mouse Lines. All wild-type and control mice used for in vivo experiments were on the C57BL/6J background. All mice were age-matched and co-housed until 6-weeks of age. Rnasel knockout mice (Rnasel^{tm1Slvm})³⁵ from the Silverman (co-author R. H. S.) lab were previously backcrossed on a C57BL/6 background. The Il36r knockout mice (Il1rl2^{tma1Hblu}) were acquired through a material transfer agreement between Johns Hopkins University School of Medicine and Amgen, Inc. Rnasel and Il36r double-knockout mice were generated by crossing both strains until viable homozygous mice were produced. All transgenic variants used were genotyped to confirm transgenic status using corresponding primers (Table 1). All mice were bred and housed at an American Association for the Accreditation of Laboratory Animal Care (AAALAC)-compliant facility and all experimental procedures were reviewed and approved by the Johns Hopkins University Institutional Animal Care and Use Committee (IACUC).

Wound Induced Hair Neogenesis (WIHN) Assay. All in vivo experimental surgical procedures were performed as previously characterized^{1,2,6-8}. In short, after exposure to vaporizing anesthesia (Baxter, Isoflurane) the dorsal side of male and female 3-week-old (21 days) mice were shaved at approximately 8-10 g of weight. Using surgical scissors, approximately 1.26 cm²×1.25 cm² of skin was excised creating wounds deep into the fascia. At 3 days post-wounding, a single dose (50 μl) of 100 μg/mL high molecular weight (HMW) polyinosinic-polycytidylic acid (Poly (I:C)) (Invivogen, tlrl-pic) was administered underneath the scab site via injection in both wild-type and transgenic mice as previously described^{2,8}. For functional experiments characterizing IL-36 on WIHN, a single dose (50 μl) of 1 μg/mL recombinant mouse IL-36α protein (R&D Systems, 7059-ML/CF) was injected underneath the scab site at 7 days post-wounding. For rescue experiments using the small molecule, broad spectrum pan-caspase inhibitor, 20 ul of 5 mg/mL Q-VD-OPh (in DMSO) was diluted 1304 of phosphate-buffered saline (PBS) (1.33 mM final concentration) and injected intraperitoneally in mice 24 hours prior to wounding and approximately 10 days after wounding (scab detachment). Approximately after 3-weeks post wounding (˜21 days), neogenic hair follicles in the re-epithelialized skin tissue were quantified using reflectance confocal scanning laser microscopy (CSLM) as previously demonstrated^{2,8}.

Human and Mouse Keratinocyte Isolation and Culture. Primary human keratinocytes were isolated from fresh neonatal foreskins stored in CO₂-independent medium (Gibco, 18045088) as previously described^{2,8}, adhering to Johns Hopkins University IRBs (NA 0033375, NA 00075350, IRB00028768). After removing subcutaneous fat, foreskin tissue was enzymatically digested overnight at 4° C. in a 0.4% dispase II solution (Sigma, D4693). After tissue disaggregation, the epidermis was incubated in either 0.025% trypsin/EDTA (Lonza, CC-5012) at 37° C. or accutase (CELLnTEC, CnT-Accutase-100) at room temperature (˜23° C.). The epidermal sheets were passed through a cell strainer to obtain keratinocytes that were cultured in keratinocyte growth medium (KGM-Gold) supplemented with necessary growth factors and antibiotics (Lonza, 192060). Primary mouse keratinocytes were isolated from tails as described above, with the addition of a 10 uM rho-kinase inhibitor (Y-27632, Cayman Chemical, 10005583) in KGM-Gold media as demonstrated previously³⁶. All human and mouse keratinocytes were passaged at least once prior to use in all in vitro experiments.

In vitro Supernatant Protein Concentration. To measure secreted IL-36 protein isoforms media supernatant from cultured mouse or human keratinocytes was concentrated using an Amicon Ultra centrifugal filter device with a nominal molecular weight limit (NMWL) of 3 kDa (MilliporeSigma, UFC200324). All samples were concentrated ˜20× prior to use for downstream applications (i.e., immunoblotting).

RNA Isolation and Quality Analysis. Total RNA (including small non-coding RNA) from tissue was isolated using a TRIzol-based, non-phase separation spin column purification method (Zymo Research, R2073). Total RNA from human keratinocytes was purified using the RNeasy Mini Kit (Qiagen, 74106). In both instances, RNA was incubated with DNase I to efficiently digest DNA prior to elution. RNA purity and quantity was calculated using a UV-Vis spectrophotometer (NanoDrop2000c (Thermo Fisher Scientific, ND-2000c). Total RNA quality was assessed by measuring 28S/18S ribosomal RNA ratios and scoring RNA Quality/Integrity Number (RQN/RIN) values via capillary electrophoresis using either 2100 Bioanalyzer (Agilent, Santa Clara, Calif.) or Fragment Analyzer CE (Agilent, Santa Clara, Calif.).

Quantitative real-time PCR (qRT-PCR). A high-capacity reverse transcription kit (Applied Biosystems, 4368813) was used to synthesize cDNA from mRNA. TaqMan probes against target genes of interest were designed using fluorescein (FAM) dyes. Probes against housekeeping genes RPLP0 and β-actin were used for human and mouse-derived cDNA respectively. Relative gene expression was determined using the comparative (ΔΔCₜ) method, derived from cycling threshold (Ct) differences from target and housekeeping genes.

Flow Cytometry. Cell suspensions were prepared by digesting mouse skin tissue in a cocktail consisting of Liberase TL (Roche, 5401020001), DNase I (Sigma, DN25) and antibiotics (Invitrogen, 15140) in RPMI 1640 (Gibco, 11875093). For FACS, cells were washed and first viability stained using the Zombie Aqua dye (BioLegend, 423101). After blocking, cells were stained with an antibody cocktail (Extended Table 2) and BD Horizon Brilliant Violet (BV) buffer (BD, 563794) to minimize non-specific spectral overlap of similar conjugates. Finally, cells were resuspended in BD stabilizing fixative prior to FACS. All flow cytometry experiments were performed on a BD LSR II and downstream analysis of data was performed using Cytobank.

In vivo microarray. Total RNA was isolated from mouse tissue at the time of skin re-epithelialization and scab detachment from the wound (˜10 days post-wounding) from both wild-type and Rnasel^{-/-} mice. RNA was submitted to the MIMI Deep Sequencing & Microarray core facility and profiled using the Affymetrix Clariom™ S mouse array platform according to the manufacturer's protocols. Gene chips were scanned generating CEL pixel intensity files, which were processed and analyzed using Partek® Genomics Suite™ software and Robust Multichip Analysis (RMA) algorithm was used for normalization.

In vivo circRNA-sequencing. 1 cm² skin tissue biopsies were taken from both wild-type and Rnasel^{-/-} mice and submitted to CD Genomics (Shirley, N.Y.) for sequencing. Briefly, after total RNA isolation, samples were treated with RNase R to digest linear RNA. Following strand-specific library preparation, samples were sequenced on a HiSeq PE150 (Illumina, San Diego, Calif.). We calculated differential expression and enrichment, for functional analyses. Differential expression analysis was performed using the generalized fold change (GFOLD) algorithm and genes meeting the 0.5 threshold were ranked. The data was aligned to the GRCm38.p6 reference genome.

In vitro RNA-sequencing. Total RNA (including small RNAs) from primary human keratinocytes treated with non-targeting and RNase L-targeting siRNA in the presence or absence of 10 μg/mL poly(I:C) were submitted to the Experimental and Computational Genomics Core (ECGC) at the Sidney Kimmel Comprehensive Cancer Center (SKCCC) for RNA sequencing. Libraries were prepared using the TruSeq Stranded Total RNA LT Sample Prep Kit (Illumina, 15031048) for polyadenylated RNA selection, followed by barcoding. Sequencing was performed on the HiSeq2500 platform (Illumina, San Diego, Calif.), producing 50 million 100×100-bp paired-end reads. Illumina's CASAVA 1.8.4 was used to convert BCL files to FASTQ files using default parameters. RSEM-1.3.0's EBSeq was used for differential expression analysis and for running the alignments as well as generating gene and transcript expression levels. The data was aligned to the GRCh38 reference genome using the Spliced Transcripts Alignment to a Reference (STAR) method. Uploaded data can be found at NIH GEO GSE164667.

Histology. Biopsies from mouse colon and skin tissue were removed and fixed in 4% paraformaldehyde overnight and then transferred to 70% ethanol. Samples were then submitted to the Johns Hopkins Oncology Tissue Services Core facility where they were embedded in paraffin. Tissue sections were obtained at 4 μm thickness and mounted onto glass slides, followed by hematoxylin and eosin (H&E) staining.

Immunofluorescence, immunocytochemistry, and immunohistochemistry. Immunofluorescence and fluorescence microscopy for mouse tissue was performed on de-paraffinized sections following heat-induced antigen retrieval using Target Retrieval Solution (Agilent Dako, 5169984-2). After washing and permeabilization in TBS-T universal buffer (0.2% Triton X-100 in tris-buffered saline), sections were blocked at room-temperature in 5% goat, donkey or fetal bovine serum with 1% bovine serum albumin. Tissue sections were then incubated overnight at 4° C. with primary antibodies at the suggested concentrations (Extended Table 3) in Antibody Diluent (Agilent Dako, S080983-2). Following subsequent washing, sections were incubated in fluorescent-dye conjugated secondary antibodies diluted in antibody diluent for 1 hour at room temperature. After final washing, sections were mounted with VECTASHIELD® Hardset™ Antifade Mounting Medium with DAPI (Vector Laboratories, H-1500) for nuclear staining. Human keratinocytes were prepared similarly, with the exception of antigen retrieval. All slides were imaged using either the DFC365FX (Leica) or Eclipse E-800 (Nikon) at 10×, 20×, and 40× magnifications.

Colitis Model. In order to induce colitis, adult wild-type, Rnasel^{-/-}, and 1136r^{-/-} mice were fed up to 4% (w/v) 36-50 kDa dextran sodium sulfate (DSS) (MP Biomedicals, 160110) in sterile water³⁷. Weight changes were monitored and recorded every day. For rescue experiments using the pan-caspase inhibitor Q-VD-OPh, mice were treated at the same concentrations as in WIHN experiments and were injected intraperitoneally during the day when noticeable weight loss began to occur (˜day 3-4). Mice were eventually sacrificed and colon lengths were extracted for gross examination and downstream FACS and protein analysis.

Immunoblot analysis. Human keratinocytes and mouse tissue were resuspended and lysed in M-PER Mammalian Protein Extraction Reagent (Thermo Fisher Scientific, 78501) containing an EDTA-based, broad-spectrum protease inhibitor cocktail (Thermo Fisher Scientific, 87786). Samples were then ultrasonically disrupted using a probe and then protein concentrations were quantified using the colorimetric BCA assay (Thermo Fisher Scientific, 23225). Proteins and ladder were loaded on to a denaturing NuPAGE Bis-Tris gel at a 4-12% gradient (Thermo Fisher Scientific, NP0321BOX) followed by electrophoresis. Proteins were then transferred and bound to a methanol-activated PVDF membrane (Bio-Rad, Hercules, Calif.). After a brief wash with 0.1% Tween-20 buffer, membranes were incubated in 5% non-fat dry milk (NFDM) blocking buffer for 1 hour at room temperature followed by an overnight incubation with primary antibody in blocking buffer at 4° C. Membranes were washed and incubated with corresponding horseradish peroxidase (HRP)-conjugated secondary antibodies at room temperature for 1 hour. All membranes were subsequently similarly probed for β-actin as a loading control for proteins. Proteins were detected on membranes using a luminol-based, HRP-reactive chemiluminescent substrate (Thermo Fisher Scientific, 34577) and visualized on a ChemiDoc XRS+ imaging system (Bio-Rad, Hercules, Calif.). Concentrated secreted protein from mouse keratinocytes were similarly processed.

siRNA transfection. For both mouse and human keratinocytes, cells were seeded at 50,000 cells/well (RNA) or 100,000 cells/well (protein) in 12-well or 6-well plates respectively. Non-targeting and gene-specific siRNAs (Dharmacon) (Extended Table 4) for human and mouse were used. Briefly, 25 nM of siRNA was pooled with Lipofectamine® RNAiMAX transfection reagent (Thermo Fisher Scientific, 13778150) in reduced serum OPTI-MEM media (Gibco, 31985062) and added to cultured keratinocytes for 48 hours to achieve maximum gene knockdown efficiency.

Proteomics analysis. Keratinocytes from wild-type and Rnasel^{-/-} mice were prepared for protein analysis. Briefly, after saline washing, samples were lysed in 5% sodium deoxycholate (DOC) detergent. After sequential peptide processing (reduction, alkylation and trypsinolyzation), downstream resolving and analysis were performed on a nanoACQUITY UPLC system with a Tribrid Orbitrap-quadrupole-linear ion trap mass spectrometer (Thermo Fisher). The UniProt mouse reference proteome was used to align tandem mass spectra (MS-MS) data in conjunction with the Sequest HT algorithm. Protein abundance ratios were calculated by comparing MS1 peptide ion intensity peaks. Machine-learning based software (Percolator) was used for peptide identification and validated at an FDR of at least 0.05. Center versus Edge proteomics is available at the data repository of University of Maryland metallotherapeutics research center, Baltimore and the mass spectrometry proteomics data have been deposited to the ProteomeXchange Consortium via the PRIDE partner repository with the dataset identifier PXD013854. The RNaseL siRNA proteome data set can be found at px-submission #PXD023375 in the ProteomeXchange Consortium.

Retinoid derivatives measurements. All in vivo skin samples from wild-type and Rnasel^{-/-} mice were collected and snap-frozen and stored at −80° C. Samples were then processed as described by Kim et al.⁸. Briefly, after homogenization, endogenous retinoids were extracted under low-intensity yellow light via dual-step liquid partitioning. Multistep partitioning was achieved using a highly selective liquid chromatography tandem mass spectrometry (LC-MS/MS) technique, LC-MRM³. Retinoic acid measurements were performed using a Shimadzu Prominence ultra-fast liquid chromatograph (UFLC_{XR}) (Shimadzu, Columbia, Md.) with AB Sciex 5500/6500+ hybrid triple quadrupole-linear ion trap (QqQ(LIT)) mass spectrometers (AB Sciex, Framingham, Mass.) using atmospheric pressure chemical ionization (APCI) conducted in pseudo-molecular MH+ mode. For all experiments, the reference controls used were 4,4-dimethyl-RA, retinyl acetate, and total retinyl ester for retinoic acid, retinol, and retinyl ester, respectively. Endogenous retinol and retinyl ester were measured and analyzed via UHPLC-UV using rapid resolution, reverse-phase Zorbax columns (SB-C18, Agilent) on a quaternary-based ACQUITY UPLC H-Class System (Waters Corporation, Milford, Mass.) with an ultraviolet detector.

RNaseL microarray analysis. Protein and gene annotation enrichment and ontology analyses were performed using the Database for Annotation, Visualization and Integrated Discovery (DAVID) and the PANTHER classification system. All gene list exploratory analyses were statistically significant using the Fisher's exact test, with the Benjamini-Hochberg false discovery rate (FDR) correction or Bonferroni correction. RNaseL null mouse microarrays are available in GSE164003 NCBI GEO.

Cross-array Analysis. GSE50418 NCBI GEO was used for outbread and inbread strain analysis; GSE131789 NCBI GEO was used for human laser array; GSE92646 NCBI GEO was used for Poly I:C treated keratinocytes. To avoid comparing directly the signal values between different microarray platforms, instead we compared their fold change results since the genes' expression fold change values themselves derive from comparing like (from the same array) values, which avoids any array-to-array idiosyncrasies. Each array experiment thus generated a ratio for each probe set of high regenerating sample over low regenerating sample. Also, defined orthologous genes allows expression results to be compared between different species and model organisms. Using these principles, we compared the previous results from human subjects, using the Affymetrix PrimeView array, with those from mice using the Affymetrix mouse MoEx-1_0-st-v1 and Mouse Clariom_S arrays⁸. The gene transcript annotation of each array was first updated to current HUGO/MGI/NCBI nomenclature after which the mouse genes were mapped to their human orthologs using the NCBI HomoloGene and SMI databases, along with the Human Gene Nomenclature Committee's HGNC Comparison of Orthology Predictions (HCOP) database³⁸. Among a standard gene set that was the identical for every experiment, the respective log₂ fold changes for each orthologous gene in each of the three biological comparisons were then rank ordered from those with the highest average regeneration ratio to lowest regeneration ratio. That rank order was averaged across the 3 experiments and again sorted from highest to lowest average.

Heatmap analysis. Gene expression was standardized for definition of differentially expressed genes, and GO and KEGG enrichment analysis. We used the R package limma to define DEGs among different groups³⁹. We used the clusterProfiler R package for gene-annotation enrichment analysis⁴⁰. Finally, the visualization of these results was created using the ggplots2 R package.

Quantification and Statistical Analysis. All in vivo and in vitro experiments were performed in at least individual instances. Univariate statistical analysis was performed using Student's t-test and multivariate analysis was performed using ANOVA. All statistical analyses and graphical representations were generated using GraphPad Prism software. Statistical significance is defined as p-values <0.05 derived from the standard error of mean calculations.

### Results

Upon amputation, animals such as urodele amphibians form a dedifferentiated cell cluster, known as the blastema, which coordinates whole appendage regeneration^{3,4}. Mammals by contrast lack this capacity for epimorphic regeneration. One exception to this is the induction of de novo hair follicles in adult skin after full-thickness excisional wounding in mice and rabbits, a process termed wound induced hair neogenesis (WIHN)^{1,5}. WIHN is characterized by a morphogenic cascade that recapitulates embryogenic events, whereby multipotent keratinocyte progenitors communicate with mesenchymal fibroblasts to form neogenic hair follicles and surrounding skin. Important advances have been made in defining the role of developmental pathways such as Wnt, Shh, and Fgf in driving WIHN^{1,6,7}. Among the earliest mediators activating these developmental pathways are non-coding RNAs, some of which include regions of double-stranded RNA (dsRNA) that are released as a Damage Associated Molecular Pattern (DAMP) during tissue injury^{2,8,9}. However, the downstream targets of these dsRNAs that promote regeneration are incompletely understood. Understanding the totality of these regeneration pathways will allow the development of active therapeutics to promote healing rather than the current medical standard of care: simple supportive measures after acute injury.

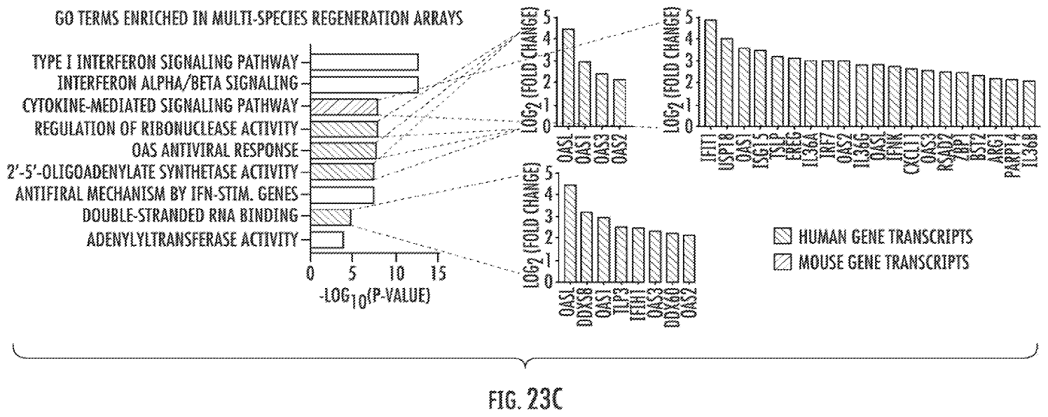

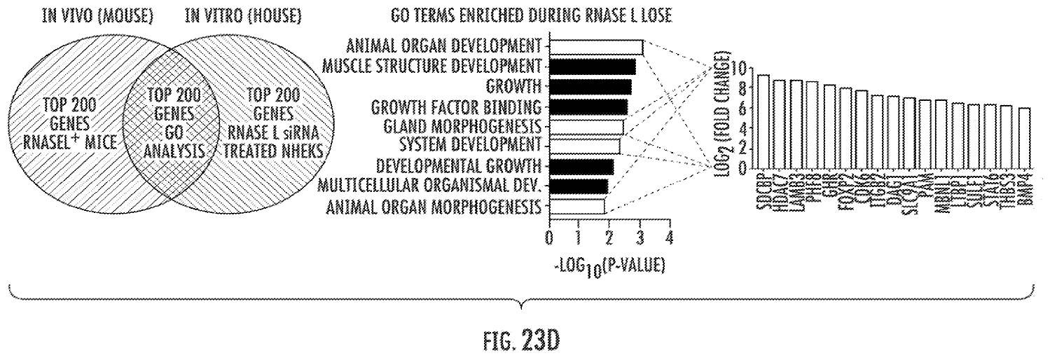

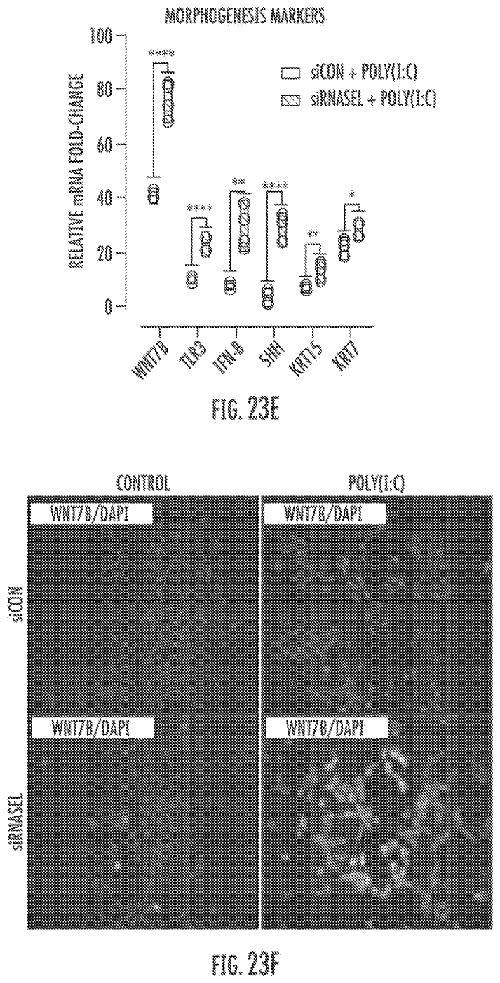

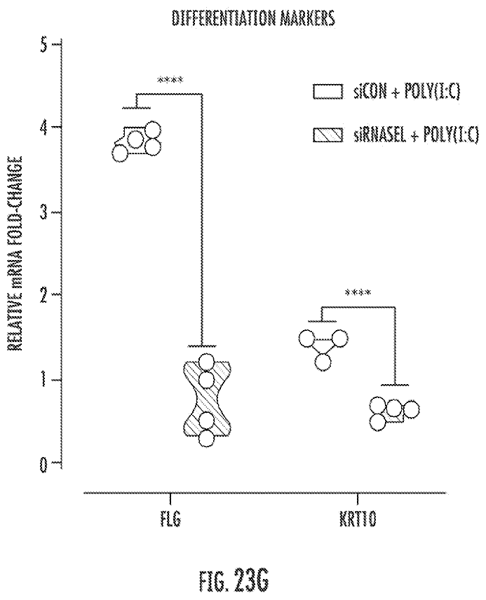

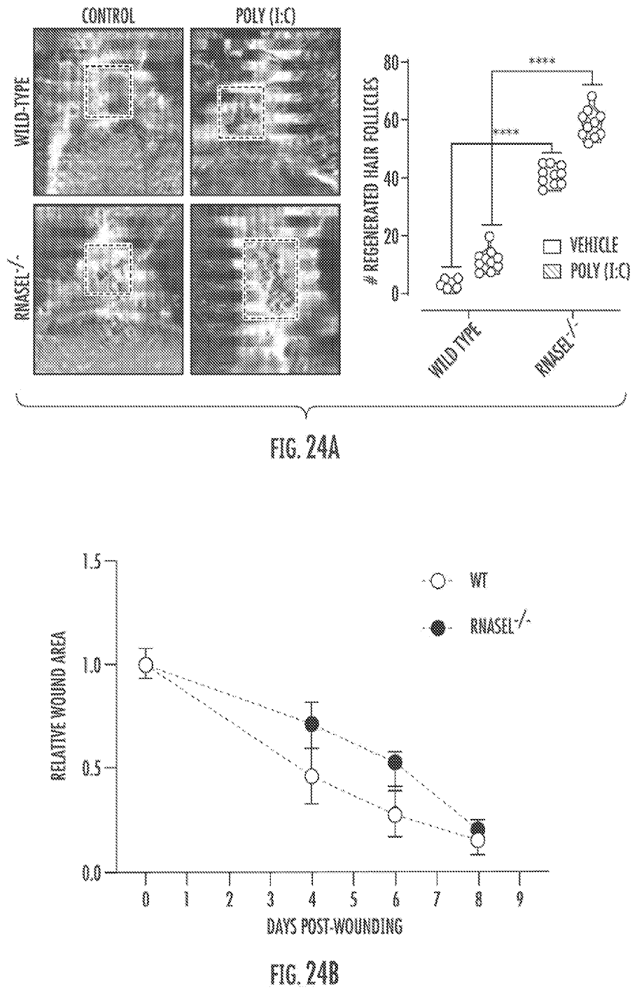

To define common mechanisms that dictate tissue regeneration, we performed transcriptome analysis of injury-mediated rejuvenation in humans and mice. We probed the intersection of 3 distinct transcriptome screens and defined the top 200 genes in: (1) wild-type outbred strains of mice with a high capacity for WIHN (C57BL/6×FVB×SJL); (2) human subjects after laser rejuvenation to ameliorate photoaging; and (3) cultured human keratinocytes treated with synthetic dsRNA poly(I):poly(C) (polyI:C)⁸ as a positive control. A dramatic overlap signature is observed, including all human members of a class of dsRNA sensors, the 2′-5′-oligoadenylate synthetase (OAS) gene family (OAS1, OAS2, OAS3, and OASL; FIG. 23A, which are known to be induced in response to polyI:C in an interferon response^{10,11}. Gene ontology analysis reveals that OAS RNA expression is predominantly found in all three datasets, both individually (FIG. 23B) and together (FIG. 23C). These results demonstrate a correlation between OAS expression and regeneration responses in mice and humans.

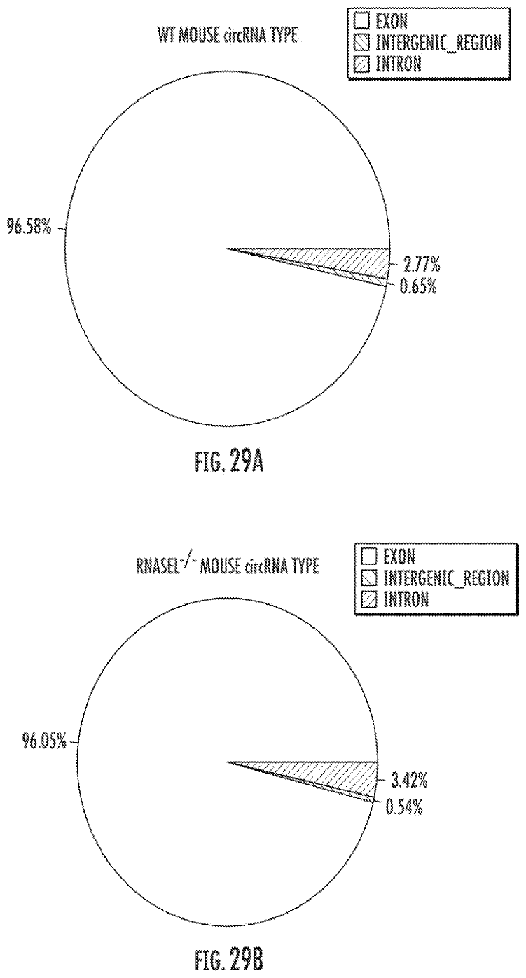

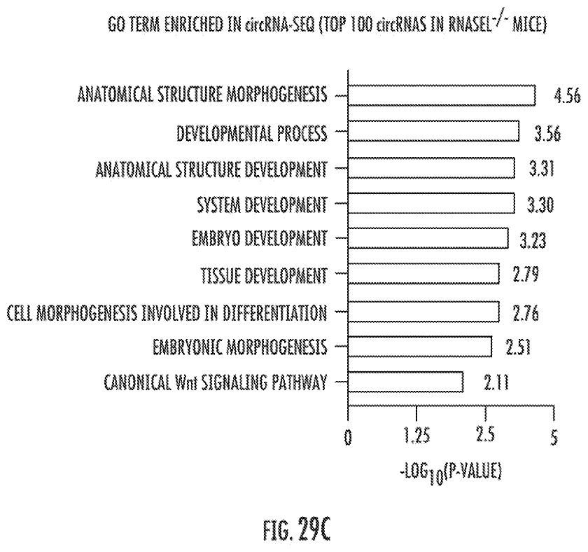

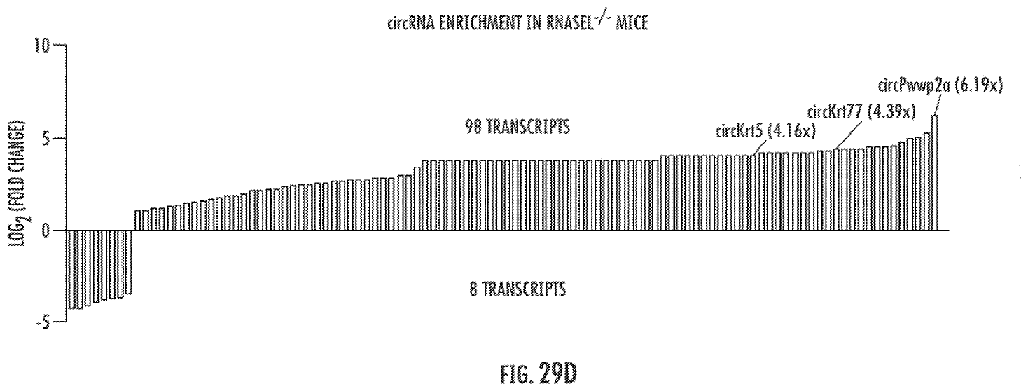

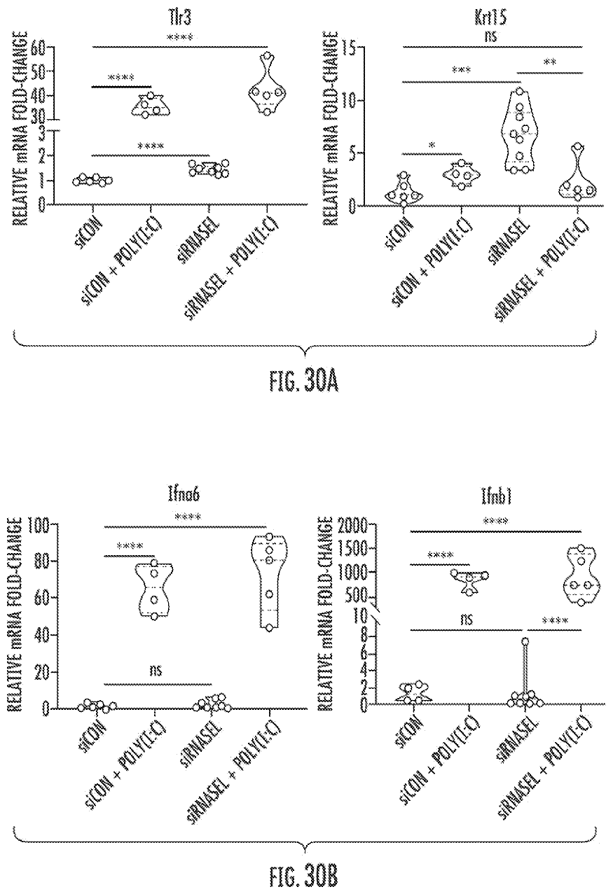

OASes are antiviral enzymes that produce 5′-triphosphorylated 2′-5′ adenylyl oligomers that in turn activate the endoribonuclease RNase L¹². RNase L has broad functions including antiviral immunity and recently defined circRNA metabolism^{13,14}. Given that RNase L represents the convergence point for multiple distinct OASes, we sought to identify the role of RNase L during regeneration. We thus first tested the effect of RNase L loss-of-function on gene expression in a mouse model of whole-body deletion and in vitro after gene silencing in human keratinocytes. Common pathways induced in both contexts of RNase L loss include both developmental and morphogenesis pathways (FIG. 23D), which are also seen in proteomics analysis (FIG. 28A-28B). We also find that circRNAs are more abundant in the skin of Rnasel^{-/-} mice compared to wild-type (WT) mice, corroborating previous findings (FIG. 29A-29B). In particular, circRNAs derived from genes that control stem cell fate and early development, such as PWWP Domain Containing 2A (circPwwp2a), are upregulated in Rnasel^{-/-} mice (FIG. 29C-29D). These results suggest that RNase L loss alters regenerative response pathways via associated changes in the circRNA repertoire, with implications for wound healing¹⁵.

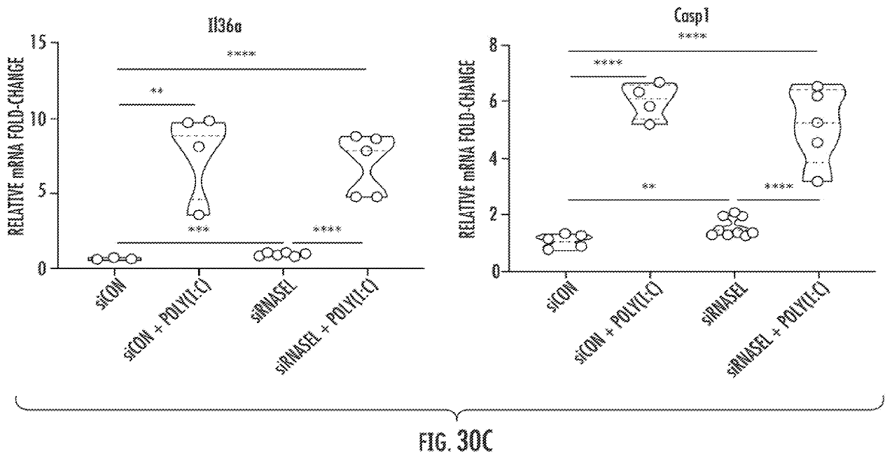

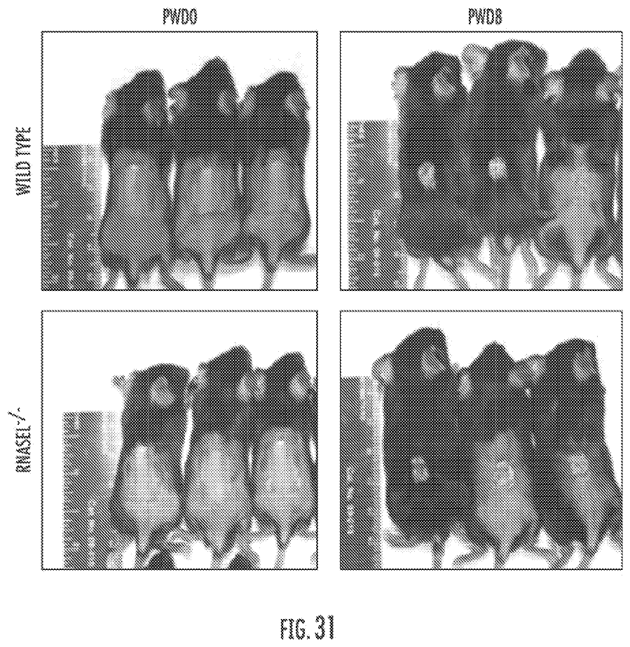

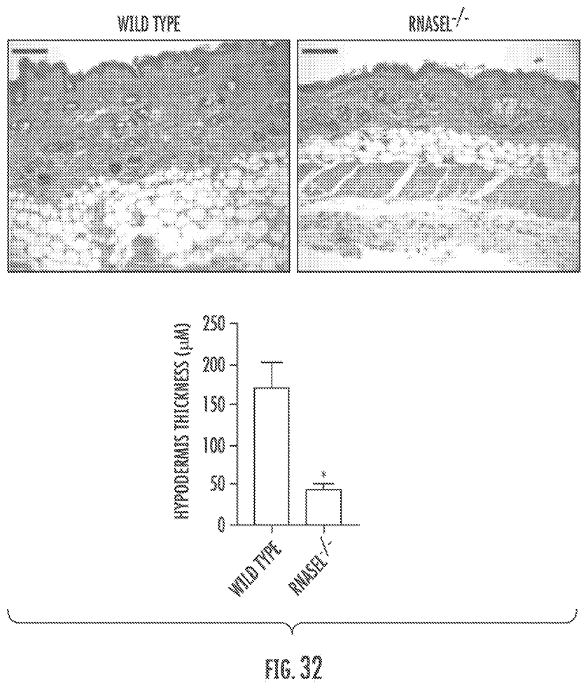

Double-stranded RNA is known to enhance regeneration by respectively inducing and inhibiting undifferentiated and differentiated cell states via Tlr3². We thus added the dsRNA polyI:C to normal adult human keratinocytes with or without RNase L depletion to characterize the polarity of RNase L function in the context of regeneration to either promote or suppress regeneration. We find that polyI:C treatment coupled with RNase L loss has an even greater effect than polyI:C addition alone to hyper-activate the expression of morphogenesis and stem cell transcripts, such as WNT7B, TLR3 (toll-like receptor 3), SHH (sonic hedgehog), KRT15, and KRT7 (FIG. 23E-23F). In parallel, differentiation-associated transcripts are down regulated, including FLG (filaggrin) and KRT10 (FIG. 23G). The same is seen in mouse keratinocytes (FIG. 30A). Also, despite reports of non-specific or cell-type-specific IFN activation, we find no discernable differences in IFN mRNA after RNase L depletion itself (FIG. 30B)^{11,16}. Because of the known dsRNA-OAS-RNase L activation cascade, these results suggest that RNase L might be a regeneration repressor gene whose loss of function increases regeneration after injury.

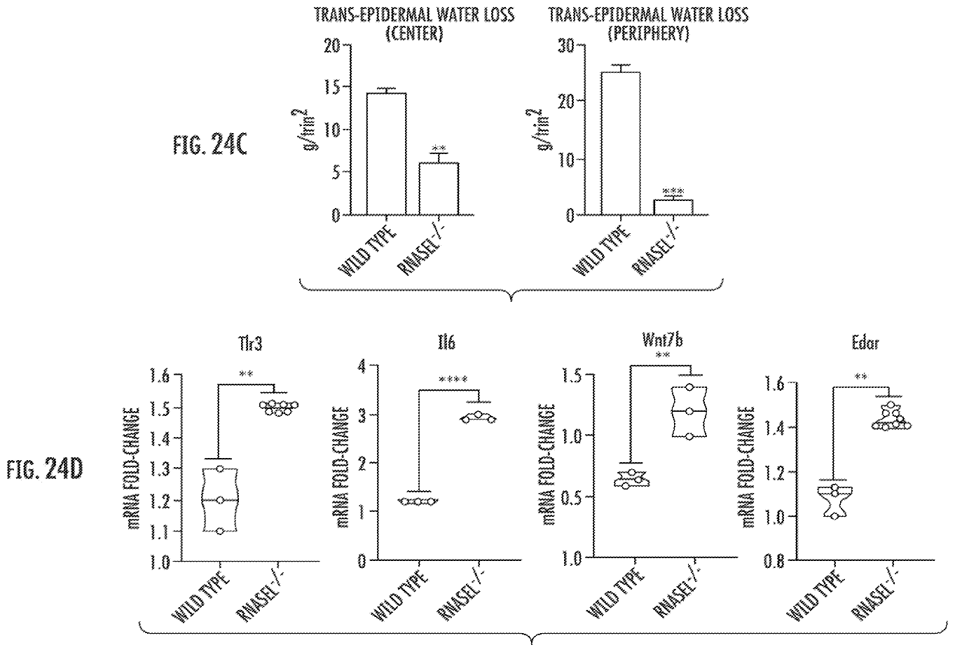

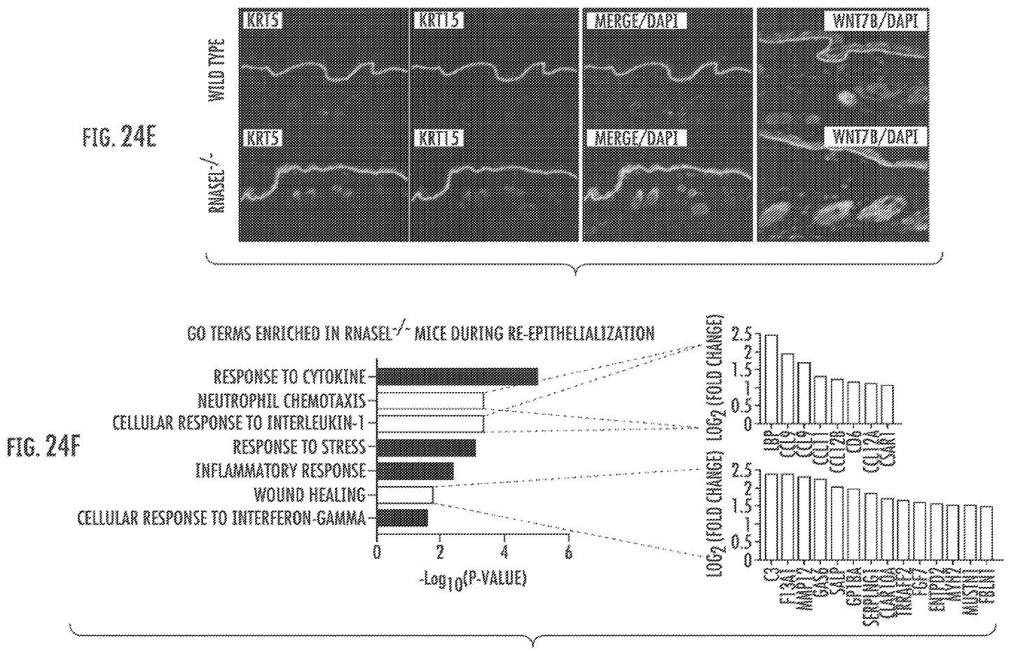

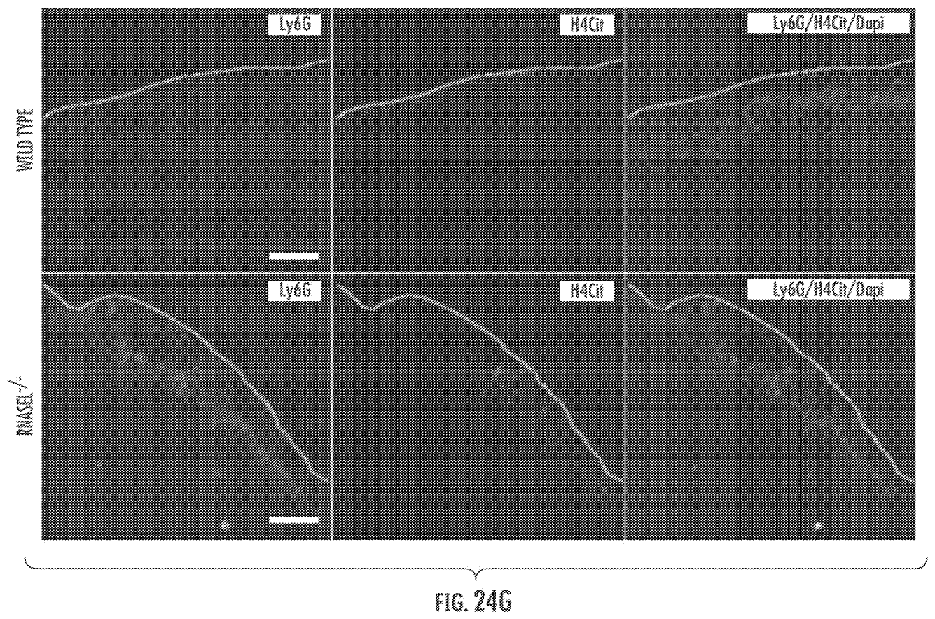

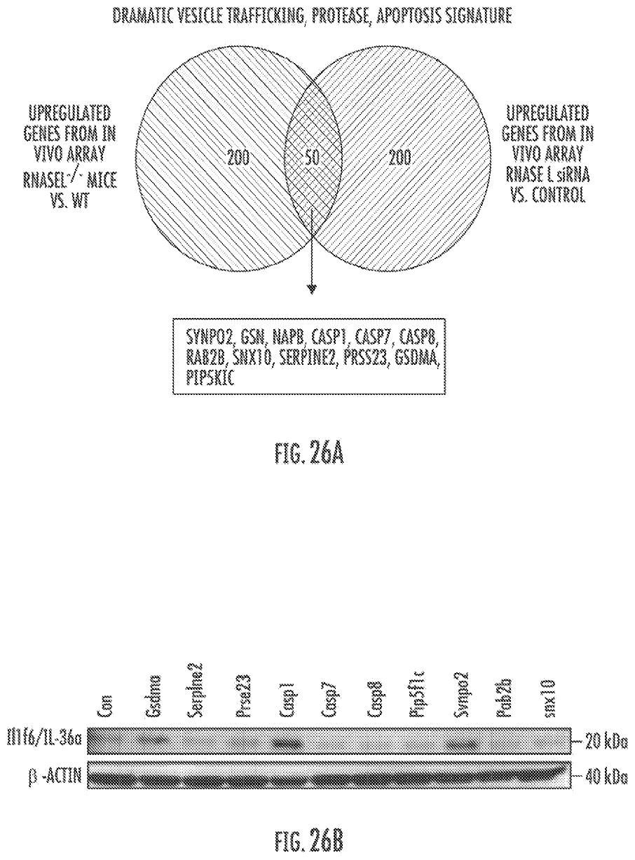

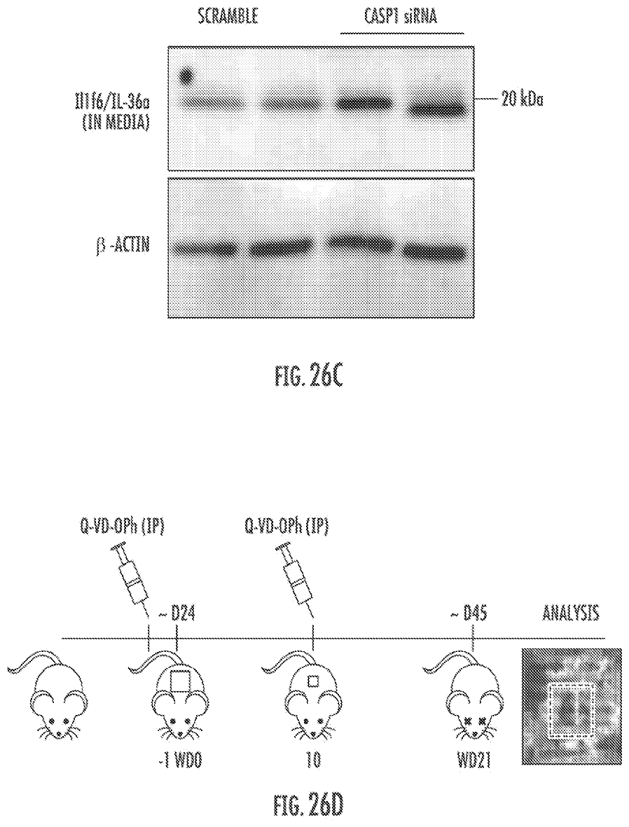

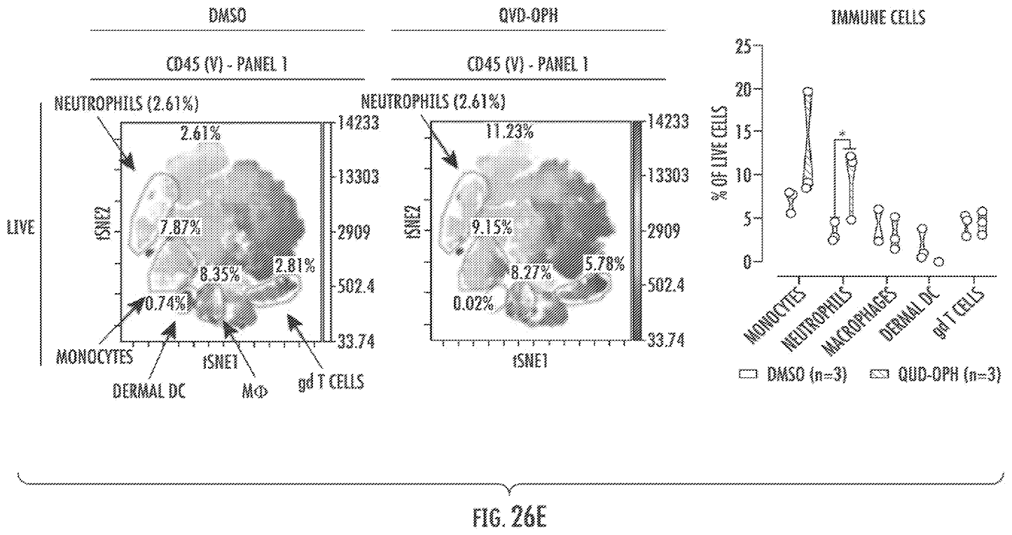

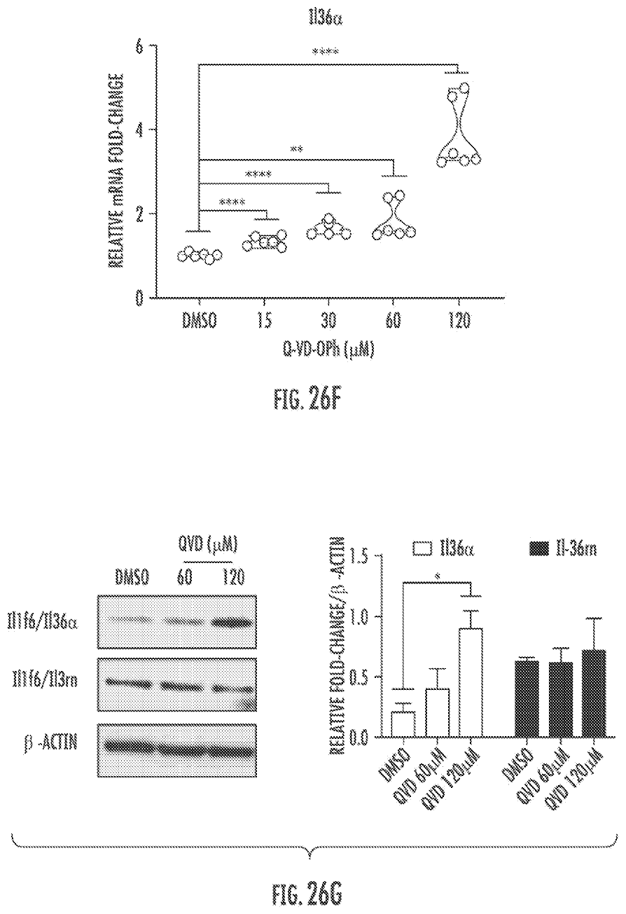

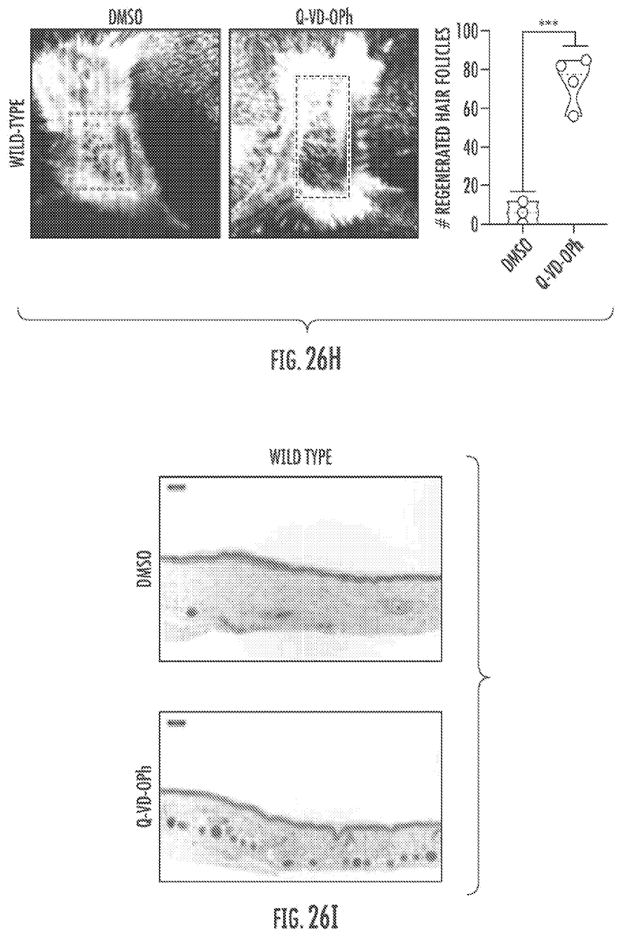

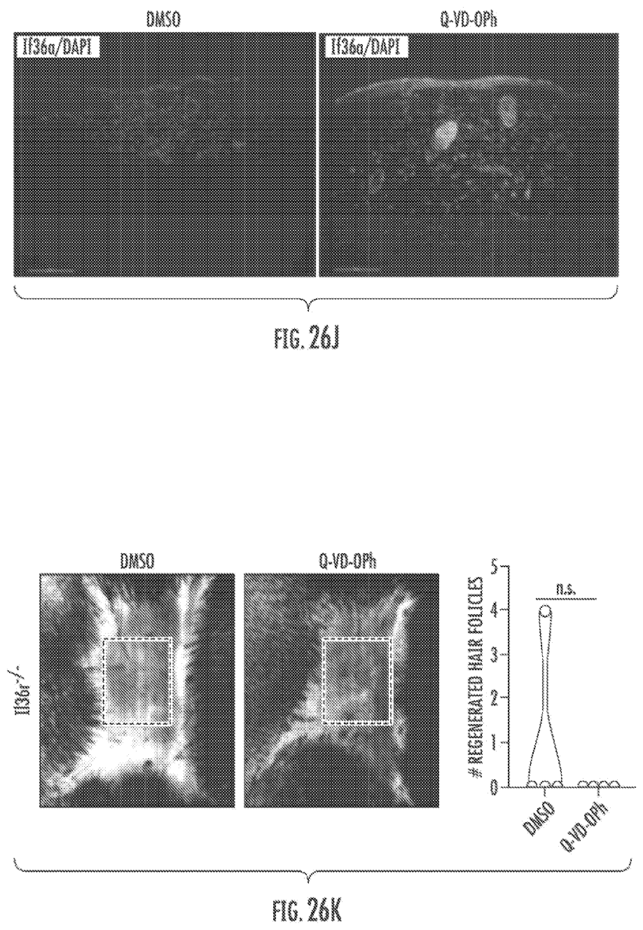

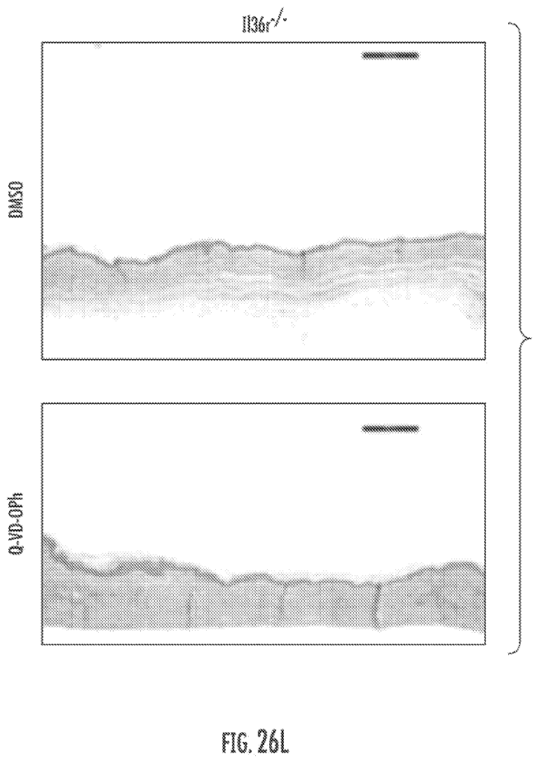

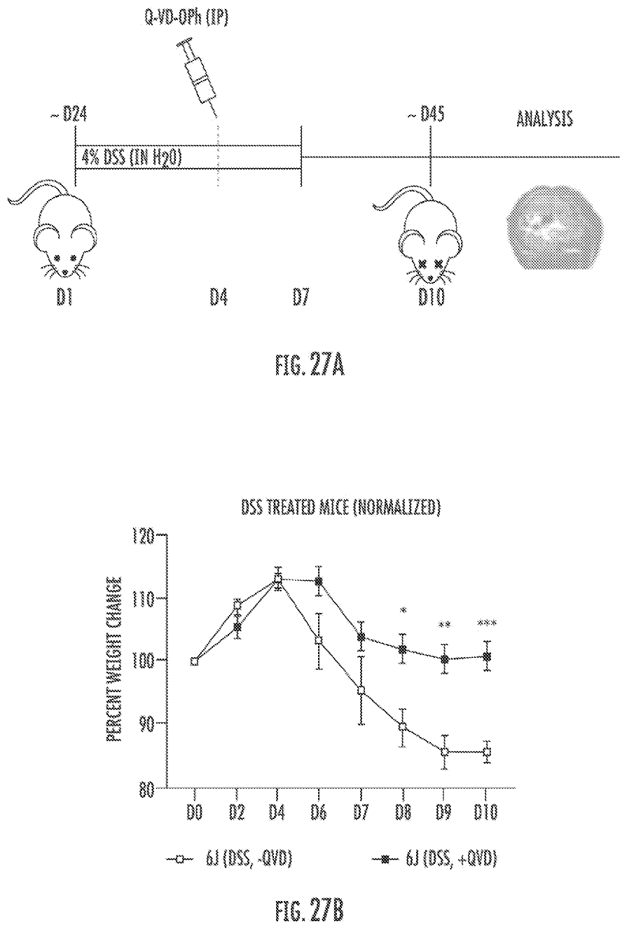

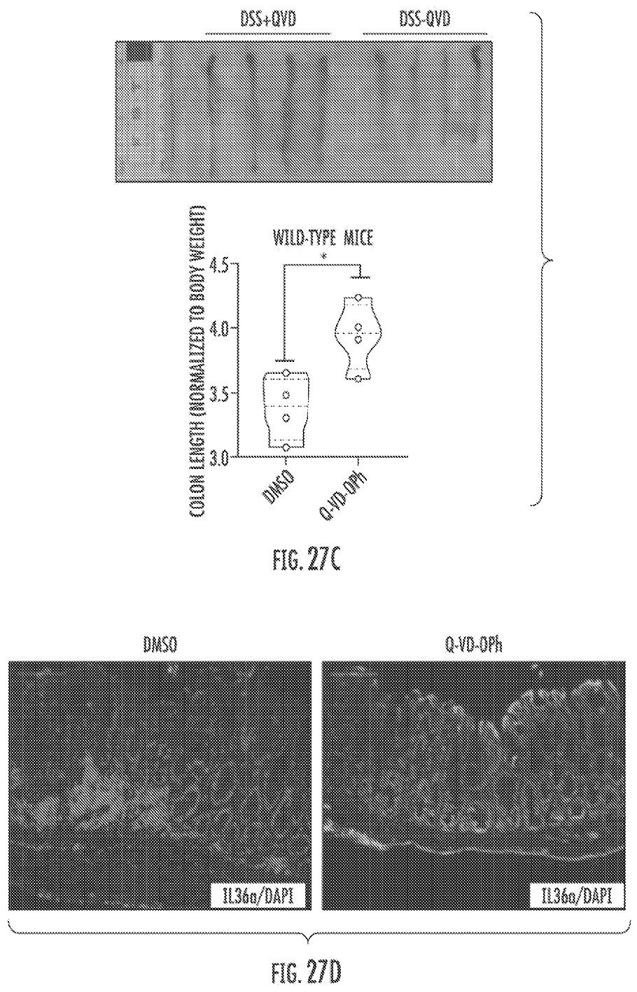

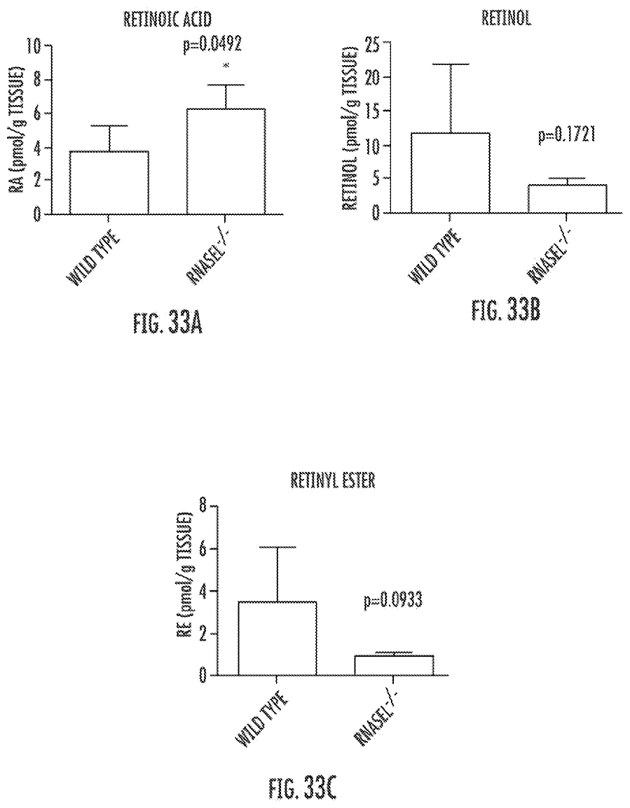

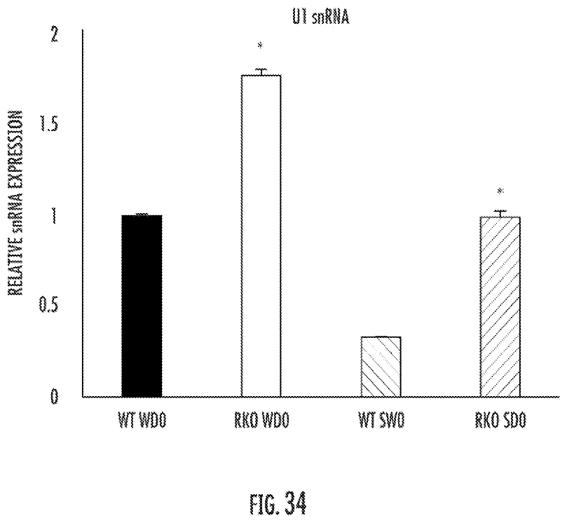

To investigate the role of RNase L in regeneration in vivo, we wounded Rnasel^{-/-} and strain-matched control mice to measure WIHN^{2,8,17}. Consistent with gene expression changes above, Rnasel^{-/-} mice show enhanced regeneration as seen by a greater abundance of de novo hair follicles (FIG. 24A). Despite a higher baseline, Rnasel^{-/-} mice also display an intact regenerative response after addition of exogenous dsRNA. Given the established role of polyI:C to increase WIHN, this additive effect demonstrates a distinct role of RNase L to suppress regeneration (FIG. 24A). Although wound closure speed is not significantly affected by Rnasel loss (FIG. 24B, FIG. 33), Rnasel mice have improved barrier restoration (FIG. 24C), which can partially be explained by RNase L's role as an inhibitor of cell migration¹⁸. Interestingly, Rnasel^{-/-} mice have a thinner hypodermis, which may be explained by the role of RNase L during adipogenesis (FIG. 32)¹⁹. During wound re-epithelialization, Rnasel^{-/-} mice express higher levels of morphogenesis transcripts (Tlr3, 116, Wnt7b, and Edar), consistent with our in vitro findings in human and mouse keratinocytes (FIG. 24D, FIG. 23E, FIG. 26A). Even in unwounded skin, the stem cell markers Krt5 and Krt15 are upregulated in Rnasel^{-/-} mice at baseline, suggesting that these mice are primed to regenerate more robustly (FIG. 24E). Corroborating recent findings that Retinoic Acid (RA) is a morphogen required for regeneration and WIHN⁸, we find elevated levels of RA, but not other retinoid derivatives, in Rnasel^{-/-} mice, as measured by mass spectrometry (FIG. 33A-33C). Also, given recent findings that endogenous non-coding RNAs regulate WIHN, we find elevated levels of the TLR3 agonist, U1 snRNA^{2,8,9,20}, in unwounded and healed wounds of Rnasel^{-/-} mice (FIG. 34). Together, these results demonstrate that RNase L acts as a repressor of regeneration in skin, with loss-of-function resulting in increased numbers of regenerated hair follicles and hair morphogenesis and stem cell markers.

We next focused on defining novel pathways that induce regeneration in Rnasel^{-/-} mice. Gene ontology analysis of Rnasel^{-/-} mice wounds during the re-epithelialization phase of WIHN demonstrate enrichment of neutrophil chemotaxis (FIG. 24F). Indeed, post wounding, Rnasel^{-/-} mice recruit significantly more neutrophils and neutrophil extracellular traps (NETs) to the wound bed than strain-matched controls (FIG. 24F)²¹. Gene ontology analysis also reveals elevated interleukin-1 (Il-1) pathways in Rnasel^{-/-} mice, suggesting that acute inflammation may promote regeneration (FIG. 24F). In sum, this characterization of Rnasel^{-/-} mouse skin suggests that an IL-1 family member capable of increasing neutrophils is responsible for the elevated WIHN.

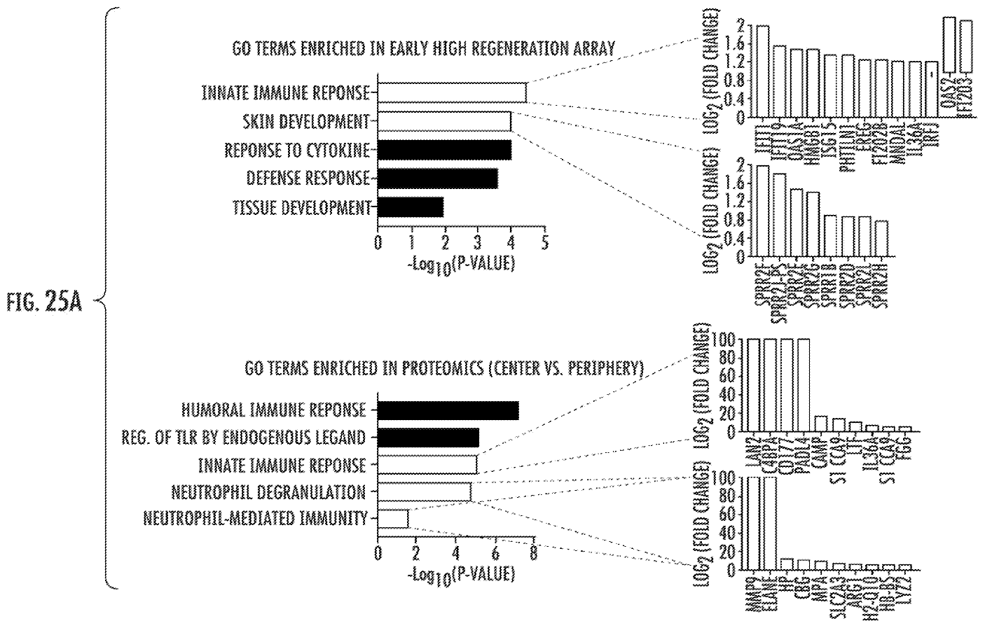

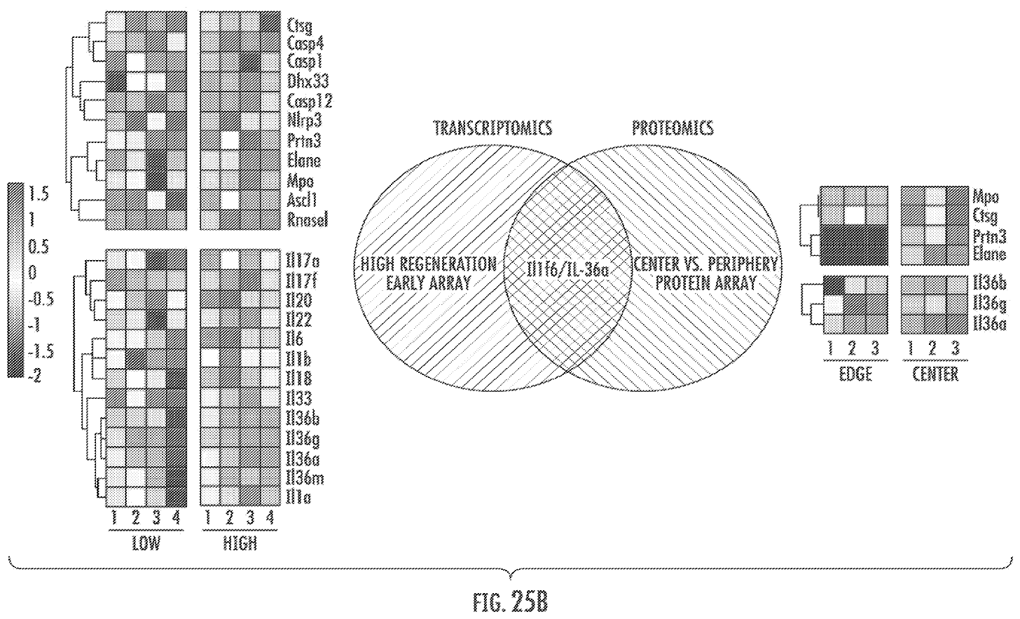

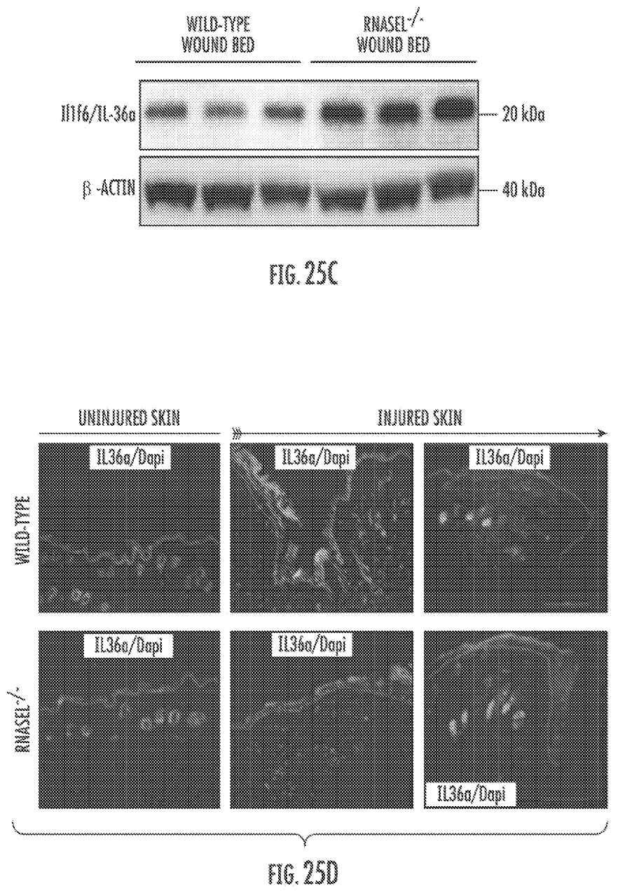

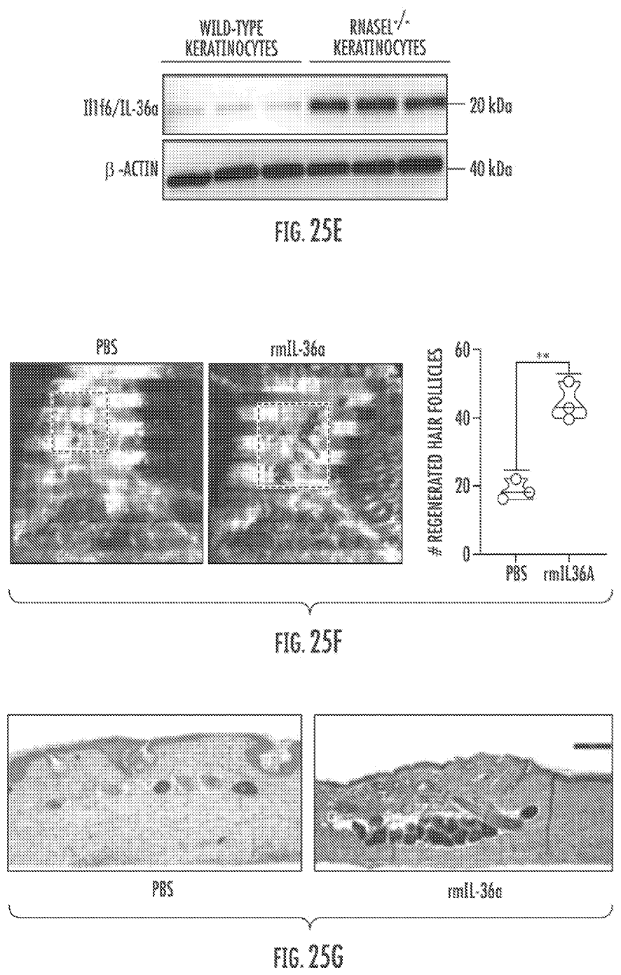

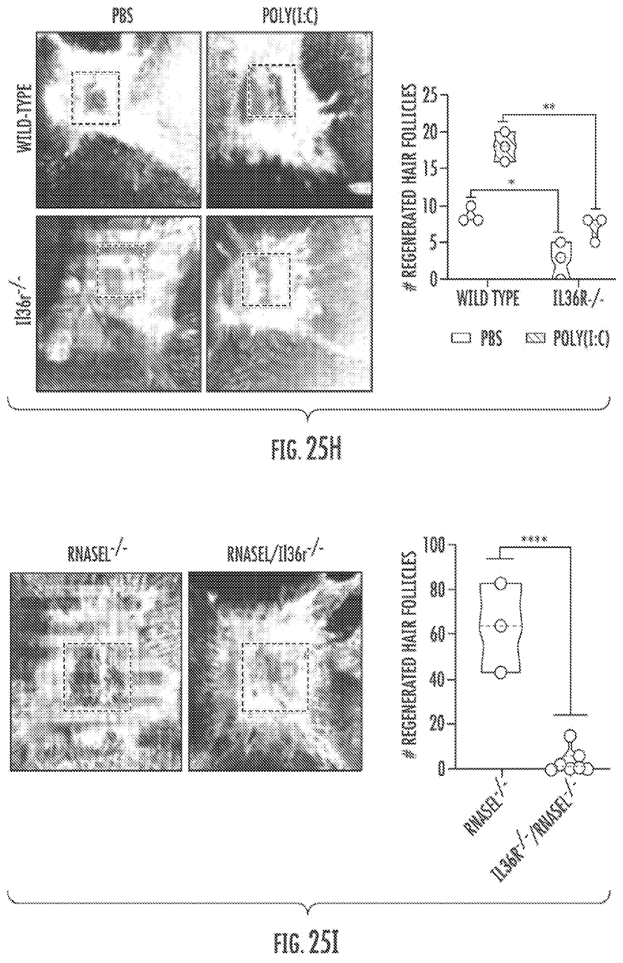

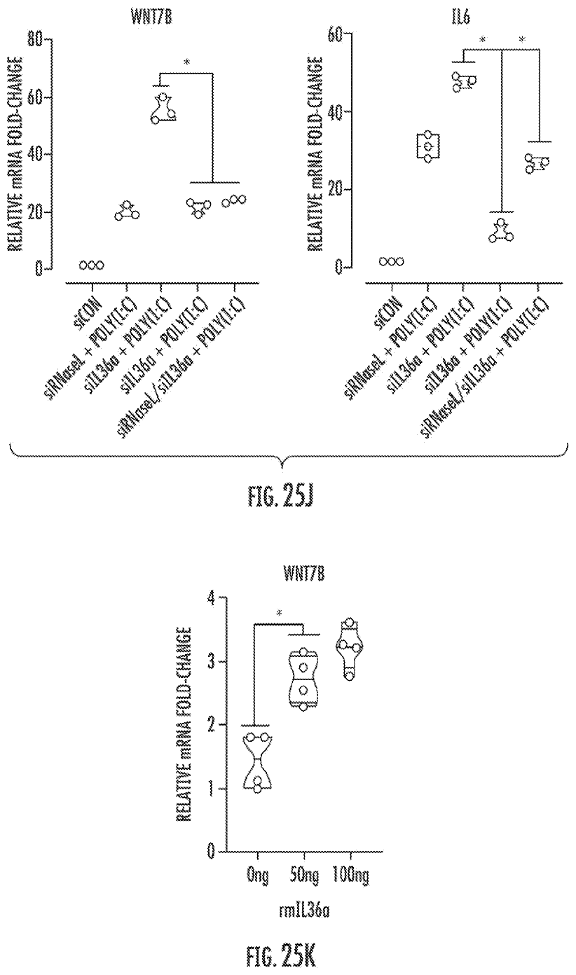

To search for such a candidate that explains the high WIHN in RNaseL KO mice, we next analyzed two scenarios of high WIHN in wild type mice for any that might also be hyperactivated during RNaseL loss of function. In the first, a hallmark of WIHN is the topographical affinity for de novo hair follicles to form in the center rather than the periphery of the wound in mice and rabbits. We determined the top 100 proteins present in the center of the wound as compared to the edge by proteomics⁸. For the second, we determined the top 100 transcripts expressed in healed wounds of outbred mice with high WIHN (C57BL/6×FVB×SJL) compared to a pure C57Bl/6 strain with low WIHN, as mentioned above^{1,5}. In this intersection, we identify the specific Il-1 cytokine family member Il-36 and neutrophil biological processes and enzymes such as elastase (Elane) (FIG. 25A-25B). IL-36α is a DAMP that regulates epidermal inflammation. In humans, genetic defects leading to gain of function of IL-36 are associated with neutrophilic infiltration in the skin²²⁻²⁵. In fact, distinct to other IL-1 family members that are catalytically activated by caspases, the IL-36 family (α, β, γ, and the receptor antagonist, rn) is also activated by neutrophil-derived proteases like cathepsin G (Ctsg) and Elane^{23,26,27}. As would be predicted, Rnasel levels are decreased in the high WIHN outbred mice (FIG. 25B). Therefore, wild type WIHN shows a strong signature for IL-36α and neutrophil enzymes known to activate it (FIG. 25B). This suggested IL-36α as a candidate to explain the high WIHN of Rnasel^{-/-} mice, itself associated with neutrophil infiltration and IL-1 family member GO signature (FIG. 25A).

To test whether IL36 mediates the high WIHN of RNase L null mice, we investigated the role of IL-36 in wild-type, Il36r^{-/-}, and Rnasel^{-/-} mice. After skin injury, we find that Rnasel^{-/-} mice have significantly more IL-36α in the wound bed (FIG. 25C) and that it is highest in the epithelial lip/leading edge of inwardly migrating keratinocytes (FIG. 25D), suggesting its role in healing. This is consistent with findings in vitro where cultured Rnasel^{-/-} keratinocytes secrete elevated levels of total IL-36α (FIG. 25E). Next, we tested the effect of injected recombinant IL-36α (rmIL-36α) protein on WIHN. Compared to vehicle-treated, strain-matched controls, mice treated with rmIL-36α have more than a 2-fold increase in the number of neogenic hair follicles (FIG. 25F-25G). Given the sufficiency of IL-36 to promote WIHN, we tested the functional requirement for IL-36 by wounding mice lacking the receptor for IL-36 (Il36r^{-/-} mice). Il36 receptor loss nearly abolishes regeneration in vehicle-treated, strain-matched controls (FIG. 25H). In fact, the previously observed enhanced WIHN by exogenous dsRNA is also abrogated (FIG. 25H). To define epistasis between RNase L and IL-36, we generated and wounded a double-knockout strain (Rnasel^{-/-}/Il36r^{-/-} mice). Rnasel^{-/-} Il36r^{-/-} mice lose the enhanced regenerative capacity seen in Rnasel^{-/-} mice (FIG. 25I). This suggests that increased IL-36 activity is downstream of RNase L loss. Recapitulating these findings in vitro, we observe simultaneous transcriptional silencing of both RNase L and IL-36α in human keratinocytes results in decreased WNT7B and IL-6 compared to keratinocytes only targeted for RNase L (FIG. 25J). Finally, in human keratinocytes, recombinant IL-36α promotes WNT7B expression (FIG. 25K). Altogether, these results suggest that RNase L loss leads to increased IL-36α production, enhancing WIHN. We next sought to define the mechanism by which IL-36α is induced in response to RNase L deletion.

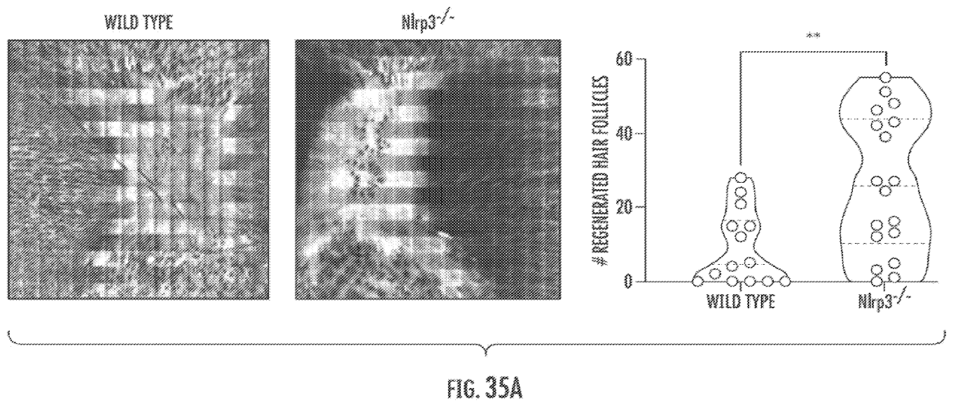

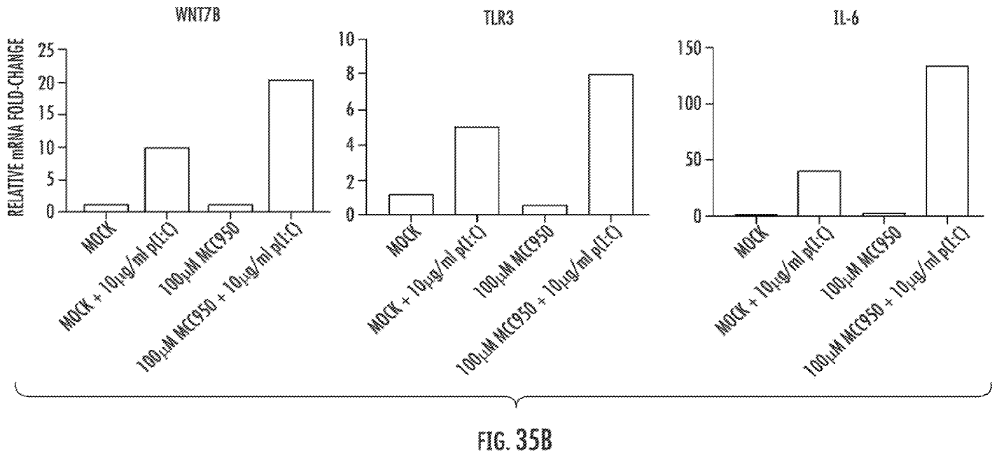

RNase L is a known activator of caspase 1 via the Nlrp3 inflammasome; we thus wondered if IL-36α-mediated WIHN, as a result of RNase L deletion, could instead be directly triggered by caspase inhibition²⁸. First, we tested if Nlrp3 loss would increase regeneration, as occurs with RNase L knockout. Indeed, Nlpr3^{-/-} mice have increased WIHN (FIG. 35A). Similarly, cells treated with the inflammasome inhibitor MCC950 also have greater regeneration markers than controls, which are further increased by exogenous dsRNA addition (FIG. 35B).

We next queried if caspase inhibition functions similarly. Previous reports indicate that there are decreased caspase 1 levels (pro and activated) in stimulated Rnasel null mice^{28,29}. Perhaps as compensation, caspase mRNA is elevated in mouse keratinocytes depleted of Rnasel (FIG. 30C). Although caspases proteolytically process many IL-1 family members such as IL-1β, IL-18, and IL-33, they do not process the IL-36 family^{23,26,27}. Therefore, we hypothesized that caspases may inhibit IL-36α through direct or indirect mechanisms, given the negative RNase L and positive IL-36 correlation with regeneration³⁰. To explore the ability of caspases to basally inhibit IL-36 expression, we performed an siRNA screen of multiple caspases as well as upregulated genes seen when comparing in vivo mouse and in vitro human arrays with Rnasel deletion and depletion, respectively (FIG. 26A-26B). We find that although other caspases and genes may contribute, caspase 1 inhibition consistently results in the highest IL-36α protein levels (FIG. 26B-26C). This suggests that caspase inhibition may be equivalent to RNase L inhibition in WIHN promotion.

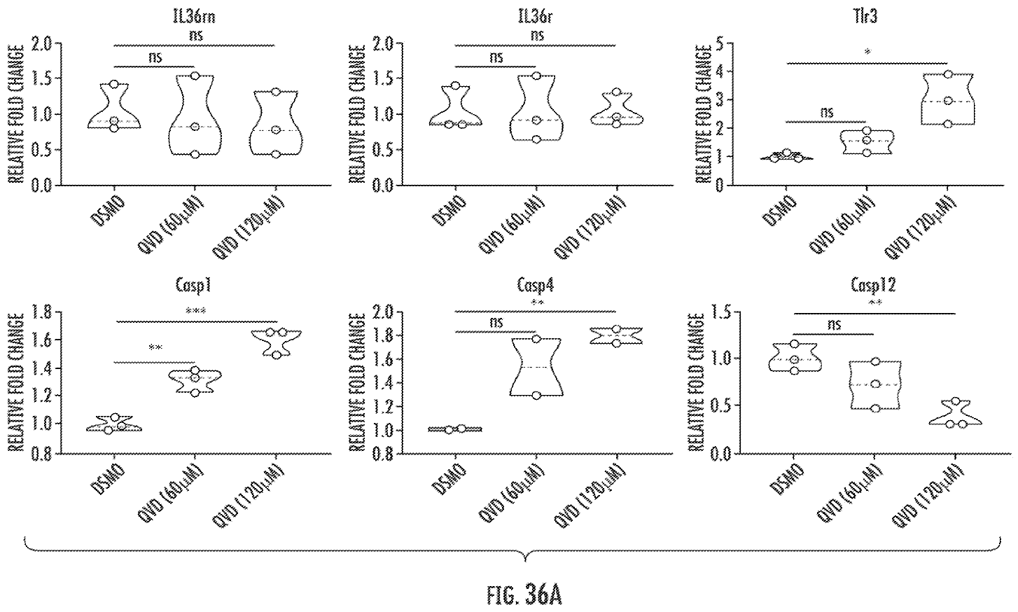

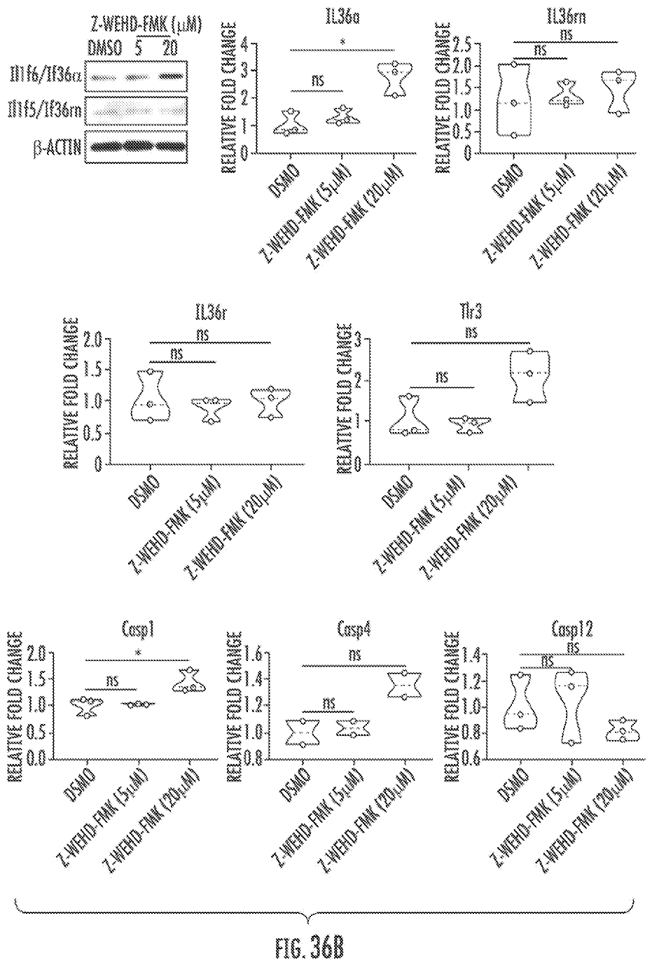

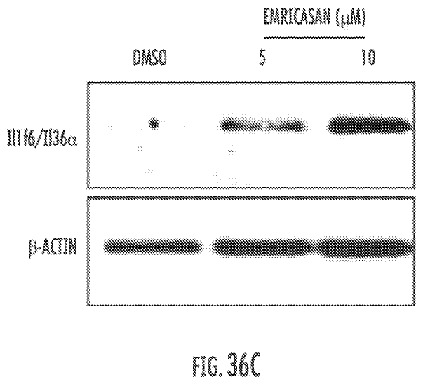

We next tested if small molecule caspase inhibition, which is currently in clinical testing for a number of indications, could promote IL-36 expression, lead to regeneration, and suggest clinical utility. We intraperitoneally injected mice with the irreversible pan-caspase inhibitor Q-VD-OPh prior to wounding (FIG. 26D). Following treatment and FACS analysis, visualization of t-distributed stochastic neighbor embedding (viSNE) clustering reveals higher neutrophil levels in the skin of mice treated with Q-VD-OPh compared to vehicle controls, reminiscent of results from Rnasel^{-/-} mice (FIG. 26E). Mouse epithelial keratinocytes (MEKs) treated with Q-VD-OPh also have higher levels of IL-36α mRNA and protein (FIG. 26F-26G). No differences are seen in the protein or mRNA levels of the IL-36 receptor antagonist (IL-36rn), suggesting that the elevated IL-36α levels upon caspase inhibition are not being mediated via a feedback loop with the antagonist (FIG. 26G and FIG. 36A). Furthermore, differences in the IL-36 receptor protein are not detected. Elevated morphogenesis genes (Tlr3), as seen in Rnasel^{-/-} mice, are again elevated, this time just with caspase inhibition, as are the compensatory mRNA increases of caspase genes (FIG. 36A). These findings were recapitulated with Z-WEHD-FMK, a Group 1 caspase inhibitor that primarily targets caspase 1 (FIG. 36B), as well as the pan-caspase inhibitor Emricasan (FIG. 36C)³¹. In all cases, caspase inhibition yields elevated total IL-36α. Importantly, wild-type mice treated with Q-VD-OPh display more WIHN compared to vehicle-treated mice, in addition to higher levels of IL-36α in the epidermis of re-epithelialized wounds (FIG. 26H-26J), while Il36r^{-/-} mice treated with Q-VD-OPh do not (FIG. 26K-26L). These results collectively demonstrate that caspase inhibition promotes regeneration in an IL-36 and RNase L dependent manner.

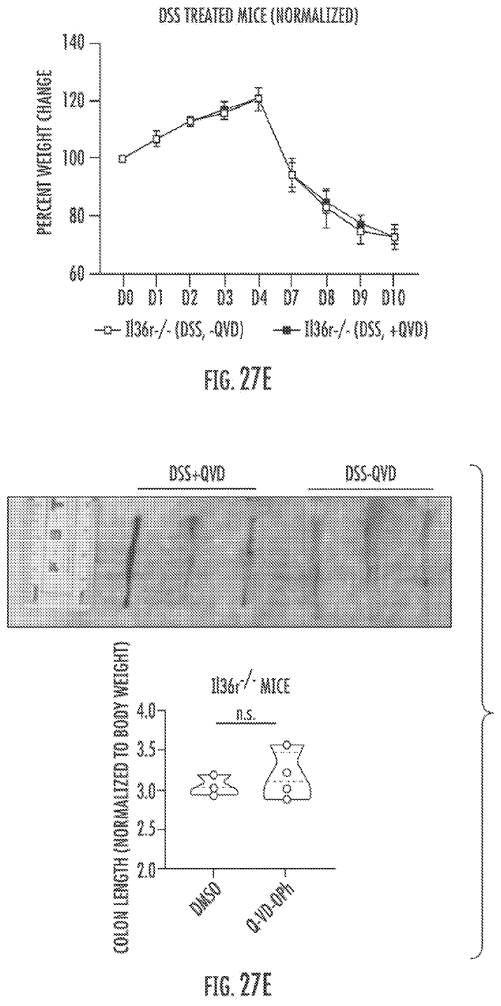

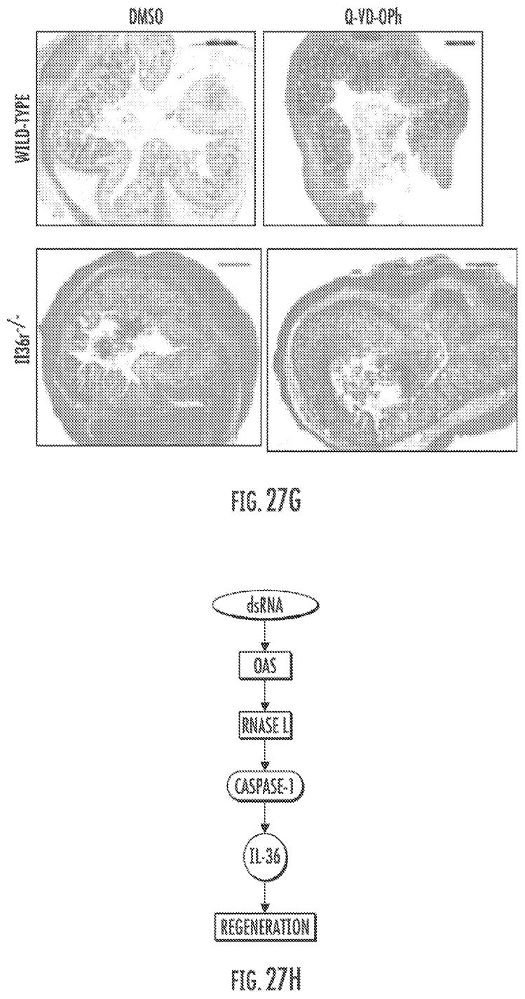

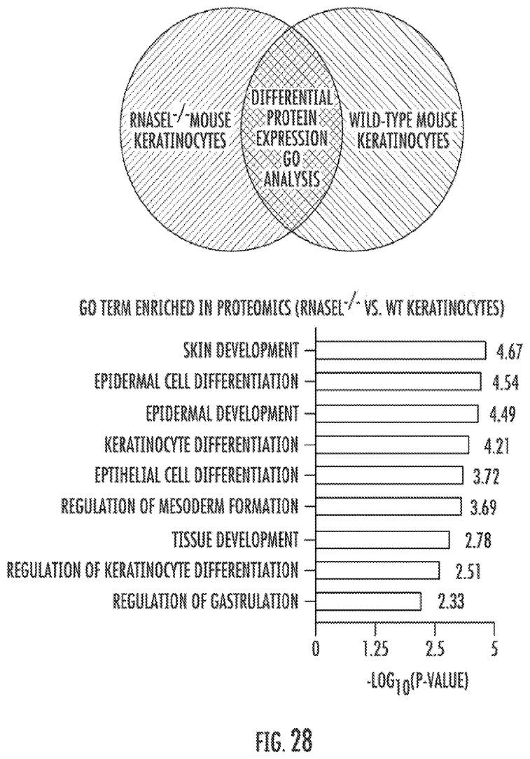

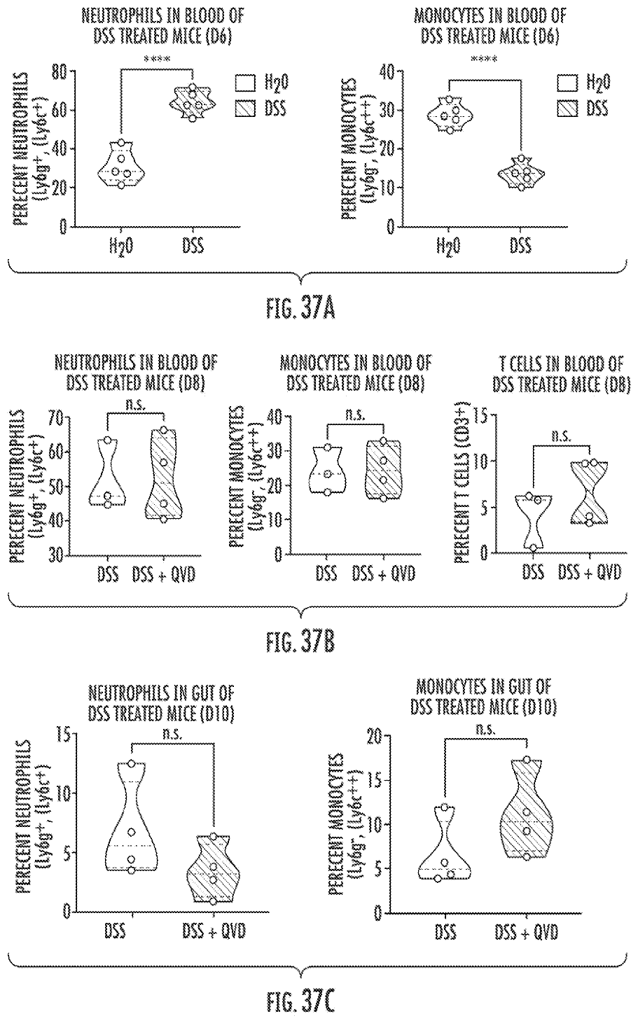

Finally, we sought to determine if the regenerative effects of caspase inhibition are restricted to the skin or could have clinical utility in other organs. We tested if small molecule caspase inhibition could prevent or rescue gut damage in a dextran sulfate sodium (DSS)-induced colitis model (FIG. 27A). Indeed, we find that mice treated with Q-VD-OPh are protected from DSS-induced weight loss and maintain longer and better colon morphology compared to control mice (FIG. 27B-27C, 27G). This effect appears to be the result of enhanced healing and not simply a broad reduction in inflammation caused by pan-caspase inhibition by Q-VD-OPh. While DSS increases circulating neutrophils in WT mice (FIG. 37A), we see no change in neutrophil abundance during the regenerative phase following cessation of DSS between Q-VD-OPh treated mice and controls in the blood or even the gut. (FIG. 37A-37C). Increased IL-36α expression is also observed in the gut of WT mice treated with Q-VD-OPh (FIG. 27D). Similar to our skin observations, however, 1136r^{-/-} mice completely lack the ability to respond to Q-VD-OPh and exhibit similar damage kinetics to control mice (FIG. 27E-27G). Therefore, the inhibition of the RNase L-caspase pathway to promote IL-36 enhances injury response in multiple disparate contexts of organ or tissue injury.

### Discussion

In summary, we have uncovered a novel repressor of regeneration—RNase L—and described the pathway by which caspases inhibit regeneration of multiple mammalian tissues (FIG. 27H). The existence of regeneration repressor genes might partially explain the restricted capacity for adult organogenesis in mammals compared to animals such as axolotl. Q-VD-OPh like compounds may be useful in humans after acute injuries such as skin burns or bowel perforation, that currently have no medical intervention besides general support therapy. Our results are also consistent with the hypothesis that limited regeneration in humans and other mammals is an evolutionary adaptation, possibly to restrict carcinogenesis³². While we show here that loss of RNase L increases regeneration, loss-of function mutations in RNase L have also been implicated as risk factors for cancer³³.

Our data do raise multiple interesting questions for further study. One is defining the exact mechanism of how caspases basally restrain IL36α expression. We demonstrate that this occurs partially through decreased transcription of IL36α, though without changes to IL36 Receptor Antagonist, suggesting that activation of the IL36RN by caspases is less relevant since this would be more downstream. Instead, caspases might basally cleave transcription factors that would otherwise promote IL36α transcription. One such candidate is SP-1, a known substrate for caspase-1, and also predicted to bind a promoter/enhancer for IL-36α (GH02J112875)³⁴. Our data also uncovers an important paradox where Poly I:C promotes WIHN, but the dsRNA activated enzyme RNaseL inhibits WIHN. Therefore, RNaseL independent dsRNA activated pathways are critical for WIHN promotion and might include retinoic acid, IL-6 family members, and likely other important pathways to identify in the future^{2,8}.

Finally, these findings suggest that the inhibition of caspases associated with viral infection and RNaseL activation might have therapeutic benefit to epithelial injury, for example to the lungs of COVID-19 patients. Therefore, pharmacologic targeting of regeneration repressors has many potential novel therapeutic implications.

TABLE 1

| | | | Product |
|---|---|---|---|
| | | | size |
| | | | (base |
| | | | pair: |
| Strain | Primer (5′-3′) | | bp) |
| Rnase1^{-/-} | Forward | GGAGGAGAAGCTTTACAAGGTG | WT: 900 |
| | (HindII) | (SEQ ID NO: 2) | bp |
| | Reverse | GCATTGAGGACCATGGAGAC | KO: 2.1 |
| | (RNase | (SEQ ID NO: 3) | kbp |
| | L) | | |
| I136r^{-/-} | Forward | GCCGCTACACACCACAACCAG | WT: 390 |
| | | (SEQ ID NO: 4) | bp |
| | Reverse | AGTTCAGTAGTCCACTGCCACTC | KO: 520 |
| | | (SEQ ID NO: 5) | bp |

TABLE 2

| Name | Host | Fluorophore | Manufacturer/Product # |
|---|---|---|---|
| CD103 | Rat IgG | BUV805 | BD/741948 |
| CD45 | Rat IgG | BUV563 | BD/565710 |
| CD4 | Rat IgG | BUV496 | BD/564667 |
| GR1+ | Rat IgG | BUV395 | BD/566218 |
| CD11b | Rat IgG | BV786 | BD/740861 |
| MHC-II | Rat IgG | BV711 | BD/563414 |
| F4/80 | Rat IgG | BV650 | BD/743282 |
| gdTCR | Hamster IgG | BV605 | BD/744116 |
| Live/Dead | | Zombie-Aqua | BioLegend/423101 |
| Ly6G | Rat IgG | BV421 | BD/562727 |
| CD11c | Rat IgG | PerCP-Vio700 | Miltenyi/130-103-806 |
| Ly6G | Rat IgG | PE-Cy7 | BD/560601 |
| CD3 | Hamster IgG | PE-CF594 | BD/562332 |
| CD163 | Rat IgG | PE | Thermo Fisher/12-1631-82 |
| CD115 | Rat IgG | APC-Cy7 | BioLegend/135532 |
| CD8 | Rat IgG | AF700 | BD/557959 |
| CD207 | Rat IgG | eFluor660 | eBioscience/50-2073-82 |

TABLE 3

| | | Dilution | | |
|---|---|---|---|---|
| | | Western | Immuno- | Company/ |
| Name | Host | blot | staining | Product # |
| WNT7B | Rabbit | | 1:200 | Thermo Fisher/ |
| | | | | PA5-30207 |
| KRT5 | Mouse | | | Abcam/ab17130 |
| KRT15 | Rabbit | | 1:300 | Sigma/HPA023910 |
| IL-36α | Rabbit | | 1:100 | Abcam/ab180909 |
| IL-36α | Goat | 1:1000 | | R&D Systems/ |
| | | | | AF1078 |
| IL-36γ | Goat | 1:1000 | | R&D Systems/ |
| | | | | AF2320 |
| β-actin | Rabbit | 1:1000 | | Cell Signaling |
| | | | | Technology/4967L |
| Histone H4 | Rabbit | | 1:500 | Millipore Sigma/ |
| (citrulline 3) | | | | 07-596 |
| Alexa | Rabbit | | 1:1000 | Invitrogen/ |
| Fluor ® 488 | | | | A27012 |
| Anti-Goat | | | | |
| IgG (H + L) | | | | |
| Alexa | Goat | | 1:1000 | Invitrogen/ |
| Fluor ® 488 | | | | A-11008 |
| Anti-Rabbit | | | | |
| IgG (H + L) | | | | |
| Alexa | Goat | | 1:1000 | Invitrogen/ |
| Fluor ® 594 | | | | A-11037 |
| Anti-Rabbit | | | | |
| IgG (H + L) | | | | |
| anti-mouse | Horse | 1:1000 | | Cell Signaling |
| IgG-HRP | | | | Technology/7076S |
| anti-rabbit | Goat | 1:1000 | | Cell Signaling |
| IgG-HRP | | | | Technology/7074S |

TABLE 4

| | Host | | |
|---|---|---|---|
| Gene Name | Target | Type | Manufacturer/Product # |
| RNase L | Human | SMARTpool | Dharmacon/M-005032-01-0005 |
| | | siGENOME | |
| IL36A | Human | SMARTpool | Dharmacon/M-007956-03-0005 |
| | | siGENOME | |
| Caspase 1 | Mouse | SMARTpool | Dharmacon/M-048913-01-0005 |
| | | siGENOME | |
| Caspase 2 | Mouse | SMARTpool | Dharmacon/M-044184-00-0005 |
| | | siGENOME | |
| Caspase 3 | Mouse | SMARTpool | Dharmacon/M-043042-01-0005 |
| | | siGENOME | |
| Caspase 4 | Mouse | SMARTpool | Dharmacon/M-042432-01-0005 |
| | | siGENOME | |
| Caspase 6 | Mouse | SMARTpool | Dharmacon/M-063186-01-0005 |
| | | siGENOME | |
| Caspase 7 | Mouse | SMARTpool | Dharmacon/M-057362-01-0005 |
| | | siGENOME | |
| Caspase 8 | Mouse | SMARTpool | Dharmacon/M-043044-01-0005 |
| | | siGENOME | |
| Caspase 9 | Mouse | SMARTpool | Dharmacon/M-040674-01-0005 |
| | | siGENOME | |
| Caspase 12 | Mouse | SMARTpool | Dharmacon/M-062480-01-0005 |
| | | siGENOME | |
| Caspase 14 | Mouse | SMARTpool | Dharmacon/M-062483-01-0005 |
| | | siGENOME | |
| Non- | Human/ | SMARTpool | Dharmacon/D-001206-13-05 |
| targeting | Mouse | siGENOME | |
| Control | | | |

### REFERENCES

1. Ito, M. et al., Wnt-dependent de novo hair follicle regeneration in adult mouse skin after wounding. Nature 447, 316-320 (2007).
2. Nelson, A. M. et al., dsRNA Released by Tissue Damage Activates TLR3 to Drive Skin Regeneration. Cell stem cell 17, 139-151 (2015).
3. Brockes, J. P. Amphibian limb regeneration: rebuilding a complex structure. Science 276, 81-87 (1997).
4. Tanaka, H. V. et al., A developmentally regulated switch from stem cells to dedifferentiation for limb muscle regeneration in newts. Nature communications 7, 11069 (2016).
5. Breedis, C. Regeneration of hair follicles and sebaceous glands from the epithelium of scars in the rabbit. Cancer research 14, 575-579 (1954).
6. Lim, C. H. et al., Hedgehog stimulates hair follicle neogenesis by creating inductive dermis during murine skin wound healing. Nature communications 9, 4903 (2018).
7. Gay, D. et al., Fgf9 from dermal gammadelta T cells induces hair follicle neogenesis after wounding. Nature medicine 19, 916-923 (2013).
8. Kim, D. et al., Noncoding dsRNA induces retinoic acid synthesis to stimulate hair follicle regeneration via TLR3. Nature communications 10, 2811 (2019).
9. Bernard, J. J. et al., Ultraviolet radiation damages self noncoding RNA and is detected by TLR3. Nature medicine 18, 1286-1290 (2012).
10. Silverman, R. H. Viral encounters with 2′,5′-oligoadenylate synthetase and RNase L during the interferon antiviral response. J Virol 81, 12720-12729 (2007).
11. Banerjee, S., Chakrabarti, A., Jha, B. K., Weiss, S. R. & Silverman, R. H. Cell-type-specific effects of RNase L on viral induction of beta interferon. mBio 5, e00856-00814
12. Zhou, A., Hassel, B. A. & Silverman, R. H. Expression cloning of 2-5A-dependent RNAase: a uniquely regulated mediator of interferon action. Cell 72, 753-765 (1993).
13. Liu, C. X. et al., Structure and Degradation of Circular RNAs Regulate PKR Activation in Innate Immunity. Cell 177, 865-880 e821 (2019).
14. Malathi, K., Dong, B., Gale, M., Jr. & Silverman, R. H. Small self-RNA generated by RNase L amplifies antiviral innate immunity. Nature 448, 816-819 (2007).
15. Yang, Z. G. et al., The Circular RNA Interacts with STAT3, Increasing Its Nuclear Translocation and Wound Repair by Modulating Dnmt3a and miR-17 Function. Molecular therapy: the journal of the American Society of Gene Therapy 25, 2062-2074 (2017).
16. Sledz, C. A., Holko, M., de Veer, M. J., Silverman, R. H. & Williams, B. R. Activation of the interferon system by short-interfering RNAs. Nat Cell Biol 5, 834-839 (2003).
17. Zhu, A. S., Li, A., Ratliff, T. S., Melsom, M. & Garza, L. A. After Skin Wounding, Noncoding dsRNA Coordinates Prostaglandins and Wnts to Promote Regeneration. The Journal of investigative dermatology 137, 1562-1568 (2017).
18. Banerjee, S. et al., RNase L is a negative regulator of cell migration. Oncotarget 6, 44360-44372 (2015).
19. Wang, Y. T. et al., A link between adipogenesis and innate immunity: RNase-L promotes 3T3-L1 adipogenesis by destabilizing Pref-1 mRNA. Cell death & disease 7, e2458 (2016).
20. Borkowski, A. W. et al., Toll-like receptor 3 activation is required for normal skin barrier repair following UV damage. The Journal of investigative dermatology 135, 569-578 (2015).
21. Brinkmann, V. et al., Neutrophil extracellular traps kill bacteria. Science 303, 1532-1535 (2004).
22. Bassoy, E. Y., Towne, J. E. & Gabay, C. Regulation and function of interleukin-36 cytokines. Immunological reviews 281, 169-178 (2018).
23. Henry, C. M. et al., Neutrophil-Derived Proteases Escalate Inflammation through Activation of IL-36 Family Cytokines. Cell reports 14, 708-722 (2016).
24. Mahil, S. K. et al., An analysis of IL-36 signature genes and individuals with IL1RL2 knockout mutations validates IL-36 as a psoriasis therapeutic target. Science translational medicine 9(2017).
25. Mantovani, A., Dinarello, C. A., Molgora, M. & Garlanda, C. Interleukin-1 and Related Cytokines in the Regulation of Inflammation and Immunity. Immunity 50, 778-795 (2019).
26. Macleod, T. et al., Neutrophil Elastase-mediated proteolysis activates the anti-inflammatory cytokine IL-36 Receptor antagonist. Sci Rep 6, 24880 (2016).
27. Towne, J. E. et al., Interleukin-36 (IL-36) ligands require processing for full agonist (IL-36alpha, IL-36beta, and IL-36gamma) or antagonist (IL-36Ra) activity. J Biol Chem 286, 42594-42602 (2011).
28. Chakrabarti, A. et al., RNase L activates the NLRP3 inflammasome during viral infections. Cell host & microbe 17, 466-477 (2015).
29. Rusch, L., Zhou, A. & Silverman, R. H. Caspase-dependent apoptosis by 2′,5′-oligoadenylate activation of RNase L is enhanced by IFN-beta. J Interferon Cytokine Res 20, 1091-1100 (2000).
30. Afonina, I. S., Muller, C., Martin, S. J. & Beyaert, R. Proteolytic Processing of Interleukin-1 Family Cytokines: Variations on a Common Theme. Immunity 42, 991-1004 (2015).
31. Garcia-Calvo, M. et al., Inhibition of human caspases by peptide-based and macromolecular inhibitors. J Biol Chem 273, 32608-32613 (1998).
32. Liu, W., Liang, S. L., Liu, H., Silverman, R. & Zhou, A. Tumour suppressor function of RNase L in a mouse model. European journal of cancer 43, 202-209 (2007).
33. Carpten, J. et al., Germline mutations in the ribonuclease L gene in families showing linkage with HPC1. Nature genetics 30, 181-184 (2002).
34. Torabi, B. et al., Caspase cleavage of transcription factor Spl enhances apoptosis. Apoptosis 23, 65-78 (2018).
35. Zhou, A. et al., Interferon action and apoptosis are defective in mice devoid of 2′,5′-oligoadenylate-dependent RNase L. The EMBO journal 16, 6355-6363 (1997).
36. Kim, S. et al., Simple cell culture media expansion of primary mouse keratinocytes. Journal of dermatological science 93, 135-138 (2019).
37. Wirtz, S. et al., Chemically induced mouse models of acute and chronic intestinal inflammation. Nature protocols 12, 1295-1309 (2017).
38. Fong, J. H., Murphy, T. D. & Pruitt, K. D. Comparison of RefSeq protein-coding regions in human and vertebrate genomes. BMC Genomics 14, 654 (2013).
39. Ritchie, M. E. et al., limma powers differential expression analyses for RNA-sequencing and microarray studies. Nucleic Acids Res 43, e47 (2015).
40. Yu, G., Wang, L. G., Han, Y. & He, Q. Y. clusterProfiler: an R package for comparing biological themes among gene clusters. OMICS 16, 284-287 (2012).

That which is claimed:

1. A method for treating a bacterial infection in a patient comprises the step of administering to the patient an effective amount of a caspase inhibitor.

2. A method for treating a bacterial skin lesion in a patient comprises the step of administering to the patient an effective amount of a caspase inhibitor.

3. A method for enhancing regeneration and repair after skin or gut injury in a patient comprises the step of administering an effective amount of a caspase inhibitor.

4. A method for treating a viral infection in a patient comprises the step of administering to the patient an effective amount of a caspase inhibitor.

5. The method of claim 4, wherein the viral infection is COVID19.

6. The method of any of claims 1-5, wherein the caspase inhibitor is a pan caspase inhibitor.

7. The method of any of claims 1-6, wherein the caspase inhibitor is Q-VD-OPh.

8. The method of claim 3, wherein the skin or gut injury is a scar or a burn.

9. The method of claim 3, further comprising the step of administering a TLR3 agonist.

10. The use of a caspase inhibitor for the preparation of a medicament for the treatment of a bacterial infection.

11. The use of a caspase inhibitor for the preparation of a medicament for the treatment of a bacterial skin lesion.

12. The use of a caspase inhibitor for the preparation of a medicament for the treatment of skin or gut injury.

13. The use of a caspase inhibitor for the preparation of a medicament for the treatment of a scar or a burn.

14. The use of a caspase inhibitor for the preparation of a medicament for the treatment of a viral infection.

15. The use of a caspase inhibitor for the preparation of a medicament for the treatment of COVID-19.
